(19) United States
(12) Patent Application Publication
(10) Pub. No.: US 2022/0192774 A1
(43) Pub. Date: Jun. 23, 2022

(54) KNEE INSTRUMENTS AND METHODS

(71) Applicant: E. Marlowe Goble, (US)

(72) Inventors: E. Marlowe GOBLE, Logan, UT (US); Daniel J. TRIPLETT, Smithfield, UT (US)

(21) Appl. No.: 17/691,034

(22) Filed: Mar. 9, 2022

Related U.S. Application Data
(63) Continuation-in-part of application No. 17/023,988, filed on Sep. 17, 2020.
Application No. 17/023,988 is a continuation-in-part of application No. 16/287,976, filed on Feb. 27, 2019, now Pat. No. 11,234,720.
(60) Provisional application No. 63/158,839, filed on Mar. 9, 2021.
(60) Provisional application No. 63/195,022, filed on May 30, 2021.
(60) Provisional application No. 62/987,153, filed on Mar. 9, 2020.
(60) Provisional application No. 62/902,892, filed on Sep. 19, 2019.
(60) Provisional application No. 62/640,006, filed on Mar. 7, 2018.

(51) Int. Cl. A61B 90/00; A61B 17/15; A61B 17/17; A61F 2/38; A61B 34/10

(52) U.S. Cl. CPC A61B 90/06; A61B 17/154; A61B 17/1764; A61B 2034/108; A61F 2/3859; A61B 34/10; A61B 2090/067; A61F 2/389

(57) ABSTRACT

An angle gauge may be used to facilitate arthroplasty of a knee joint between a femur and a tibia. The angle gauge may have a base configured to contact a distal end of the femur, a post pivotably connected to the base such that the post is visually alignable with Whiteside's Line on the femur, and an angle capture feature configured to capture an angle of Whiteside's Line when the post is in alignment with Whiteside's Line. The post may be part of a sizing guide that indicates an appropriate size of a femoral arthroplasty implant. According to one method for carrying out knee arthroplasty, a distal femur may first be exposed. The orientation of Whiteside's Line on the distal femur may be assessed. Based on the orientation, a position and/or orientation may be defined for one or more resections to be made to the distal femur.

### CROSS-REFERENCE TO RELATED APPLICATIONS

The present application claims the benefit of: U.S. Provisional Application No. 63/158,839, filed Mar. 9, 2021, entitled LINKED INSTRUMENTATION FOR TOTAL KNEE ARTHROPLASTY, and U.S. Provisional Application No. 63/195,022, filed May 30, 2021, entitled LINKED INSTRUMENTATION FOR TOTAL KNEE ARTHROPLASTY.

The present application is also a continuation-in-part of:

U.S. patent application Ser. No. 17/023,988, filed Sep. 17, 2020, entitled KNEE INSTRUMENTS AND METHODS, which claims the benefit of U.S. Provisional Application No. 62/902,892, filed Sep. 19, 2019, entitled KNEE INSTRUMENTS AND METHODS, and U.S. Provisional Application No. 62/987,153, filed Mar. 9, 2020, entitled KNEE INSTRUMENTS AND METHODS.

The present application is a continuation-in-part of:

U.S. patent application Ser. No. 16/287,976, filed Feb. 27, 2019, entitled KNEE INSTRUMENTS AND METHODS, which claims the benefit of U.S. Provisional Application No. 62/640,006, filed Mar. 7, 2018, entitled KNEE INSTRUMENTS AND METHODS.

The present application is related to:

U.S. patent application Ser. No. 15/630,555, filed Jun. 22, 2017, entitled KNEE INSTRUMENTS AND METHODS, which issued as U.S. Pat. No. 10,568,650 on Feb. 25, 2020, which claims the benefit of U.S. Provisional Application No. 62/353,553, filed Jun. 22, 2016, entitled KNEE INSTRUMENTS AND METHODS. U.S. patent application Ser. No. 15/630,555 is a continuation-in-part of U.S. patent application Ser. No. 15/081,828, filed Mar. 25, 2016, entitled KNEE INSTRUMENTS AND METHODS, which issued as U.S. Pat. No. 10,426,565 on Oct. 1, 2019, which claims the benefit of U.S. Provisional Patent Application No. 62/138,307, filed Mar. 25, 2015, entitled KNEE INSTRUMENTS AND METHODS, and

U.S. Provisional Patent Application No. 62/302,787, filed Mar. 2, 2016, entitled KNEE INSTRUMENTS AND METHODS.

The foregoing are incorporated by reference as though set forth herein in their entirety.

### TECHNICAL FIELD

The present disclosure relates to instruments and methods to improve femoral and tibial alignment during knee arthroplasty. More specifically, the present disclosure relates to multi-pin guides and multi-cut guides.

### BACKGROUND

The success of knee arthroplasty is largely dependent upon the accuracy of the distal femoral and proximal tibial bone cuts or resections. When the resections are well aligned to the mechanical axis of the leg and patient knee geometry and orientation, the reconstructed knee functions better and lasts longer. The problem to be solved is to devise instruments and methods which provide exceptional accuracy in combination with simplicity, speed, and value.

### SUMMARY

The various systems and methods of the present technology have been developed in response to the present state of the art, and in particular, in response to the problems and needs in the art that have not yet been fully solved by currently available knee arthroplasty instrument systems and methods. The systems and methods of the present technology may provide improved anatomical referencing, sizing, and alignment comparable to computerized navigation systems, at a fraction of the cost.

To achieve the foregoing, and in accordance with the technology as embodied and broadly described herein, an angle gauge may be configured to facilitate arthroplasty of a knee joint between a femur and a tibia. The angle gauge may have a base configured to contact a distal end of the femur, a post pivotably connected to the base such that the post is visually alignable with Whiteside's Line on the femur, and an angle capture feature configured to capture an angle of Whiteside's Line when the post is in alignment with Whiteside's Line.

The base may be configured to contact one or both posterior condylar surfaces of the femur.

The angle gauge may further include an actuator that can be actuated to change an orientation of the post relative to the base.

The angle capture feature may include a scale on which the angle is indicated.

The post may have one or more holes that are alignable with Whiteside's Line.

The angle gauge may further have one or more pins that can be inserted through the one or more holes in the post to directly reference Whiteside's Line to facilitate alignment of the post with Whiteside's Line.

The post may be incorporated into a sizing guide configured to indicate an appropriate size of a femoral implant to be used in arthroplasty of the knee joint.

The sizing guide may include a size indicator carried by the post at an adjustable displacement from the base to indicate a size of a femoral implant that is suitable for the arthroplasty.

The post may have an anterior plunger that is slidable, within a body, such that the size indicator is alignable with any of a plurality of markings on the body, indicating potential sizes of the femoral implant.

At least one of the post and the body may have a slot through which Whiteside's Line is viewable.

According to some embodiments, a sizing guide may be configured to facilitate arthroplasty of a knee joint between a femur and a tibia. The sizing guide may have a base configured to contact the femur, a post pivotably connected to the base such that the post is visually alignable parallel to Whiteside's Line on the femur, and a size indicator carried by the post at an adjustable displacement from the base to indicate a size of a femoral implant that is suitable for the arthroplasty.

The sizing guide may further have a body. The post may be an anterior plunger that is slidable, within the body, such that the size indicator is alignable with any of a plurality of markings on the body, indicating potential sizes of the femoral implant.

At least one of the post and the body may have a slot through which Whiteside's Line is viewable.

The base may be configured to register on posterior condylar surfaces of the femur.

The sizing guide may further have an anterior arm on an anterior end of the post. The anterior arm may extend from the post to register on an anterior surface of a distal end of the femur.

According to some embodiments, a method for carrying out knee arthroplasty may include exposing a distal femur, assessing an orientation of Whiteside's Line on the distal femur, and, based on the orientation, defining a position and/or orientation of one or more resections to be made to the distal femur.

Assessing the orientation of Whiteside's Line may include visually aligning a post of an angle gauge with Whiteside's Line.

The angle gauge may further include a base to which the post is pivotably coupled. Visually aligning the post with Whiteside's Line may include pivoting the post relative to the base until the post is parallel to Whiteside's Line.

The angle gauge may further include an angle capture feature. The method may further include using the angle capture feature to capture the orientation.

The method may further include receiving the orientation in a surgical robot, and using the orientation to identify a position and/or orientation of one or more resections to be made to the distal femur.

These and other features and advantages of the present technology will become more fully apparent from the following description and appended claims, or may be learned by the practice of the technology as set forth hereinafter.

### BRIEF DESCRIPTION OF THE DRAWINGS

Exemplary embodiments of the technology will become more fully apparent from the following description and appended claims, taken in conjunction with the accompanying drawings. Understanding that these drawings depict only exemplary embodiments and are, therefore, not to be considered limiting of the scope of the technology, the exemplary embodiments will be described with additional specificity and detail through use of the accompanying drawings in which:

FIG. 1 is an oblique view of a multi-pin guide assembly;

FIG. 2 is another oblique view of the multi-pin guide assembly of FIG. 1 from a different direction;

FIG. 3 is an oblique exploded view of the multi-pin guide assembly of FIG. 1;

FIG. 4 is another oblique exploded view of the multi-pin guide assembly of FIG. 1 from a different direction;

FIG. 5 is an oblique view of a femoral multi-pin guide of the multi-pin guide assembly of FIG. 1;

FIG. 6 is another oblique view of the femoral multi-pin guide of FIG. 5 from a different direction;

FIG. 7 is an oblique view of a tibial pin guide of the multi-pin guide assembly of FIG. 1;

FIG. 8 is another oblique view of the tibial pin guide of FIG. 7 from a different direction;

FIG. 9 is an oblique view of the multi-pin guide assembly of FIG. 1 coupled to a femur and tibia by pins, and coupled to a body, a base, a handle, a femoral extension rod, a femoral target assembly, a femoral support arm assembly, and a foot holder assembly, with selected portions of soft tissues shown for context;

FIG. 10 is a lateral view of the multi-pin guide assembly, femur, tibia, pins, body, base, handle, femoral extension rod, femoral target assembly, femoral support arm assembly, and foot holder assembly of FIG. 9, soft tissues omitted for clarity;

FIG. 11A is an anterior or top view of the multi-pin guide assembly, femur, tibia, pins, body, base, handle, femoral extension rod, femoral target assembly, femoral support arm assembly, and foot holder assembly of FIG. 10; FIG. 11B is an enlarged detail view of the of the femur, body, base, handle, femoral extension rod, femoral target assembly, and femoral support arm assembly of FIG. 11A in the vicinity of the femoral head; FIG. 11C is an enlarged detail view of the multi-pin guide assembly, femur, tibia, pins, body, base, handle, and foot holder assembly of FIG. 11A in the vicinity of the knee joint; and FIG. 11D is an enlarged detail view of the multi-pin guide assembly, tibia, and foot holder assembly of FIG. 11A in the vicinity of the ankle joint;

FIG. 12 is an oblique detail view of the multi-pin guide assembly, femur, tibia, pins, body, base, and handle of FIG. 10 from an anterior-inferior-medial direction;

FIG. 13 is an oblique detail view of the femur, tibia, and pins of FIG. 12 after removing the multi-pin guide assembly, body, base, and handle;

FIG. 14 is an oblique detail view of the femur, tibia, and selected pins of FIG. 13, with a distal femoral cut guide coupled to anterior distal femoral pins;

FIG. 15A is an oblique detail view of the femur, tibia, and selected pins of FIG. 13, after making a distal femoral resection and removing the distal femoral cut guide and anterior distal femoral pins, and after attaching a femoral four-in-one cut guide and a tibial cut guide coupled to a tibial extension rod, coupled to the foot holder assembly of FIG. 9; and FIG. 15B is an enlarged oblique detail view of the femur, tibia, femoral four-in-one cut guide, tibial pin, tibial cut guide, and tibial extension rod of FIG. 15A;

FIG. 16 is an oblique view of a femur, a tibia, a fibula, and foot bones, the tibia, fibula, and foot bones secured within a foot holder, a femoral head finder positioned over a center of a head of the femur and coupled to a support;

FIG. 17 is an oblique view of the femur and support of FIG. 16, a femoral pin guide positioned against a central distal anterior cortical portion of the femur, a femoral extension rod coupled to the femoral pin guide and received in a target positioned over the center of the femoral head, the target coupled to the support, and bone pins inserted through the femoral pin guide into a distal aspect of the femur;

FIG. 18 is an enlarged view of a distal portion of the femur, the femoral pin guide, a distal portion of the femoral extension rod, and bone pins of FIG. 17;

FIG. 19 is an anterior view of the femur, a portion of the support, femoral pin guide, femoral extension rod, target, and bone pins of FIG. 17;

FIG. 20 is a lateral view of a portion of the support, femoral pin guide, femoral extension rod, target, and bone pins of FIG. 17;

FIG. 21 is an oblique view of the femur, tibia, fibula, foot bones, foot holder of FIG. 16 with the bone pins of FIG. 17, with a 6-in-1 pin guide coupled to the bone pins, abutting a distal aspect of the femur, and contacting a proximal aspect of the tibia, with a tibial alignment rod coupled to the 6-in-1 pin guide and received in a target positioned over a center of the ankle joint, or over another anatomical landmark of the tibia, ankle, or foot;

FIG. 22 is an enlarged view of a distal portion of the femur, a proximal portion of the tibia, bone pins, 6-in-1 pin guide, and a proximal portion of the tibial alignment rod of FIG. 21;

FIG. 23 is an oblique view of the bone pins and 6-in-1 pin guide of FIG. 22;

FIG. 24 is another oblique view of the bone pins and 6-in-1 pin guide of FIG. 22 from a different direction;

FIG. 25 is an oblique exploded view of a tibial extension rod of the 6-in-1 pin guide of FIG. 23;

FIG. 26 is another oblique exploded view of the tibial extension rod of FIG. 25 from a different direction;

FIG. 27 is an oblique exploded view of the bone pins and a femoral portion of the 6-in-1 pin guide of FIG. 23;

FIG. 28 is another oblique exploded view of the femoral portion of FIG. 27, from a different direction;

FIG. 29 is an oblique exploded view of a tibial portion of the 6-in-1 pin guide of FIG. 23;

FIG. 30 is another oblique exploded view of the tibial portion of FIG. 29, from a different direction;

FIG. 31 is an oblique view of the femur, tibia, bone pins, and 6-in-1 pin guide of FIG. 22 with two bone pins inserted through the 6-in-1 pin guide into a distal anterior aspect of the femur and two bone pins inserted through the 6-in-1 pin guide into a proximal anterior aspect of the tibia, for a total of 6 pins inserted into the femur and tibia;

FIG. 32 is an oblique view of the femur, tibia, and 6 bone pins of FIG. 31 after removing the 6-in-1 pin guide;

FIG. 33 is an oblique view of a cut guide;

FIG. 34 is another oblique view of the cut guide of FIG. 33 from a different direction;

FIG. 35 is an oblique view of the femur, tibia, and bone pins of FIG. 32 with an 8-in-1 cut guide coupled to the distal anterior femoral bone pins, after making a distal femoral resection through the 8-in-1 cut guide;

FIG. 36 is an oblique view of the femur, tibia, and bone pins of FIG. 35 with the 8-in-1 cut guide coupled to the proximal anterior tibial bone pins, after making a proximal tibial resection through the 8-in-1 cut guide;

FIG. 37 is an oblique view of the femur, tibia, and bone pins of FIG. 36 with the 8-in-1 cut guide coupled to the distal femoral bone pins, after making anterior, anterior chamfer, posterior chamfer, and posterior resections through the 8-in-1 cut guide;

FIG. 38 is an oblique view of the 8-in-1 cut guide of FIG. 35;

FIG. 39 is another oblique view of the 8-in-1 cut guide of FIG. 38 from a different direction;

FIG. 40 is a front view of the 8-in-1 cut guide of FIG. 38;

FIG. 41 is a cross-sectional view of the 8-in-1 cut guide of FIG. 40, taken along section line 41-41;

FIG. 42 is an oblique view of the femur and tibia of FIG. 37 after removing the 8-in-1 cut guide and bone pins, with a tibial spacer inserted between the posterior femoral resection and the proximal tibial resection, and a bone pin inserted through the tibial spacer into a proximal aspect of the tibia;

FIG. 43 is an oblique view of the tibial spacer of FIG. 42;

FIG. 44 is an oblique view of the femur, tibia, tibial spacer, and pin of FIG. 42, with a tibial drill advancing over the bone pin;

FIG. 45 is an oblique view of the femur, tibia, tibial spacer, and pin of FIG. 42, with a tibial broach advancing over the bone pin;

FIG. 46 is an oblique view of the femur and support of FIG. 16, a femoral intramedullary drill guide positioned against a central distal anterior cortical portion of the femur, a femoral extension rod coupled to the femoral intramedullary drill guide and received in a target positioned over the center of the femoral head, the target coupled to the support;

FIG. 47 is an oblique view of the femoral intramedullary drill guide of FIG. 46;

FIG. 48 is an oblique view of the femur and femoral intramedullary drill guide of FIG. 46, with an intramedullary drill advanced through the femoral intramedullary drill guide partially into the intramedullary canal of the femur;

FIG. 49 is an oblique view of the femur of FIG. 48, with a T-handle and extended intramedullary rod inserted into the intramedullary canal of the femur;

FIG. 50 is an oblique view of the femur of FIG. 49, a tibia, foot bones, and a 6-in-1 pin guide advancing over an intramedullary pin to contact the femur and tibia, the tibia and foot bones secured in the foot holder of FIG. 16;

FIG. 51 is an oblique view of the intramedullary pin of FIG. 50;

FIG. 52 is a side view of the intramedullary pin of FIG. 51;

FIG. 53A is an oblique view of the femur, tibia, foot bones, 6-in-1 pin guide, intramedullary pin, and foot holder of FIG. 50, a tibial extension rod coupled to the 6-in-1 pin guide and received in a target of the foot holder; and FIG. 53B is an enlarged detail view of a distal portion of the femur, a proximal portion of the tibia, and a proximal portion of the 6-in-1 pin guide of FIG. 53A;

FIG. 54 is an oblique view of the femur, tibia, 6-in-1 pin guide, intramedullary pin, and foot holder of FIG. 53A, with a bone pin inserted through the 6-in-1 pin guide into the distal aspect of the femur, two bone pins inserted through the 6-in-1 pin guide into a distal anterior aspect of the femur, and two bone pins inserted through the 6-in-1 pin guide into a proximal anterior aspect of the tibia, for a total of 6 pins inserted into the femur and tibia;

FIG. 55 is an oblique view of the femur, tibia, and bone pins of FIG. 54 after removing the 6-in-1 pin guide;

FIG. 56 is an oblique view of a femur, tibia, fibula, and foot bones with a foot holder assembly, a femoral target, a femoral alignment assembly, and a cut guide assembly;

FIG. 57 is an oblique view of a portion of the femoral alignment assembly and the cut guide assembly of FIG. 56;

FIG. 58 is an oblique view of the portion of the femoral alignment assembly and a portion of the cut guide assembly of FIG. 57 from a different direction;

FIG. 59 is an exploded oblique view of the portions of the femoral alignment assembly and cut guide assembly of FIG. 58;

FIG. 60 is another exploded oblique view of the portions of the femoral alignment assembly and cut guide assembly of FIG. 58 from a different direction;

FIG. 61 is an exploded oblique view of a body, handle, base, thumbscrew, screw, and pin of the femoral alignment assembly of FIG. 59;

FIG. 62 is another exploded oblique view of the body, handle, base, thumbscrew, screw, and pin of FIG. 60 from a different direction;

FIG. 63 is an exploded oblique view of a femoral body, pin lock button, screw, and pin of the cut guide assembly of FIG. 59;

FIG. 64 is another exploded oblique view of the femoral body, pin lock button, screw, and pin of FIG. 63 from a different direction;

FIG. 65 is an exploded oblique view of a tibial body, size lock button, spring, and pins of the cut guide assembly of FIG. 59;

FIG. 66 is another exploded oblique view of the tibial body, size lock button, spring, and pins of FIG. 65 from a different direction;

FIG. 67 is an oblique view of a tibial cut guide of the cut guide assembly of FIG. 59;

FIG. 68 is another oblique view of the tibial cut guide of FIG. 67 from a different direction;

FIG. 69 is an oblique view of portions of the femur, tibia, fibula, femoral alignment assembly, and cut guide assembly of FIG. 56 with bone pins inserted through the cut guide assembly into the femur and tibia;

FIG. 70 is an oblique view of portions of the femur, tibia, cut guide assembly, and bone pins of FIG. 69 after making a distal femoral resection and a proximal tibial resection through the cut guide assembly;

FIG. 71 is an oblique view of portions of the femur, tibia, cut guide assembly, and some of the bone pins of FIG. 70 after pushing the cut guide assembly against the distal femoral resection and proximal anterior tibia;

FIG. 72 is an oblique view of portions of the femur, tibia, cut guide assembly, and bone pins of FIG. 71 after making anterior and posterior femoral resections through the cut guide assembly;

FIG. 73 is an oblique view of a distal end of a femur with a Whiteside's angle gage;

FIG. 74 is another oblique view of the distal end of the femur and Whiteside's angle gage of FIG. 73 from a different direction;

FIG. 75 is an oblique view of a femur, tibia, fibula, and foot bones coupled to a foot holder assembly, a cut guide assembly, and a tibial alignment rod assembly;

FIG. 76 is an enlarged detail view of a portion of FIG. 75 in the vicinity of the knee joint;

FIG. 77 is an oblique exploded view of a cut guide assembly of FIG. 75 separated into three part groups or sub-assemblies;

FIG. 78 is an oblique exploded view of a first femoral group of FIG. 77;

FIG. 79 is another oblique exploded view of the first femoral group of FIG. 78 from a different direction;

FIG. 80 is an oblique exploded view of a second femoral group of FIG. 77;

FIG. 81 is another oblique exploded view of the second femoral group of FIG. 80 from a different direction;

FIG. 82 is an oblique exploded view of a tibial group of FIG. 77;

FIG. 83 is another oblique exploded view of the tibial group of FIG. 82 from a different direction;

FIG. 84 is an oblique view of the femur, tibia, and cut guide assembly of FIG. 75 after making distal femoral and proximal tibial resections through the cut guide assembly;

FIG. 85 is an oblique view of the femur, tibia, and cut guide assembly of FIG. 84 after removing some bone pins, moving the cut guide assembly to abut the distal femoral resection, and inserting more bone pins through the cut guide assembly into the femur and tibia;

FIG. 86 is an oblique view of the femur, tibia, and cut guide assembly of FIG. 85 after making anterior and posterior femoral resections through the cut guide assembly;

FIG. 87 is an anterior view of a femur, showing longitudinal and kinematic lines of the femur;

FIGS. 88A and 88B are perspective views of a sizing guide according to one embodiment;

FIG. 89 is a perspective view showing the sizing guide of FIGS. 88A and 88B in place on the femur with anterior attachment and

FIG. 90 is an anterior view showing the sizing guide of FIGS. 88A and 88B on the femur, with the anterior attachment omitted.

### DETAILED DESCRIPTION

Exemplary embodiments of the technology will be best understood by reference to the drawings, wherein like parts are designated by like numerals throughout. It will be readily understood that the components of the technology, as generally described and illustrated in the figures herein, could be arranged and designed in a wide variety of different configurations. Thus, the following more detailed description of the embodiments of the apparatus, system, and method is not intended to limit the scope of the invention, as claimed, but is merely representative of exemplary embodiments of the technology.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together. The phrase “fluid communication” refers to two features that are connected such that a fluid within one feature is able to pass into the other feature.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

Standard medical planes of reference and descriptive terminology are employed in this specification. While these terms are commonly used to refer to the human body, certain terms are applicable to physical objects in general.

A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body. Proximal means toward the trunk of the body. Proximal may also mean toward a user or operator. Distal means away from the trunk. Distal may also mean away from a user or operator. Dorsal means toward the top of the foot. Plantar means toward the sole of the foot.

Standard terminology related to knee arthroplasty is employed in this specification with the ordinary and customary meanings. Varus means deviation of the distal part of the leg below the knee inward, resulting in a bowlegged appearance. Valgus means deviation of the distal part of the leg below the knee outward, resulting in a knock-kneed appearance.

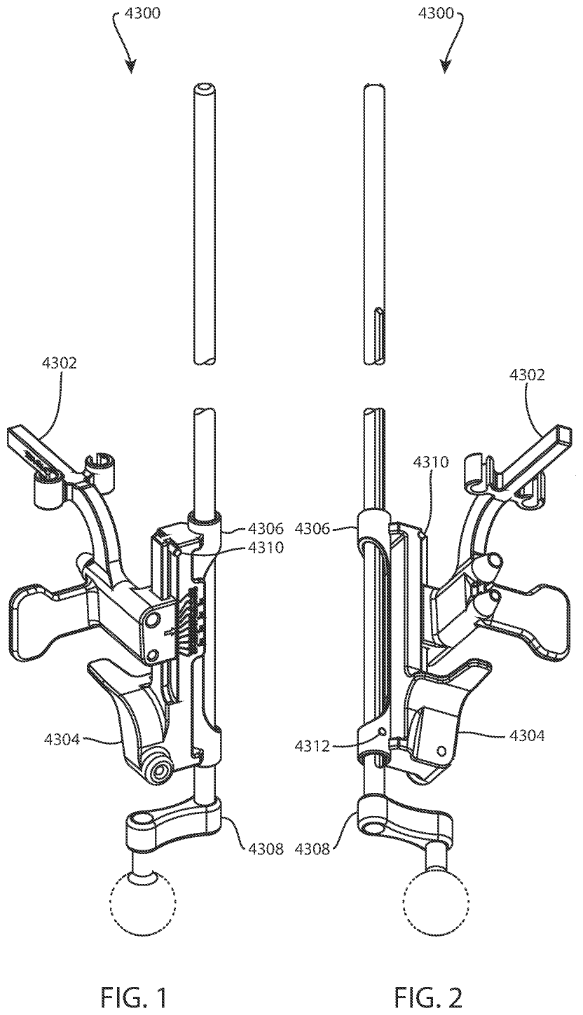

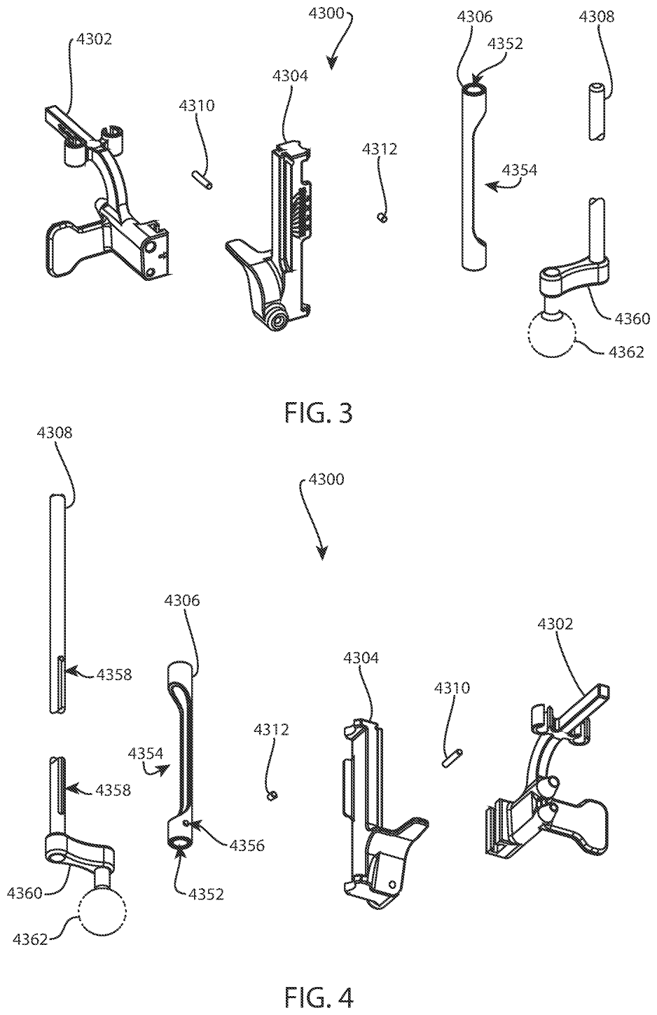

Referring to FIGS. 1-8, a multi-pin guide assembly 4300 may include a femoral multi-pin guide 4302 or femoral apparatus, a tibial pin guide 4304 or tibial apparatus, a tube 4306, a tibial alignment rod 4308, and pins 4310, 4312. This single instrument directly references the mechanical axis 202 of the leg and other patient anatomical features to place an array of 5 pins (or holes) that are subsequently referenced by cut guides to make all femoral and tibial resections for total knee arthroplasty. The array of pins or holes is placed in a highly coordinated manner by the single instrument, as opposed to conventional apparatus and methods where pins are placed at different stages of the procedure by different pin guides. This instrument references the mechanical axis 202 of the leg in a manner derived from the apparatus and methods set forth in U.S. patent application Ser. No. 15/630,555, which is incorporated herein in its entirety. More specifically, the multi-pin guide assembly 4300 couples to a base, a handle, a femoral alignment/extension rod, a femoral target, and a tibial target to align the apparatus with the mechanical axis 202 of the leg, as discussed below.

The femoral multi-pin guide 4302 may include an arm 4314 that extends superiorly and anteriorly, terminating in a free end 4316 which may engage a complementary socket of a handle, base, or other component, similar to socket 3624 of femoral pin guide assembly 3500. The arm 4314 may include a pair of tubes 4318, 4320 located on medial and lateral sides of the arm. The tubes 4318, 4320 include holes 4322, 4324 which extend parallel to each other along an anterior-posterior direction. The tubes 4318 may include longitudinal slots 4326, 4328 through the superior side walls of the tubes. The holes 4322, 4324 may receive pin sleeves, such as pin sleeve 3510 discussed above or pin sleeves 1515, 2515 disclosed in U.S. patent application Ser. No. 15/630,555, which is incorporated herein in its entirety. The holes 4322, 4324 preferably correspond to holes in a distal cut guide, as discussed below. The femoral multi-pin guide 4302 may include a femoral condyle paddle 4330 or plate at the inferior posterior end of the arm 4314. In use, the condyle paddle 4330 is placed in direct contact with a femoral condyle, thus it may be referred to as a femur-contacting feature. The condyle paddle 4330 may be a flat plate which extends along medial-lateral and anterior-posterior directions. Therefore, the condyle paddle 4330 may establish a plane that is parallel to the plane established by the holes 4322, 4324. The condyle paddle 4330 shown is for a medial femoral condyle, so it extends medially from the arm 4314. When the illustrated condyle paddle 4330 contacts the medial femoral condyle, the holes 4322, 4324 are positioned so that the distal cut guide will make an 8 mm cut. The same portion of the arm 4314 may include a protrusion 4332 which extends inferiorly from the junction with the condyle paddle 4330. The protrusion 4332 may include anterior and posterior holes 4334, 4336 which extend parallel to each other along a superior-inferior direction which may be parallel with a bone contacting surface of a base when viewed in a lateral view, or which may have a defined orientation relative to the bone contacting surface of the base, for example an acute angle in a lateral view. The direction of the holes 4334, 4336 may be parallel to the mechanical axis 202 of the leg in an anterior view. The anterior hole 4334 may be larger in diameter than the posterior hole. In the example shown, the anterior hole 4334 has a 5 mm diameter and the posterior hole 4336 has a 3.2 mm hole. The holes 4334, 4336, taken together, establish the rotational orientation of the four-in-one cut guide for the anterior, anterior chamfer, posterior chamfer, and posterior resections of the distal femur 100, as well as the depth and orientation of the anterior resection relative to the distal anterior femoral cortex immediately superior to the trochlear groove. An inferior portion of the protrusion 4332 may include a T-slot 4338 or dovetail slot which extends along an anterior-posterior direction along one medial or lateral side of the protrusion 4332.

The tibial pin guide 4304 may include a T-rail 4340 or dovetail rail which is complementary to the T-slot 4338 of the femoral multi-pin guide 4302. The T-rail may extend along an anterior-posterior direction relative to the femur 100. A transverse through hole 4342 may extend across the anterior end of the T-rail 4340 to receive the pin 4310. The tibial pin guide 4304 may include a tibial condyle paddle 4344 or plate which protrudes superiorly (relative to the femur 100) from a posterior portion of the tibial pin guide. In use, the condyle paddle 4344 is placed in direct contact with a tibial condyle or intercondylar area of the tibial plateau, thus it may be referred to as a tibia-contacting feature. The condyle paddle 4344 may be a flat plate which extends along medial-lateral and superior-inferior directions. Therefore, the condyle paddle 4344 may establish a plane that is perpendicular to the planes established by the holes 4322, 4324 and the femoral condyle paddle 4330. Alternatively, the condyle paddle 4344 may be at an acute angle relative to the planes established by the holes 4322, 4324 and the femoral condyle paddle 4330 in a lateral view. The tibial pin guide 4304 may include a hole 4346 which extends along a superior-inferior direction, parallel to the condyle paddle 4344 in a lateral view and parallel to the mechanical axis 202 of the leg in an anterior view. The hole 4346 is preferably spaced apart from the bone-contacting surface of the condyle paddle 4344 so that when the condyle paddle 4344 contacts the tibial plateau, the hole 4346 is positioned so that a tibial cut guide will make a proximal tibial resection at the desired distance from the posterior femoral resection, to accommodate the planned implant thickness, and in the same rotational alignment as the femoral cut guide. The tibial pin guide 4304 may include anterior and posterior brackets 4348, 4350 which couple to the tube 4306.

The tube 4306 includes a longitudinal through hole 4352. The tube 4306 may include a window 4354 which extends through one side wall in a central portion of the tube. The tube 4306 may include a hole 4356 which extends through one side wall of the tube near the posterior end to receive the pin 4312.

The tibial alignment rod 4308 is shown foreshortened in FIGS. 1-4 due to its long length. The tibial alignment rod 4308 may include a unilateral longitudinal groove 4358 which receives the pin 4312 to prevent rotation of the tibial alignment rod inside the tube 4306. Alternatively, the tube hole 4352 and tibial alignment rod 4308 may have complementary non-circular shapes, such as rectangle, square, or hexagonal. The tibial alignment rod 4308 may include a transverse arm 4360 extending from the distal end of the rod and terminating in a target-engaging finial 4362 which is a sphere in the example shown. The transverse arm 4360 positions the finial 4362 directly in line with the arm 4314 and holes 4334, 4336 of the femoral multi-pin guide 4302 and the hole 4346 of the tibial pin guide 4304 in an anterior-posterior direction relative to the femur 100.

The multi-pin guide assembly 4300 may be assembled by performing some or all of the following steps in any order.

Orienting the tibial pin guide 4304 relative to the femoral multi-pin guide 4302 as shown in FIG. 1, sliding the T-rail 4340 into the T-slot 4338, positioning the protrusion 4332 adjacent to the tibial condyle paddle 4344, and fixing the pin 4310 in the hole 4342 to make the tibial pin guide 4304 captive to the femoral multi-pin guide 4302.

Orienting the tube 4306 relative to the tibial pin guide 4304 as shown in FIG. 2 and fixing the tube to the brackets 4348, 4350.

Orienting the tibial alignment rod 4308 relative to the tube 4306 as shown in FIG. 2, sliding the rod into the hole 4352, inserting the pin 4312 through the hole 4356 and into the groove 4358, and fixing the pin 4312 in the hole 4356.

When the multi-pin guide assembly 4300 is fully assembled, the tibial pin guide 4304 is movable relative to the femoral multi-pin guide 4302 along an anterior-posterior direction (relative to the femur 100) established by the T-rail 4340 in the T-slot 4338. Unintentional disassembly is prevented by the pin 4310 extending across the T-rail 4340. Indicia 4364, 4366 on the femoral multi-pin guide 4302 and the tibial pin guide 4304 indicate to the user the femoral component size for a particular patient. The tibial alignment rod 4308 is movable relative to the tibial pin guide 4304 along an anterior-posterior direction (relative to the femur 100) established by the rod in the hole 4352.

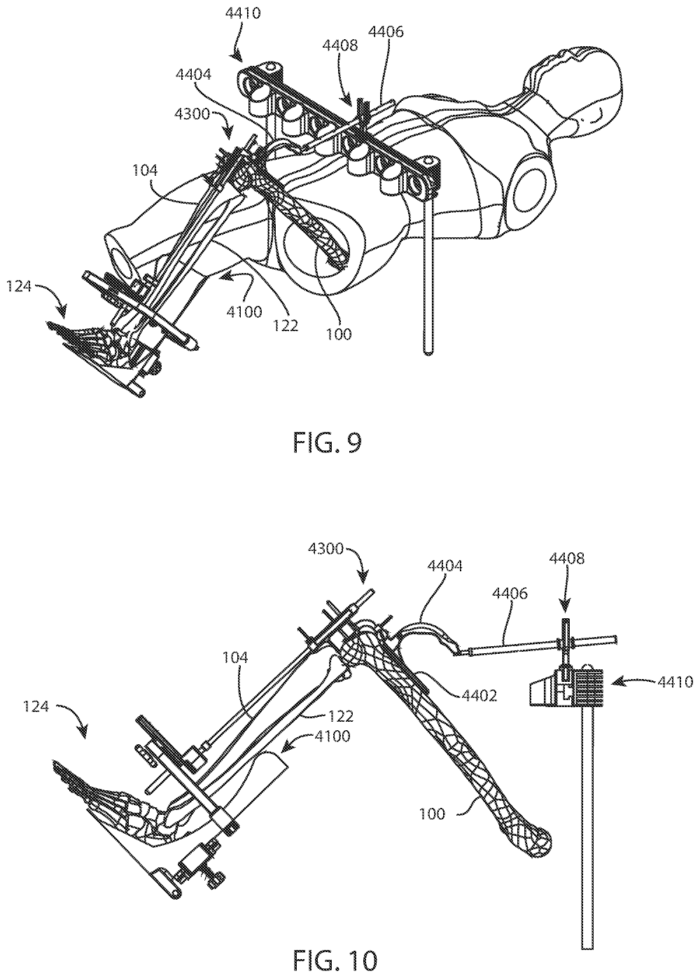

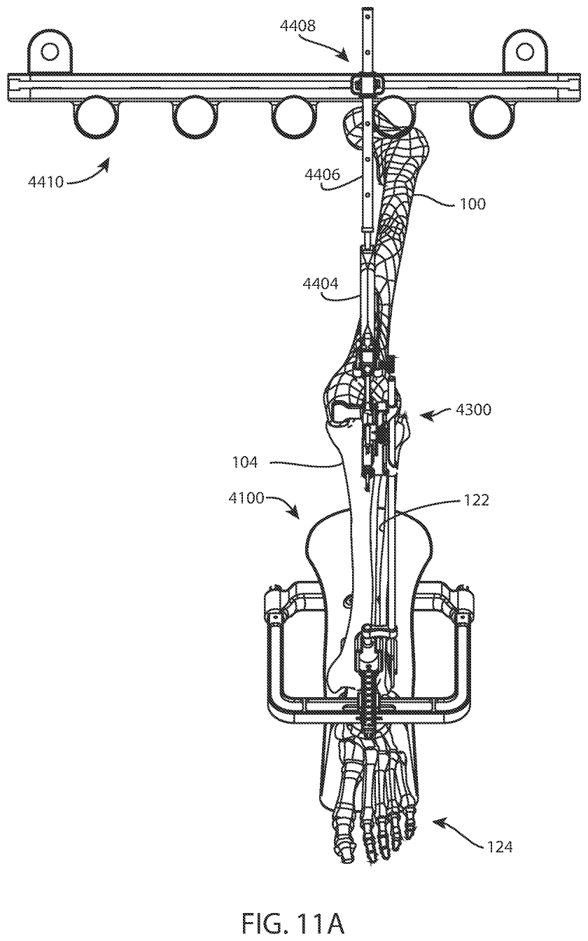

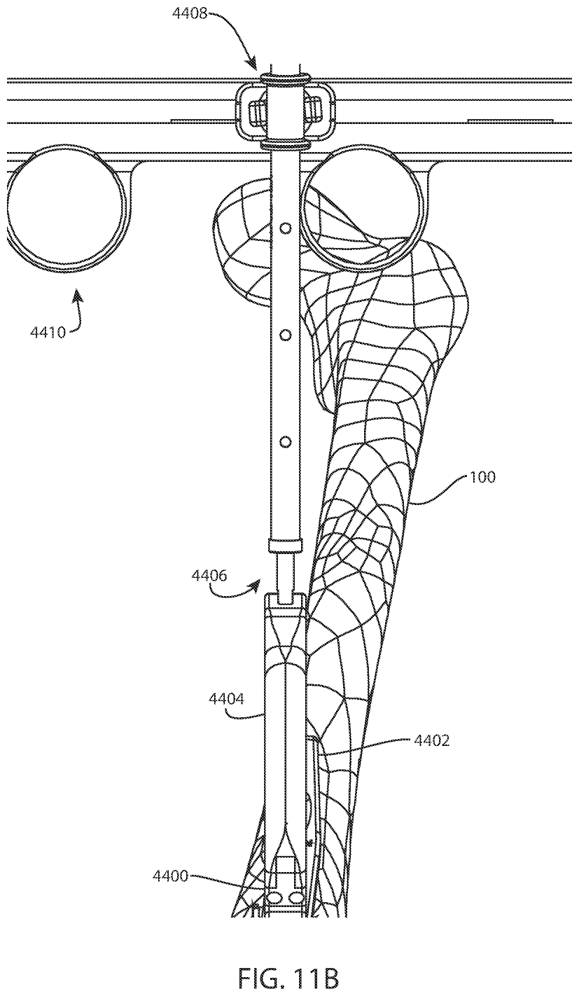

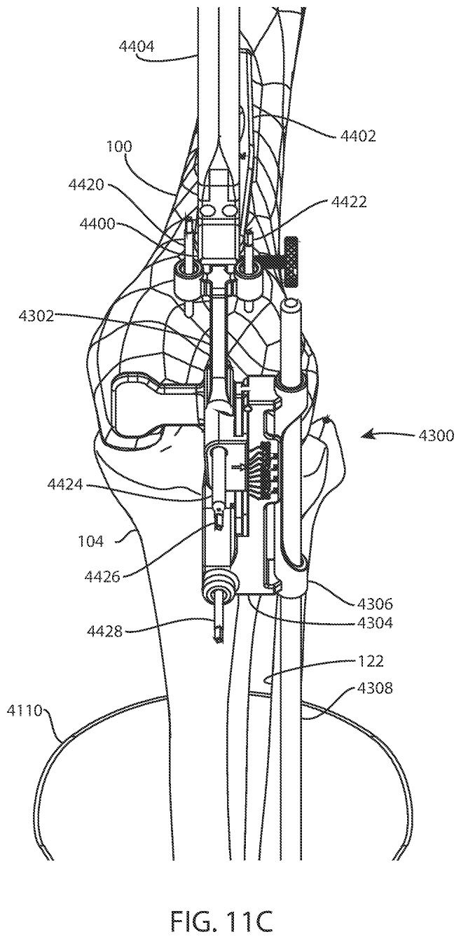

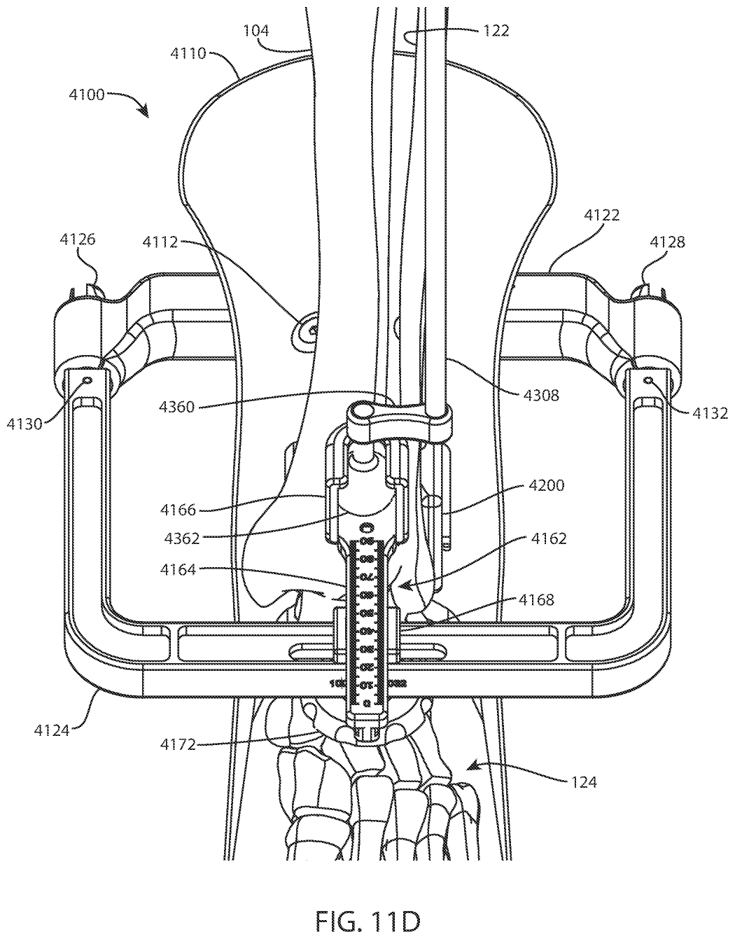

FIGS. 9-12 show the multi-pin guide assembly 4300 coupled to a body 4400, a base 4402, a handle 4404, a femoral extension rod 4406, a femoral target assembly 4408, a femoral support arm assembly 4410, and the foot holder assembly 4100. A femur 100, tibia 104, and fibula 122 are shown, along with foot bones 124. The knee is in 90 degrees of flexion. FIG. 9 shows selected regions of soft tissue of the head, torso, and right thigh for context.

The body 4400, base 4402, and handle 4404 may be comparable to portions of the femoral pin guide assembly 3500 or the trunnion 12, base 10, handle 26; base 302, handle 304; femoral riser 504, base 502; base 1502, handle 1516; and base 2502, handle 2516 disclosed in U.S. patent application Ser. No. 15/630,555. The body 4400, base 4402, and handle 4404 may be exchanged with these alternative apparatus.

The femoral extension rod 4406 may be similar to or identical to or exchanged with femoral extension rod 3524, or alignment rod 156 or femoral extension rods 306, 506, 1506, 2506 disclosed in U.S. patent application Ser. No. 15/630,555.

The femoral target assembly 4408 may be similar to or identical to or exchanged with target assemblies 818, 1818, 2818 disclosed in U.S. patent application Ser. No. 15/630,555.

The femoral support arm assembly 4410 may be similar to or identical to or exchanged with femoral support arm assemblies 786, 1786, 2786 disclosed in U.S. patent application Ser. No. 15/630,555. However, the assembly 4410 includes a bar having various openings and features conducive to organizing and storing surgical items.

The foot holder assembly 4100 may be similar to or identical to or exchanged with the foot holder assembly 3950 or foot holder assemblies 870, 1870, 2870 disclosed in U.S. patent application Ser. No. 15/630,555.

Referring to FIGS. 9-15B, a method of using the multi-pin guide assembly 4300 may include some or all of the following steps in any order.

Coupling the femoral multi-pin guide 4302 to a base and a femoral extension rod. This step may include inserting the free end 4316 of the femoral multi-pin guide arm 4314 into a socket like socket 3624 of femoral pin guide assembly 3500 and tightening a thumbscrew.

Aligning the femoral extension rod over the center of the femoral head and over the medial-lateral center of the distal femur 100. This step may include coupling the femoral extension rod to a femoral target which was previously centered relative to the femoral head.

Coupling the finial 4362 to a tibial target which was previously centered relative to the distal tibia 104 or medial-lateral center of the ankle. The knee may be in 90 degrees of flexion for this step and the following steps.

Centering the proximal tibia 104 relative to the hole 4346 of the tibial pin guide 4304 along a medial-lateral direction.

Contacting a distal femoral condyle with the condyle paddle 4330, contacting the tibial plateau with the condyle paddle 4344, and reading the femoral component size from the indicia 4364, 4366.

While maintaining the alignments of the preceding steps, driving pins 4420, 4422 through holes 4322, 4324 of the femoral multi-pin guide 4302; driving pins 4424, 4426 through holes 4334, 4336 of the femoral multi-pin guide 4302; and driving a pin 4428 through hole 4346 of the tibial pin guide 4304. This step may be preceded by a step of inserting pin sleeves in holes 4322, 4324 before driving pins 4420, 4422 through the holes. Pins 4424 and 4426 are parallel to each other, parallel to the femoral mechanical axis in an anterior view of the femur 100, parallel to (or in a defined orientation relative to) the bone-facing side of the base 4402 in a lateral view of the femur 100, and positioned along Whiteside's Line on the distal end of the femur 100. Pins 4420, 4422 are parallel to each other, perpendicular to pins 4424, 4426, and positioned along a line that is perpendicular to the femoral mechanical axis in an anterior view of the femur. Pin 4428 is parallel to pins 4424, 4426 and positioned along the tibial mechanical axis in an anterior view of the tibia 104.

Optionally removing the pin sleeves from holes 4322, 4324, if used. Optionally disengaging the femoral extension rod from the femoral target, if used. Disengaging the tibial alignment rod 4308 from the tibial target. Disengaging the femoral alignment rod, base, and multi-pin guide assembly 4300 from the operative site by pulling the apparatus along the direction established by the pins 4424, 4426, 4428. Preferably, the pins 4424, 4426, 4428 and their corresponding holes 4334, 4336, 4346 are mutually parallel. The pins 4420, 4422 exit holes 4322, 4324 via slots 4326, 4328. FIG. 13 illustrates the femur 100 and tibia 104 after this step is complete.

Removing pins 4424, 4426 and coupling a distal femoral cut guide 4412 to the anterior distal femur 100 using pins 4420, 4422. Holes 4425, 4427 remain in the femur 100 after pins 4424, 4426 are removed. FIG. 14 illustrates the femur 100 and tibia 104 after this step is complete.

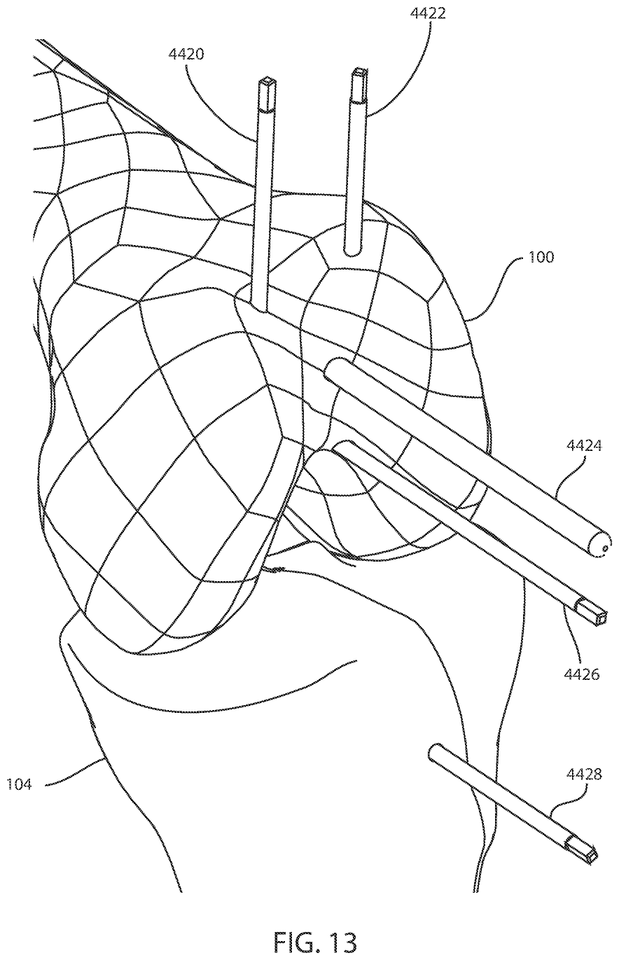

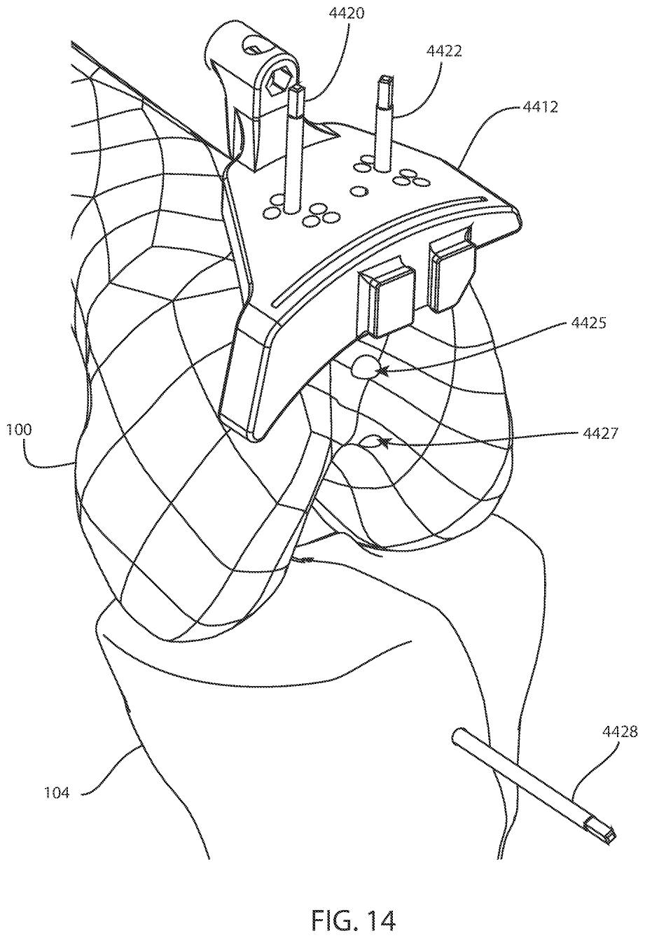

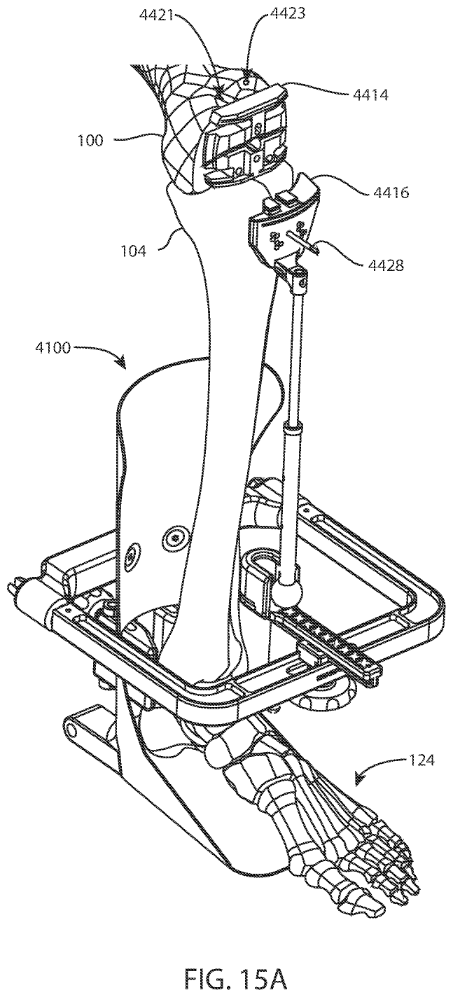

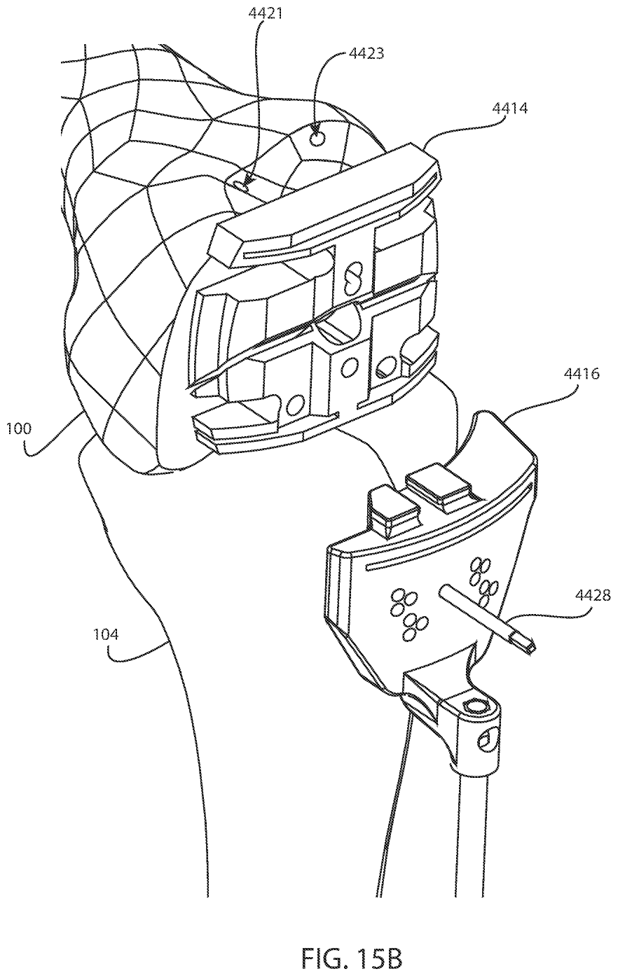

Making a distal femoral resection through the distal femoral cut guide 4412, removing pins 4420, 4422, coupling a femoral four-in-one cut guide 4414 to the distal femoral resection, coupling a tibial cut guide 4416 to the proximal anterior tibia 104 using pin 4428, and coupling the finial of a tibial extension rod to a tibial target which was previously centered relative to the distal tibia 104 or medial-lateral center of the ankle. Holes 4421, 4423 remain in the femur 100 after pins 4420, 4422 are removed. Coupling the femoral four-in-one cut guide 4414 to the distal femoral resection may include inserting two posts (not shown) on the bone-facing side of the cut guide 4414 into the holes 4425, 4427. This arrangement sets the rotation of the femoral four-in-one cut guide 4414 relative to Whiteside's Line. FIG. 15A illustrates the completion of this step and FIG. 15B provides a closeup view of the knee.

Additional steps may be performed, such as making the anterior, anterior chamfer, posterior, and posterior chamfer cuts and the proximal tibial cut, and removing the cut guides 4414, 4416 and pin 4428. At least the anterior femoral, posterior femoral, and proximal tibial cuts are made perpendicular to Whiteside's Line due to the orientation established by pins 4424, 4426 and holes 4425, 4427.

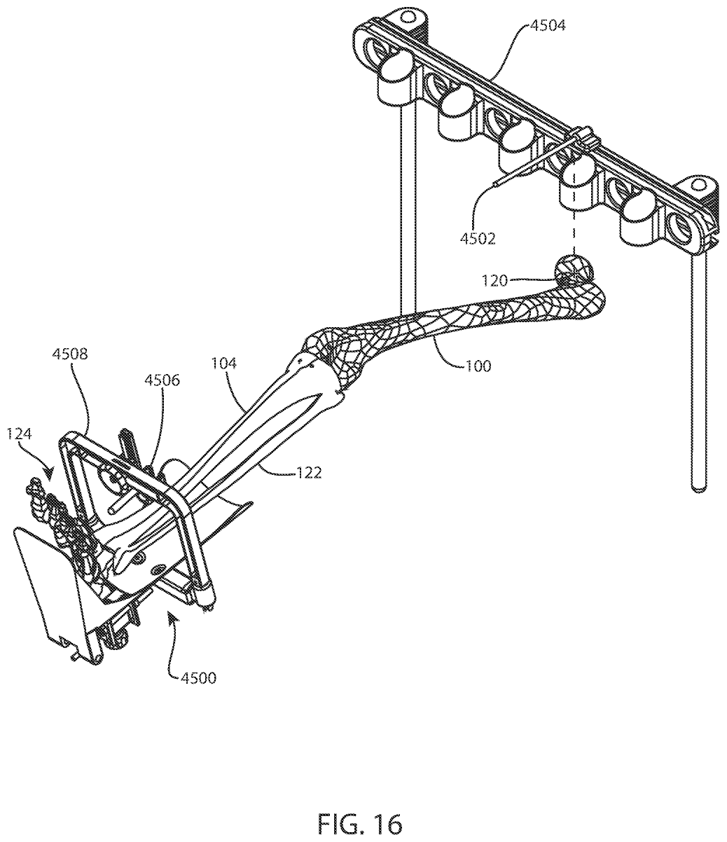

FIGS. 16-45 illustrate selected steps in a method of extramedullary knee arthroplasty, and related apparatus.

FIG. 16 shows a patient's femur 100, tibia 104, fibula 122, and foot bones 124. The tibia 104, fibula 122, and foot bones 124 are secured within a foot holder assembly 4500 that is in turn coupled to an operating table (not shown). A femoral head finder 4502 is coupled to a support apparatus 4504 and positioned over a rotational or spherical center 120 of a proximal head of the femur 100 of the patient. The support apparatus 4504 is in turn coupled to the operating table (not shown) by upright posts. The foot holder assembly 4500 may be the foot holder assembly 870, 1870, or 2870 of U.S. Pat. No. 10,568,650, or the foot holder assembly 3950 or 4100 of U.S. patent application Ser. No. 16/287,976. The femoral head finder 4502 may be the femoral head finder 918, 1918, or 2918 of U.S. Pat. No. 10,568,650. The support apparatus 4504 may be the femoral support arm assembly 786, 1786, or 2786 of U.S. Pat. No. 10,568,650, or the femoral support arm assembly 4410 of U.S. patent application Ser. No. 16/287,976.

A target 4506 is coupled to a bridge 4508 of the foot holder assembly 4500 and is positioned over a medial-lateral center of an ankle joint of the patient. Optionally, the target 4506 may be positioned over a medial-lateral center of another anatomical structure, such as the Achilles tendon, anterior tibial spine or crest, distal tibia 104, second toe, and the like. However, for brevity, the target 4506 will be referred to as an ankle center target 4506 in this application. The ankle center target 4506 may be the target 882, 1882, or 2882 of U.S. Pat. No. 10,568,650, or the target 3962 or 4162 of U.S. patent application Ser. No. 16/287,976.

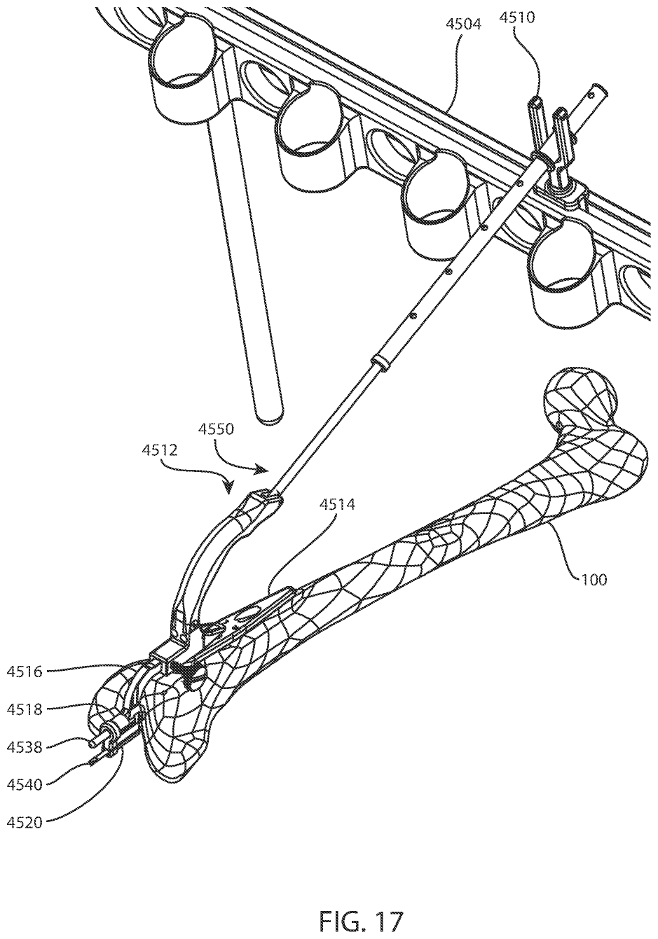

Referring to FIG. 17, the femoral head finder 4502 has been replaced by a target 4510 which is coupled to the support apparatus 4504 and is positioned over the femoral head center 120. The target 4510 will be referred to as a femoral head center target 4510 in this application. The femoral head center target 4510 may be the target 820, 1820, or 2820 of U.S. Pat. No. 10,568,650.

A femoral pin guide 4512 is positioned against a central distal anterior cortical portion of the femur 100. The femoral pin guide 4512 includes a base 4514 and an arm 4516. The arm 4516 includes at least one barrel 4518 or guide tube to guide a first bone pin 4538 from distal to proximal into a distal aspect of the femur 100. An optional additional barrel 4520 is shown, which is parallel to barrel 4518 and spaced apart from barrel 4518 along an anterior-posterior direction relative to the femur 100. A second bone pin 4540 is shown extending through barrel 4520. The femoral pin guide 4512 may be related to the pin guide assembly 1501 or 2501 of U.S. Pat. No. 10,568,650, or pin guide assembly 3500 or pin guide 4302, body 4400, base 4402, and handle 4404 of pin guide assembly 4300 of U.S. patent application Ser. No. 16/287,976. The first bone pin 4538 may be a drill bit (FIG. 20).

A distal end of a femoral extension rod 4550 is hinged to the femoral pin guide 4512 and a proximal end of the femoral extension rod 4550 is received in the femoral head center target 4510. The femoral extension rod 4550 may be considered as part of the femoral pin guide 4512, or as a separate apparatus connected to the femoral pin guide. The femoral extension rod may be related to the extension rods or alignment rods 156, 306, 313, 506, 511, 1506, 1511, 2506, 2511 of U.S. Pat. No. 10,568,650, or extension rods or alignment rods 3524, 4308, 4406 of U.S. patent application Ser. No. 16/287,976.

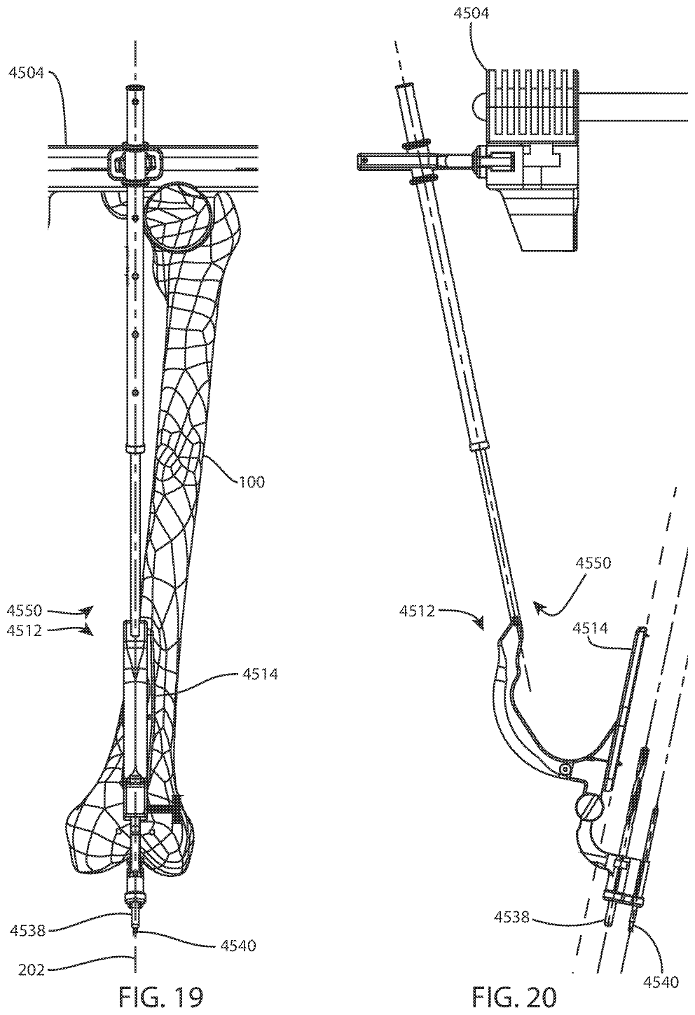

In an anterior view of the femur 100, and when positioned as described, the femoral extension rod 4550 lies along the mechanical axis 202 of the femur 100 (FIG. 19). First and/or second bone pins 4538, 4540 are inserted through the barrels 4518, 4520 or guide tubes of the femoral pin guide 4512 into a distal aspect of the femur 100. Preferably, the first and second bone pins 4538, 4540 are inserted into a central location along a medial-lateral direction, along Whiteside's Line, aligned with the femoral mechanical axis 202 in an anterior view of the femur 100, and aligned with or parallel to the bone-contacting surface of the base 4514 of the femoral pin guide 4512 in a lateral view of the femur 100 (FIG. 20). Preferably, the first and second bone pins 4538, 4540 are inserted into strong, dense subtrochlear bone anterior to the femoral intramedullary canal and posterior to the distal anterior femoral cortex. The femoral pin guide 4512 and femoral extension rod 4550 are removed after placing the first and/or second bone pins 4538, 4540.

In an optional step that may occur between FIGS. 17-20 and FIG. 21, the knee angle guide 2930 of U.S. Pat. No. 10,568,650 may be used to set the knee joint in 90° of flexion. The knee may also be set in 90° of flexion without using the knee angle guide 2930.

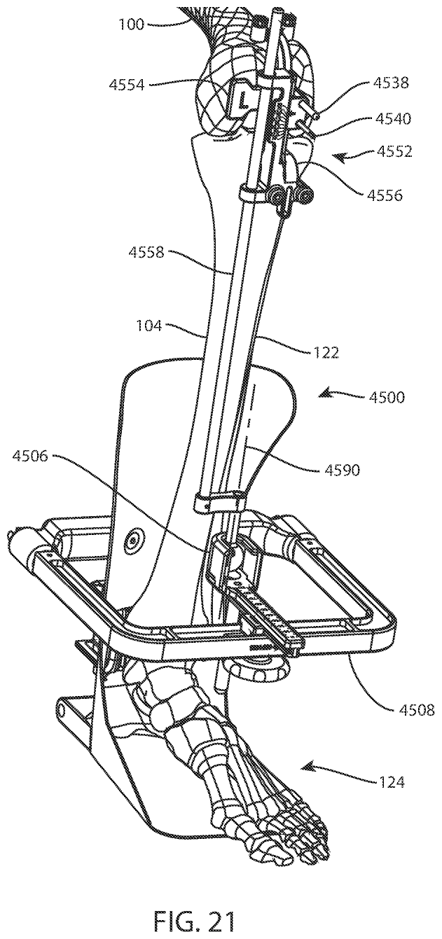

FIGS. 21-22 show the knee joint positioned in 90° of flexion. A 6-in-1 pin guide 4552 is coupled to the first and second bone pins 4538, 4540. The 6-in-1 pin guide 4552 may be related to the multi-pin guide assembly 4300, and may be referred to as a femoral-tibial or multi-pin guide. A femoral body 4554 or femoral apparatus of the 6-in-1 pin guide 4552 abuts a distal surface of a medial distal condyle of the femur 100. A tibial body 4556 or tibial apparatus of the 6-in-1 pin guide 4552 contacts a proximal surface of the tibial plateau. Preferably, contact occurs at the base of the tibial eminence, where an optional provisional tibial resection has been made. The tibial body 4556 of the 6-in-1 pin guide 4552 is movable relative to the femoral body 4554 along an anterior-posterior direction relative to the femur 100 by virtue of an interconnection between the femoral and tibial bodies 4554, 4556, discussed below. Preferably, the tibial body 4556 is movable relative to the femoral body 4554 only in linear translation along the anterior-posterior direction. The femoral and tibial bodies 4554, 4556 include indicia which indicate the corresponding femoral implant component size when the tibial body 4556 of the 6-in-1 pin guide 4552 contacts the proximal surface of the tibial plateau. A proximal end of a tibial alignment rod 4558 is coupled to the tibial body 4556 of the 6-in-1 pin guide 4552 and a distal end of the tibial alignment rod 4558 is received in the ankle center target 4506. The tibial alignment rod 4558 may be related to the extension rods or alignment rods 156, 306, 313, 506, 511, 1506, 1511, 2506, 2511of U.S. Pat. No. 10,568,650, or extension rods or alignment rods 3524, 4308, 4406 of U.S. patent application Ser. No. 16/287,976.

If the optional second bone pin 4540 is not present, the 6-in-1 pin guide 4552 and tibial alignment rod 4558 may pivot about the first bone pin 4538 as the distal end of the tibial alignment rod 4558 is placed in the ankle center target 4506. In an anterior view of the tibia 104, and when positioned as described, at least the distal portion of the tibial alignment rod 4558 lies along the mechanical axis 202 of the tibia 104. Alternatively, when the 6-in-1 pin guide 4552 is coupled to the first and second bone pins 4538, 4540, the 6-in-1 pin guide 4552 and tibial alignment rod 4558 may be prevented from pivoting about the bone pins. Instead, the proximal and/or distal ends of the tibia 104 may be oriented and positioned relative to the 6-in-1 pin guide 4552 and tibial alignment rod 4558 in order to engage the distal end of the tibial alignment rod 4558 in the ankle center target 4506.

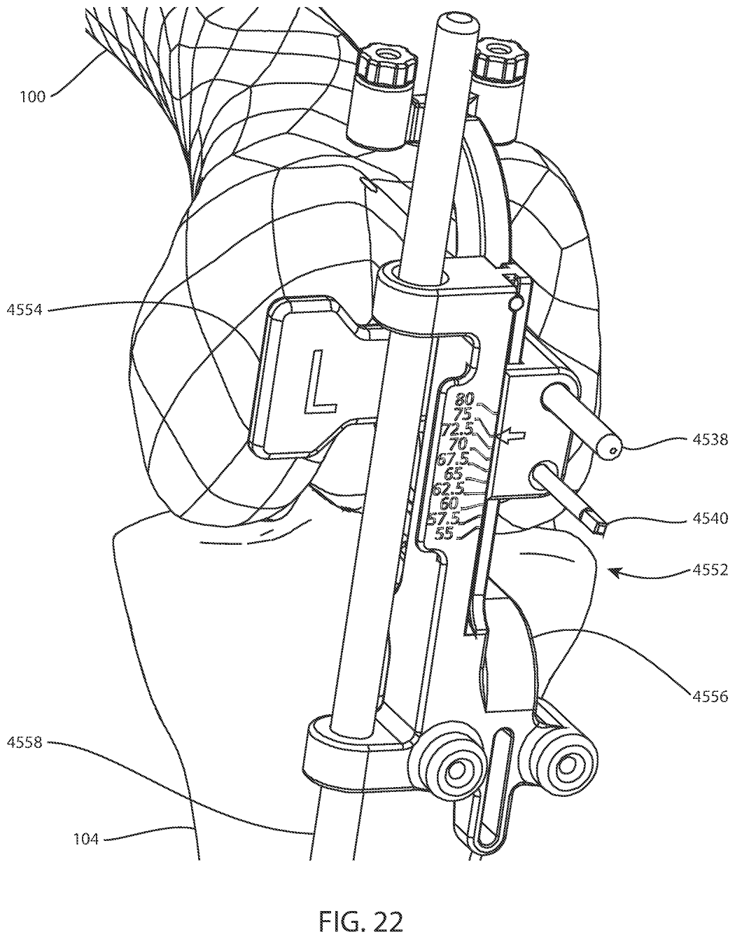

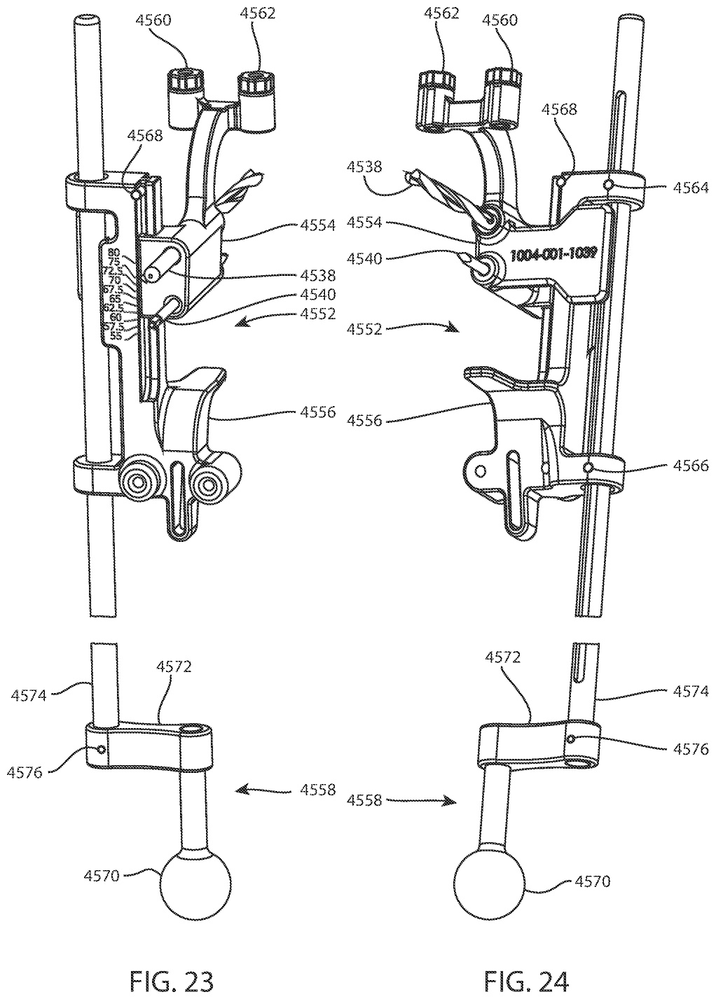

Referring to FIGS. 23-24, the 6-in-1 pin guide 4552 includes the femoral body 4554, removable pin sleeves 4560, 4562, the tibial body 4556, and fasteners, which in this example include three pins 4564, 4566, 4568. The tibial alignment rod 4558 may be considered as part of the 6-in-1 pin guide 4552, or as a separate apparatus connected to the 6-in-1 pin guide. The tibial alignment rod 4558 is shown foreshortened due to its length. The tibial alignment rod 4558 (which may also be referred to as a tibial extension rod) includes a distal target-engaging part 4570, an offset coupler 4572, a shaft 4574, and a fastener, which in this example is a pin 4576. The first and second bone pins 4538, 4540 are shown extending through the femoral body 4554.

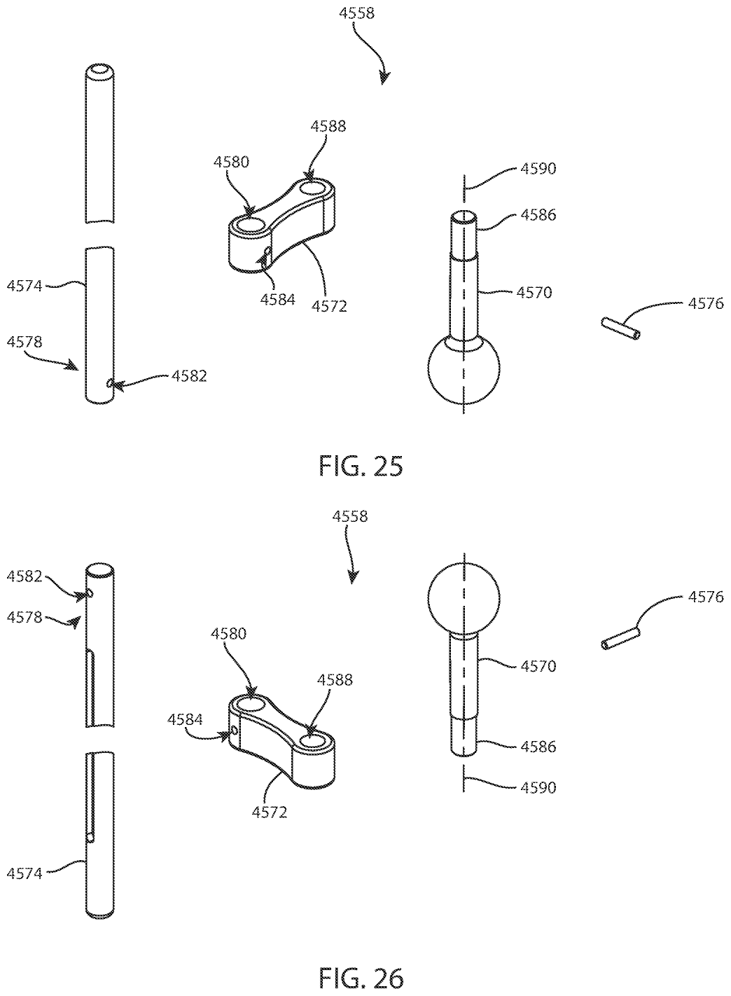

FIGS. 25-26 are exploded views of the tibial alignment rod 4558. The shaft 4574 is shown foreshortened due to its length. The distal end 4578 of the shaft 4574 is received in a first hole 4580 of the offset coupler 4572 and a pin 4576 is inserted through transverse holes 4582, 4584 in the shaft 4574 and offset coupler 4572 to fix the shaft and offset coupler together. The proximal end 4586 of the target-engaging part 4570 is received in a second hole 4588 of the offset coupler 4572. This interconnection may be secured with a pin, threads, snap fit, weld, or other means to fix the target-engaging part and offset coupler together. The offset coupler 4572 spaces the target-engaging part 4570 apart from the shaft 4574 along a medial-lateral direction to compensate for the medial-lateral distance between the ankle center target 4506 and the interconnection between the shaft 4574 and the femoral body 4554, so that in use, in an anterior view of the tibia, a central longitudinal axis 4590 of the target-engaging part 4570 lies along the mechanical axis 202 of the tibia 104.

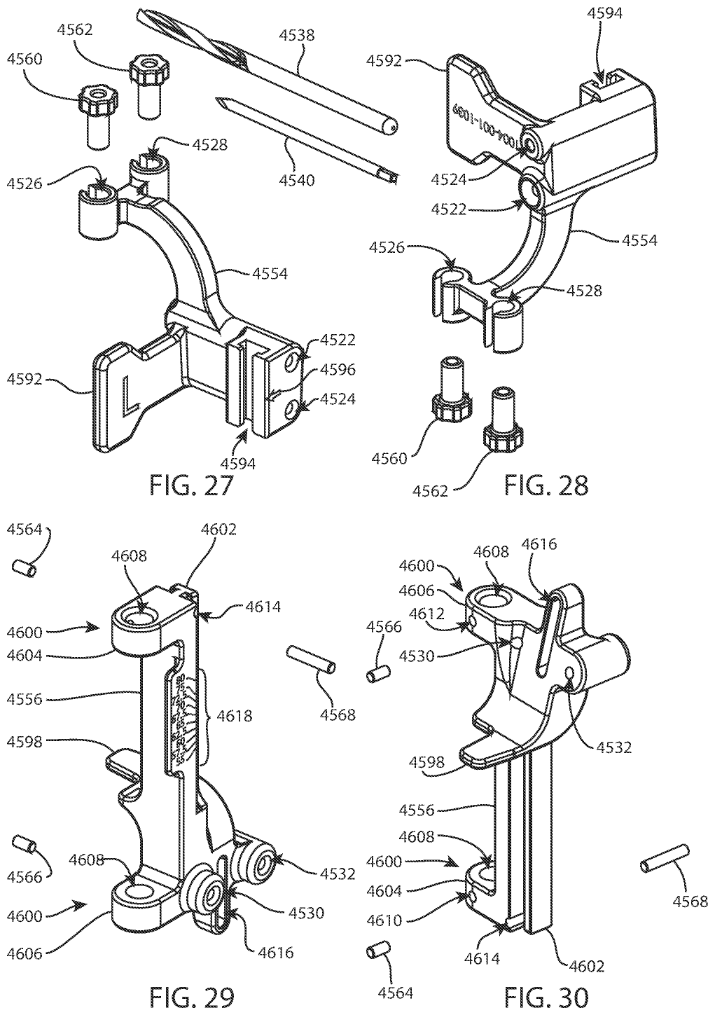

Referring to FIGS. 27-28, the femoral body 4554 of the 6-in-1 pin guide 4552 includes first and second barrels 4522, 4524 or guide tubes aimed along a distal-proximal direction into the distal aspect of the femur 100 in use, parallel, and spaced apart from each other along an anterior-posterior direction. The first barrel 4522 receives the first bone pin 4538, which is preferably an anterior barrel. The second barrel 4524 receives the second bone pin 4540, which is preferably a posterior barrel. The femoral body 4554 also includes third and fourth barrels 4526, 4528 aimed along an anterior-posterior direction into a distal anterior aspect of the femur 100 in use, parallel, and spaced apart from each other along a medial-lateral direction. The third barrel 4526 receives the pin sleeve 4560 and the fourth barrel 4528 receives the pin sleeve 4562. The four femoral barrels 4522, 4524, 4526, 4528 are in a fixed arrangement in this example since they are all included in a single unitary (monolithic) part. The femoral body 4554 also includes a distal femoral condyle paddle 4592 and a tibial body connection feature 4594. Referring briefly to FIG. 22, the distal femoral condyle paddle 4592 abuts a distal surface of a medial distal condyle of the femur 100 in use, thus it may be referred to as a femur-contacting feature. The third and fourth barrels 4526, 4528 are spaced apart from the condyle-contacting surface of the distal femoral condyle paddle 4592 along a distal-proximal direction relative to the femur 100. The tibial body connection feature 4594 in this example is a dovetail channel oriented along a proximal-distal direction relative to the tibia 104. The femoral body 4554 may include indicia 4596, such as the arrow shown, to cooperate with indicia on the tibial body to indicate the corresponding femoral implant component size.

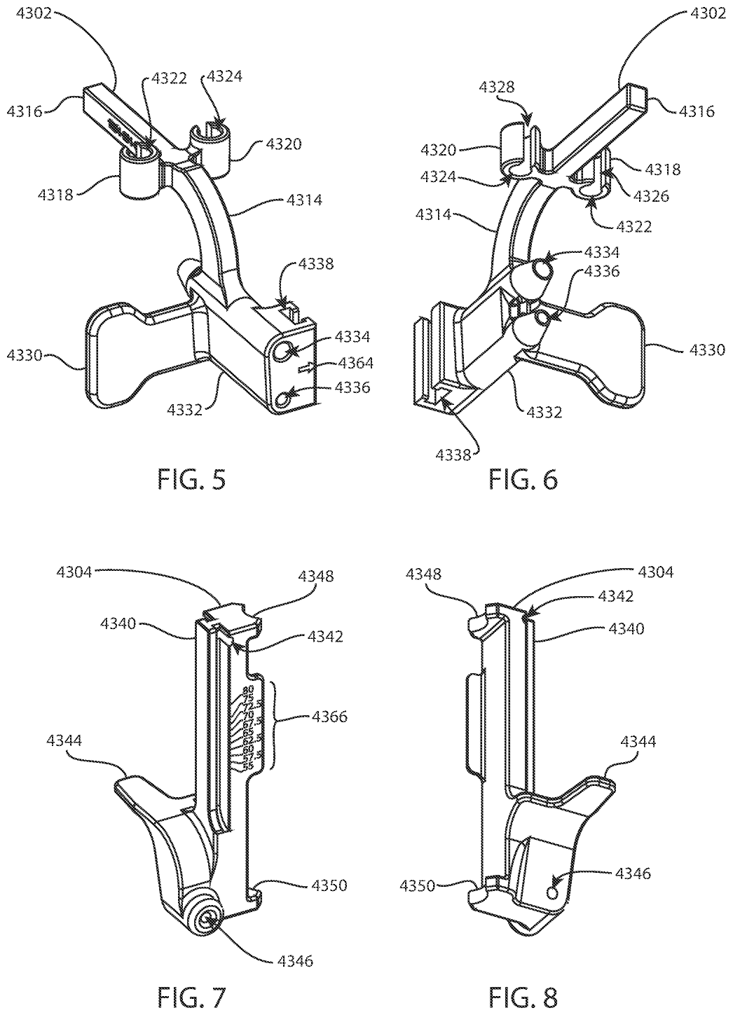

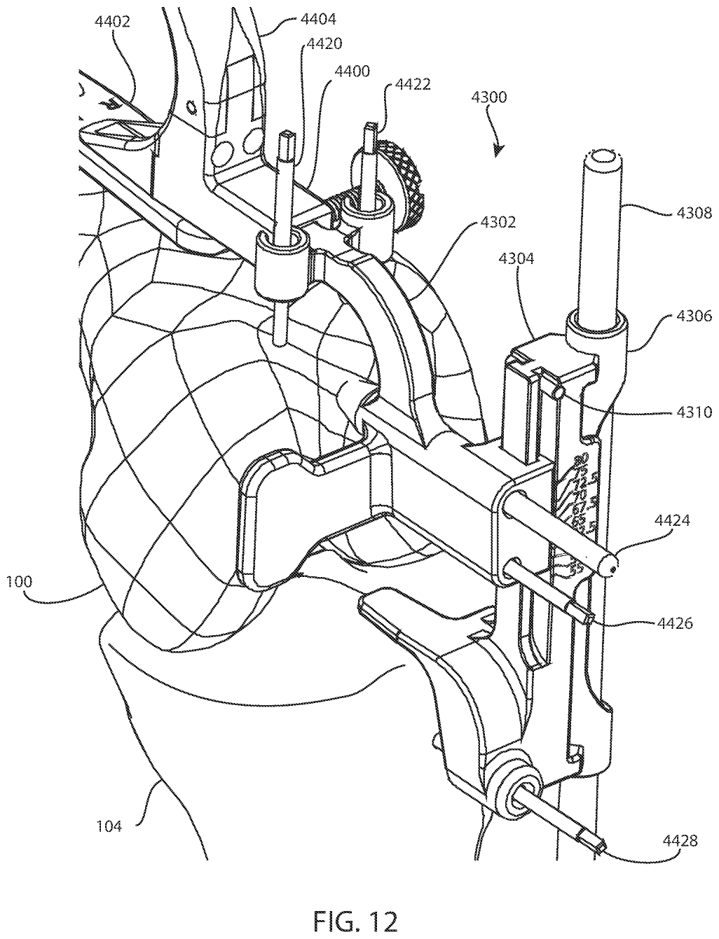

Referring to FIGS. 5-6 and 27-28, in an embodiment, the femoral body 4554 may be modified to connect to the body 4400, base 4402, and handle 4404 in a manner similar to the femoral multi-pin guide 4302, as seen best in FIG. 12.

Referring to FIGS. 29-30, the tibial body 4556 of the 6-in-1 pin guide 4552 includes fifth and sixth barrels 4530, 4532 aimed along a distal-proximal direction (relative to the femur 100) into an anterior aspect of the tibia 104 and spaced apart from each other along a medial-lateral direction. Because the knee joint is in 90° of flexion, the distal-proximal direction relative to the femur 100 equates to an anterior-posterior direction relative to the tibia 104. The two tibial barrels 4530, 4532 are in a fixed arrangement relative to each other in this example because they are both included in a single monolithic (unitary) part. However, they are free to translate along an anterior-posterior direction relative to the fixed arrangement of four femoral barrels 4522, 4524, 4526, 4528. The tibial body 4556 also includes a tibia-contacting feature 4598, a tibial alignment rod connection feature 4600, and a femoral body connection feature 4602. The tibia-contacting feature 4598 may be a paddle or arm or stalk that projects posteriorly relative to the tibia 104 in use to contact a proximal aspect of the tibia 104, the base of the tibial eminence, or a provisional proximal tibial resection. The tibial alignment rod connection feature 4600 in this example includes two bosses 4604, 4606 spaced apart along a proximal-distal direction relative to the tibia 104. A hole 4608 extends through the bosses 4604, 4606 and receives the shaft 4574 of the tibial alignment rod 4558. Fasteners (pins) 4564, 4566 are shown for holes 4610, 4612 in each boss 4604, 4606 to prevent unintentional disassembly while permitting free linear translation of the shaft 4574 in the hole 4608. The femoral body connection feature 4602 in this example is a dovetail rail that is complementary to the dovetail channel 4594 of the femoral body 4554 and oriented along a proximal-distal direction relative to the tibia 104. A fastener 4568 may be inserted into hole 4614 to prevent unintentional disassembly while permitting free linear translation of the rail 4602 in the channel 4594. The tibial body 4556 may include a central longitudinal slot 4616. The tibial body 4556 may include indicia 4618, such as the array of lines and numerals shown, to cooperate with indicia 4596 on the femoral body 4554 to indicate the corresponding femoral implant component size in use.

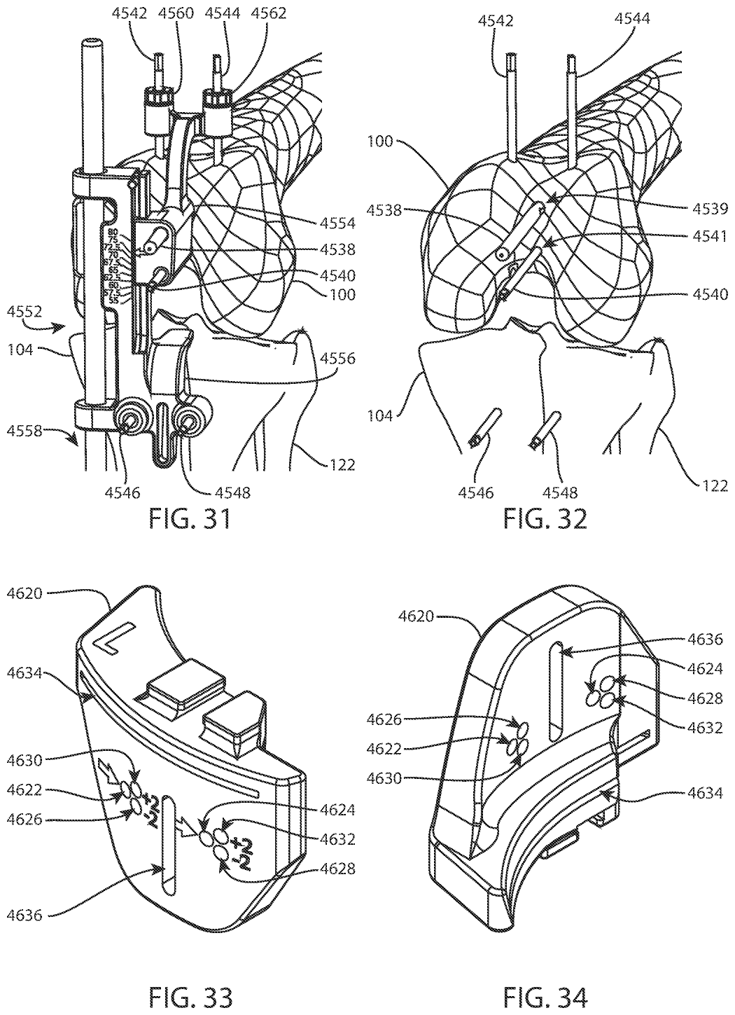

FIG. 31 shows the knee joint, 6-in-1 pin guide 4552, first and second bone pins 4538, 4540, and tibial alignment rod 4558 of FIGS. 21-22. The first and second bone pins 4538, 4540 have been inserted through the first and second barrels 4522, 4524 of the femoral body 4554. Third and fourth bone pins 4542, 4544 have been inserted through the pin sleeves 4560, 4562 in the third and fourth barrels 4526, 4528 of the femoral body 4554, and fifth and sixth bone pins 4546, 4548 have been inserted through the fifth and sixth barrels 4530, 4532 of the tibial body 4556. These 6 bone pins all extend through a single apparatus, the 6-in-1 pin guide 4552, while the guide is aligned with respect to the femoral mechanical axis 202, the tibial mechanical axis 202, the distal aspect of the medial femoral condyle, Whiteside's Line, and the proximal aspect of the tibia 104 at the base of the tibial eminence.

FIG. 32 shows the knee joint and bone pins 4538, 4540, 4542, 4544, 4546, 4548 of FIG. 31 after removing the 6-in-1 pin guide 4552 and tibial alignment rod 4558. Pins 4538, 4540 are parallel to each other, parallel to the femoral mechanical axis in an anterior view of the femur 100, parallel to (or in a defined orientation relative to) the bone-facing side of the base 4514 in a lateral view of the femur 100, and positioned along Whiteside's Line on the distal end of the femur 100. Pins 4542, 4544 are parallel to each other, perpendicular to pins 4538, 4540, and positioned along a line that is perpendicular to the femoral mechanical axis in an anterior view of the femur. Pins 4546, 4548 are parallel to each other and to pins 4538, 4540 (or at a defined slope), and positioned along a line that is perpendicular to the tibial mechanical axis in an anterior view of the tibia 104.

FIGS. 33-34 show oblique views of a cut guide 4620. The cut guide 4620 includes holes 4622, 4624 which receive the third and fourth bone pins 4542, 4544 or the fifth and sixth bone pins 4546, 4548. The example shown also includes holes 4626, 4628 which are offset −2 mm from holes 4622, 4624, and holes 4630, 4632 which are offset +2 mm from holes 4622, 4624. The cut guide includes a saw slot 4634 for making the distal femoral resection 206 or the proximal tibial resection 210. The cut guide may include a central longitudinal slot 4636. The cut guide 4620 may be related to the cut guide 4412 of U.S. patent application Ser. No. 16/287,976.

The cut guide 4620 may be coupled to the third and fourth bone pins 4542, 4544 so that a distal femoral resection 206 may be made through the saw slot 4634, optionally with the first and second bone pins 4538, 4540 temporarily removed; and moved to the fifth and sixth 4546, 4548 so that a proximal tibial resection 210 may be made through the saw slot 4634.

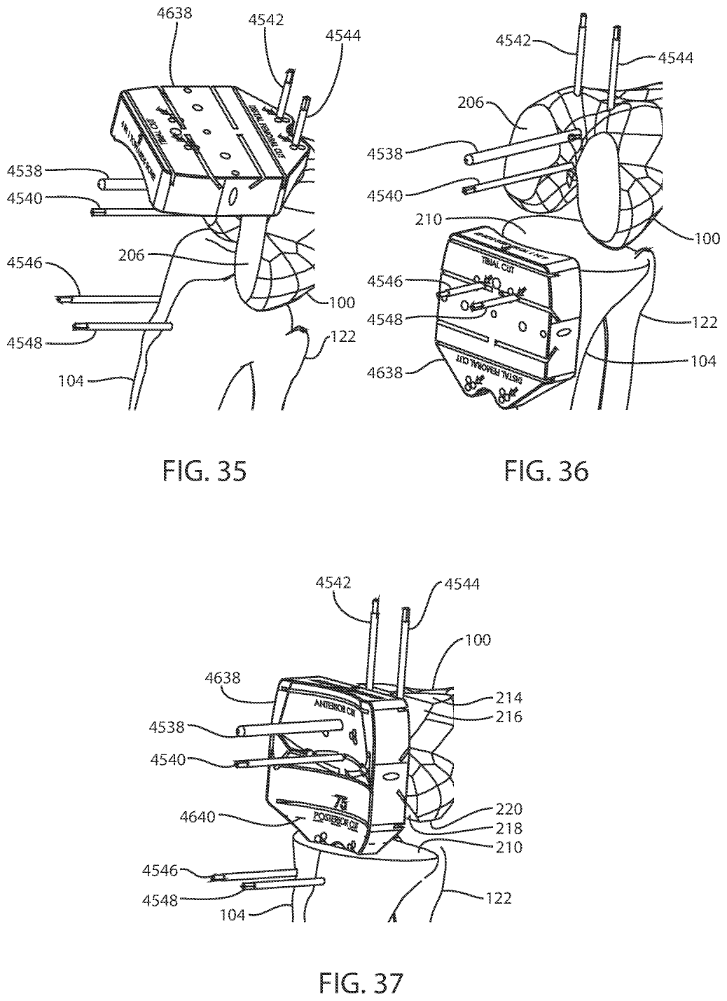

FIG. 35 shows the knee joint and bone pins of FIG. 32 with an 8-in-1 cut guide 4638 coupled to the third and fourth bone pins 4542, 4544. A distal femoral resection 206 has been made through the 8-in-1 cut guide 4638.

FIG. 36 shows the 8-in-1 cut guide 4638 moved to the fifth and sixth bone pins 4546, 4548. A proximal tibial resection 210 has been made through the 8-in-1 cut guide 4638.

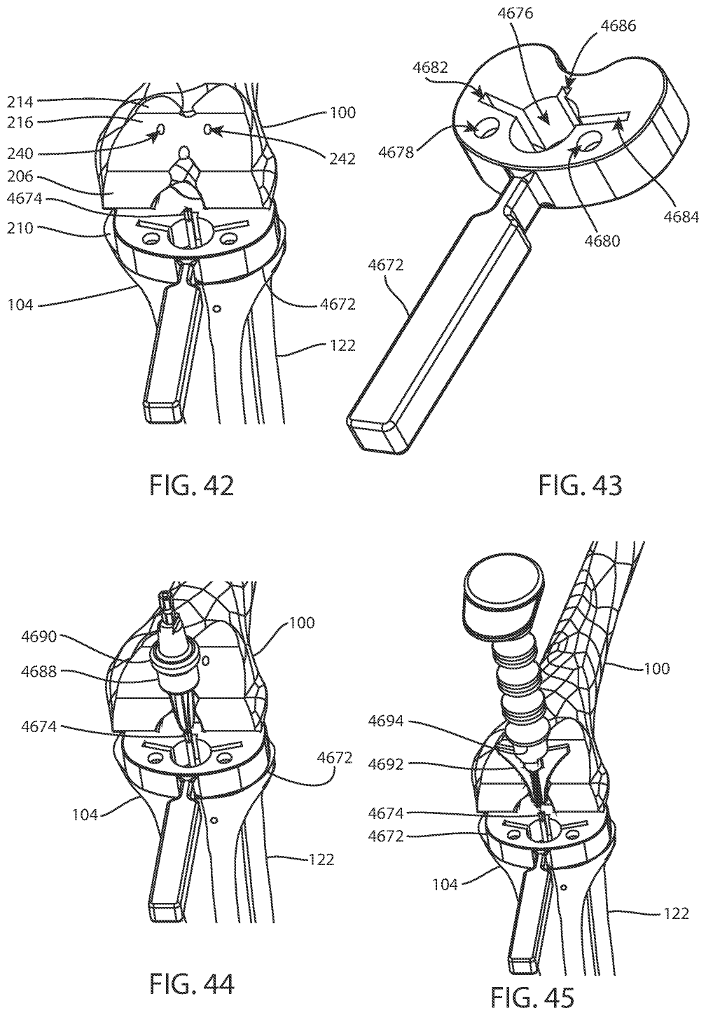

FIG. 37 shows the 8-in-1 cut guide 4638 moved to the first and second bone pins 4538, 4540. The third and fourth bone pins 4542, 4544 are shown remaining in position in the distal anterior femur 100, but optionally, they may be removed. An anterior femoral resection 214, anterior chamfer resection 216, posterior chamfer resection 218, and posterior femoral resection 220 have been made through the 8-in-1 cut guide 4638. Optionally, a drill may be used through the 8-in-1 cut guide 4638 to drill a pair of holes 240, 242 (FIG. 42) to receive pegs of a femoral implant component. Thus, the 8-in-1 cut guide 4638 may be used to make 1) distal femoral resection 206, 2) proximal tibial resection 210, 3) anterior femoral resection 214, 4) anterior chamfer resection 216, 5) posterior chamfer resection 218, 6) posterior femoral resection 220, 7) first peg hole 240, and 8) second peg hole 242. While two peg holes are discussed in this example, any number of peg holes may be present, and the 8-in-1 cut guide 4638 may include slots or holes to prepare the bone to receive other implant bone-engaging features. In this position, a portion of the 8-in-1 cut guide 4638 may interfere with the proximal tibia 104. In an optional design shown by dashed line 4640, a portion of the 8-in-1 cut guide 4638 may be a separate piece that can be removed for this step. At least the proximal tibial resection 210, anterior femoral resection 214, and posterior femoral resection 220 are made perpendicular to Whiteside's Line due to the orientation established by pins 4538, 4540.

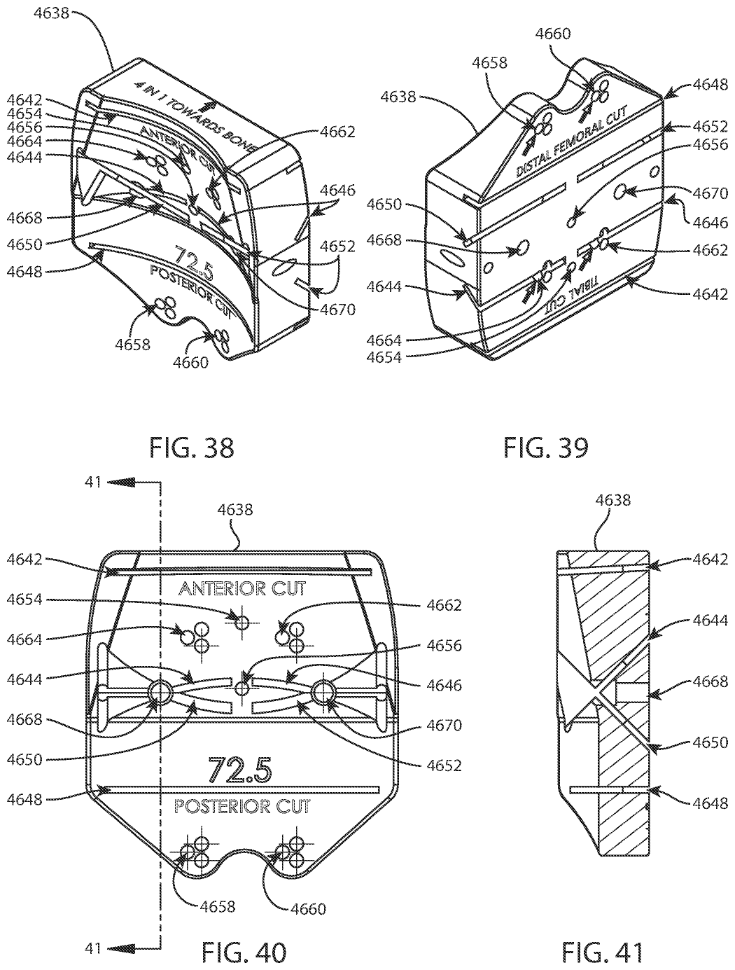

Referring to FIGS. 38-41, the 8-in-1 cut guide 4638 includes saw slot 4642 for the anterior femoral resection 214 and the proximal tibial resection 210; saw slots 4644, 4646 for the anterior chamfer resection 216; saw slot 4648 for the distal femoral resection 206 and the posterior femoral resection 220; and saw slots 4650, 4652 for the posterior chamfer resection 218. The 8-in-1 cut guide 4638 includes labels for the anterior femoral, distal femoral, posterior femoral, and proximal tibial saw slots in this example. The 8-in-1 cut guide 4638 also includes holes to receive the first through sixth bone pins discussed above. Holes 4654, 4656 receive the first and second bone pins 4538, 4540. Holes 4658, 4660 receive the third and fourth bone pins 4542, 4544. Holes 4662, 4664 receive the fifth and sixth bone pins 4546, 4548. The arrays of holes for the distal femoral resection 206 (third and fourth bone pins 4542, 4544) and proximal tibial resection 210 (fifth and sixth bone pins 4546, 4548) include extra hole pairs to provide some adjustability to the depth of the corresponding resection. The 8-in-1 cut guide 4638 also includes bilateral holes 4668, 4670 through which a drill may be actuated to prepare the first and second peg holes 240, 242.

FIG. 42 shows the knee joint of FIG. 37 after removing the 8-in-1 cut guide 4638 and bone pins 4538, 4540, 4542, 4544, 4546, 4548. A tibial spacer 4672 has been inserted between the posterior femoral resection 220 and the proximal tibial resection 210, with the knee joint in 90° of flexion. A seventh bone pin 4674 has been inserted through the tibial spacer 4672 along a proximal-distal direction relative to the tibia 104 into the proximal side of the tibia 104. An optional separate pin guide (not shown) may be temporarily inserted into the large central aperture of the tibial spacer 4672 to provide a close-fitting pin hole through which the seventh bone pin 4674 can be driven for more accurate placement.

FIG. 43 shows the tibial spacer 4672. The enlarged working end of the tibial spacer 4672 includes a large central aperture 4676 flanked by two smaller holes 4678, 4680, which may include counterbores at each end. This example also includes slots 4682, 4684, 4686 which intersect the central aperture 272. All of these features may extend through the working end.

FIG. 44 shows the knee joint, tibial spacer 4672, and seventh bone pin 4674 of FIG. 42. A tibial drill 4688 or reamer is shown oriented to be passed over the seventh bone pin 4674, through the tibial spacer 4672, and into the proximal tibia 104 to prepare for a tibial baseplate stem or peg. The tibial drill 4688 may be cannulated as shown, to pass over the seventh bone pin 4674. The tibial drill 4688 includes a flange 4690 or collar which contacts the tibial spacer 4672 to stop the drill at a predetermined depth within the tibia 104.

FIG. 45 shows the knee joint, tibial spacer 4672, and seventh bone pin 4674 of FIG. 44. The tibial drill 4688 has been removed. A tibial punch 4692 or broach is shown oriented to be passed over the seventh bone pin 4674, through the tibial spacer 4672, and into the proximal tibia 104 to prepare for tibial baseplate keels or ribs. The tibial punch 4692 may be cannulated to pass over the seventh bone pin 4674. Optionally, the seventh bone pin 4674 may be removed for this step. The tibial punch 4692 includes a platform 4694 or shoulder which contacts the tibial spacer 4672 to stop the punch at a predetermined depth within the tibia 104.

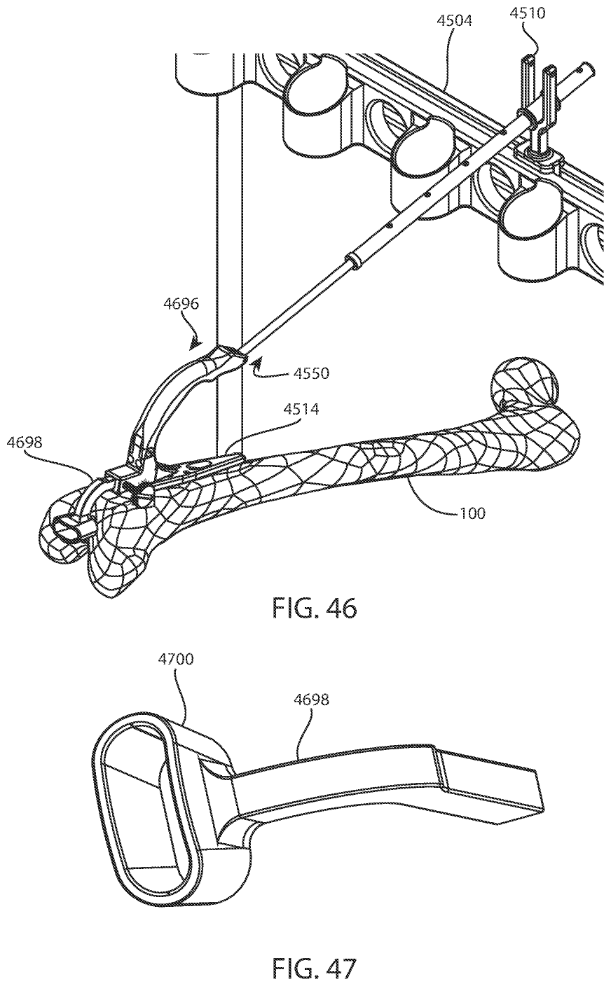

FIGS. 46-55 illustrate selected steps in a method of intramedullary knee arthroplasty, and related apparatus. These steps may occur after the step shown in FIG. 16, and may occur instead of the steps shown in FIGS. 17-32. The steps shown in FIGS. 33-45 may occur after the step shown in FIG. 55, optionally with apparatus modified as discussed below.

Referring to FIG. 46, a femoral intramedullary drill guide 4696 is positioned against a central distal anterior cortical portion of the femur 100. The femoral intramedullary drill guide 4696 includes the base 4514 and an arm 4698. The femoral intramedullary drill guide 4696 may be related to the pin guide assembly 1501 or 2501 of U.S. Pat. No. 10,568,650, or pin guide assembly 3500 or pin guide 4302, body 4400, base 4402, and handle 4404 of pin guide assembly 4300 of U.S. patent application Ser. No. 16/287976, or femoral pin guide 4512. In one example, the femoral intramedullary drill guide 4696 may be identical to the femoral pin guide 4512, except that the arm 4698 is included instead of the arm 4516. The distal end of the femoral extension rod 4550 is hinged to the femoral intramedullary drill guide 4696 and the proximal end of the femoral extension rod 4550 is received in the femoral head center target 4510. In an anterior view of the femur 100, and when positioned as described, the femoral extension rod 4550 lies along the mechanical axis 202 of the femur 100.

Referring to FIG. 47, the arm 4698 includes at least one barrel 4700 or guide tube to guide an intramedullary drill 4702 from distal to proximal into a distal aspect of the femur 100 and on into an intramedullary canal of the femur 100. The barrel 4700 may have a circular or non-circular opening through which the intramedullary drill 4702 passes. In the example shown, the barrel 4700 has a non-circular opening that is oval in a distal view of the femur 100 and tapers inwardly from distal to proximal.

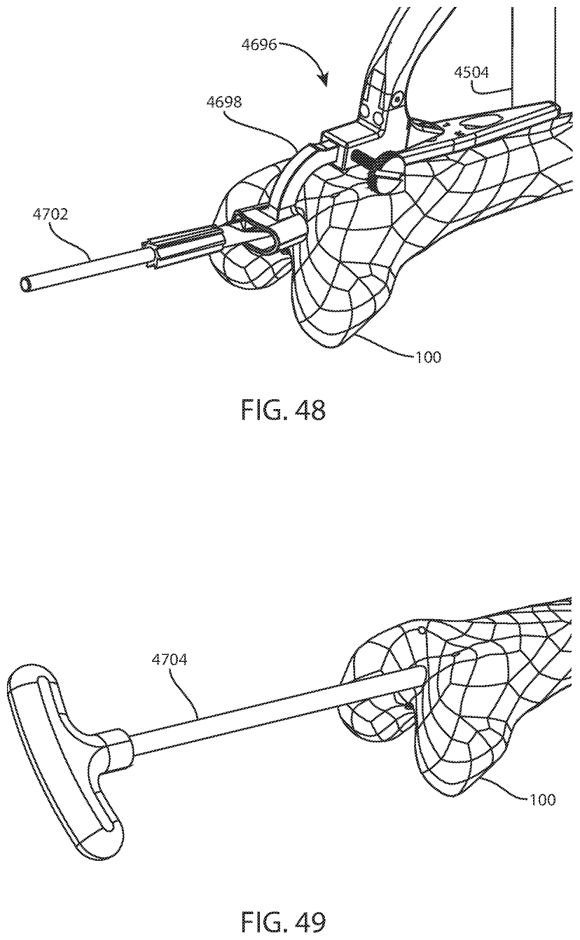

FIG. 48 shows the femur 100 and femoral intramedullary drill guide 4696 of FIG. 46. The intramedullary drill 4702 has been inserted through the barrel 4700 or guide tube of the femoral intramedullary drill guide 4696 along a distal-proximal direction into a distal aspect of the femur 100 in line with the intramedullary canal of the femur 100. Preferably, the intramedullary drill 4702 is inserted into a central location within the intramedullary canal along a medial-lateral direction, aligned with the femoral anatomical axis in an anterior view of the femur 100, and aligned with the bone-contacting surface of the base part of the femoral intramedullary drill guide 4696 4696 in a lateral view of the femur 100. The intramedullary drill 4702 and femoral intramedullary drill guide 4696 may be removed at the conclusion of this step.

Referring to FIG. 49, a longer intramedullary rod 4704 with T-handle has been inserted into the intramedullary canal of the femur. The intramedullary rod 4704 has a smaller outside diameter than the trailing end of the cutting portion of the intramedullary drill 4702. This provides space for intramedullary contents to extrude distally between the intramedullary rod 4704 and the distal portion of the hole made by the intramedullary drill 4702.

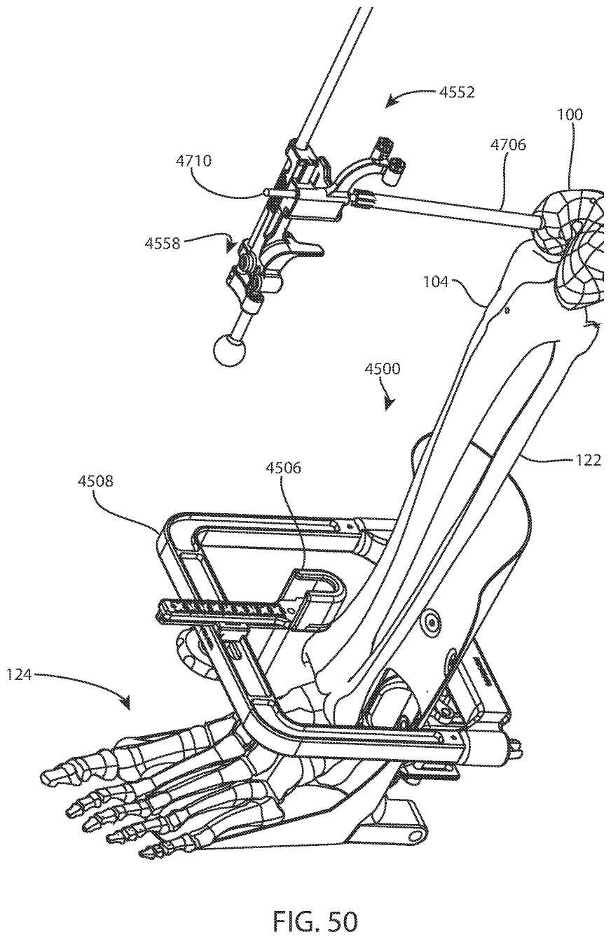

FIG. 50 shows the 6-in-1 pin guide 4552 coupled to an intramedullary pin 4706, which is positioned to be inserted into the prepared intramedullary canal. A distal portion 4710 of the intramedullary pin 4706 has been inserted into the first barrel 4522 of the femoral body 4554 of the 6-in-1 pin guide. The 6-in-1 pin guide 4552 may be referred to as a femoral-tibial or multi-pin guide. The 6-in-1 pin guide 4552 may be related to the multi-pin guide assembly 4300. It may be preferable to modify at least the femoral body 4554 to move the first barrel 4522 posteriorly to correspond to the location of the intramedullary canal versus the subtrochlear location discussed above, which is anterior to the intramedullary canal and posterior to the distal anterior femoral cortex. One of skill in the art will appreciate that other apparatus disclosed herein may benefit from similar modifications, such as apparatus that connects to the intramedullary pin 4706 instead of the first bone pin 4538. The tibial alignment rod 4558 is coupled to the 6-in-1 pin guide 4552 and is shown in a proximally-retracted position.

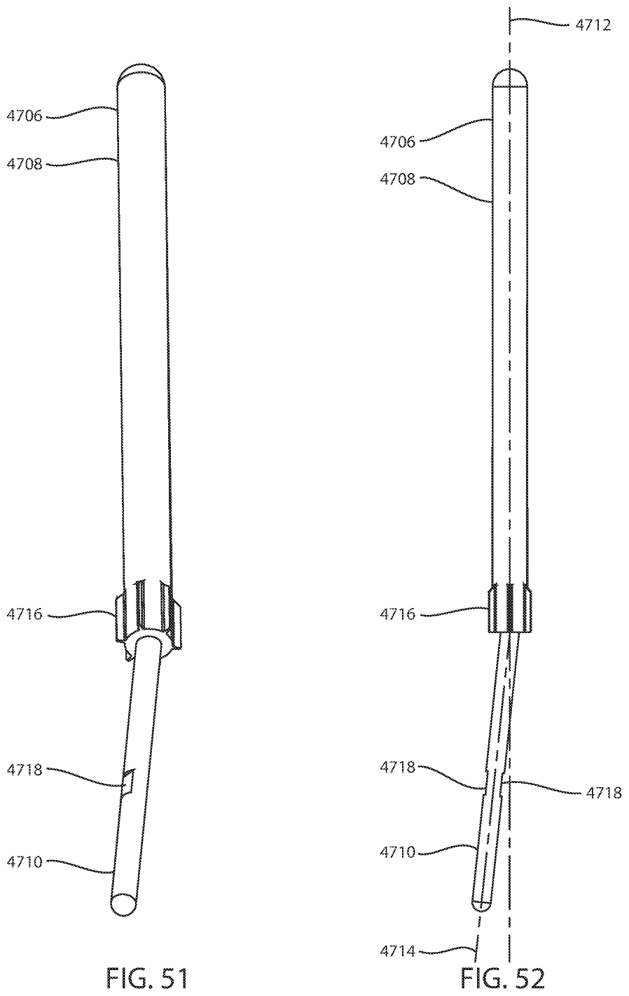

FIGS. 51-52 show the intramedullary pin 4706. The intramedullary pin 4706 includes a proximal portion 4708 and a distal portion 4710. The proximal portion 4708 is for insertion into the prepared intramedullary canal of the femur 100. The distal portion 4710 extends distally out of the femur 100 and is received in the first barrel 4522 of the 6-in-1 pin guide 4552. The proximal portion 4708 is angled relative to the distal portion 4710. The angle corresponds to the angle between the femoral mechanical axis 202 and the femoral anatomical axis (or the femoral intramedullary canal axis), in an anterior view of the femur 100. The central longitudinal axis 4712 of the proximal portion 4708 represents the femoral anatomical axis in use, and the central longitudinal axis 4714 of the distal portion 4710 represents the femoral mechanical axis 202 in use. In the example shown, the angle between the axes 4712, 4714 is 5°, although angles between 2° and 12° are contemplated. The proximal portion 4708 is longer than the distal portion 4710 and has a larger outside diameter. Where the proximal and distal portions 4708, 4710 meet, the proximal portion 4708 includes a rotation-resisting portion 4716 with a circular array of radially-projecting fins. In use, the rotation-resisting portion 4716 engages the distal mouth of the prepared intramedullary canal to prevent the intramedullary pin 4706 from rotating within the canal. The distal portion 4710 may include one or more flats 4718 to orient the intramedullary pin 4706 relative to the 6-in-1 pin guide 4552. The example shown has bilateral flats 4718 centrally located along the distal-proximal length of the distal portion 4710. Accordingly, the first barrel 4522 of the femoral body 4554 of the 6-in-1 pin guide 4552 may benefit from modification to include one or more flat-engaging features, as may other apparatus disclosed herein, such as apparatus that connects to the intramedullary pin 4706 instead of the first bone pin 4538. In this situation, the 6-in-1 pin guide 4552 may be prevented from pivoting about the distal portion 4710 of the intramedullary pin 4706.

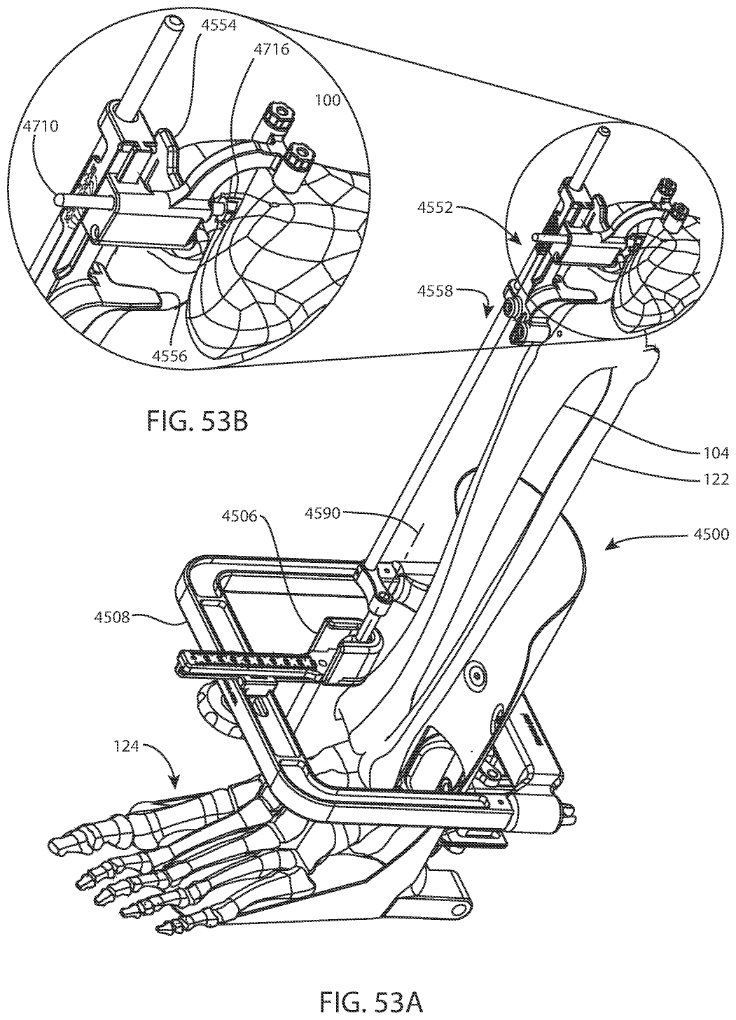

In FIGS. 53A-B, the 6-in-1 pin guide 4552, tibial alignment rod 4558, and intramedullary pin 4706 have advanced proximally relative to the femur 100 so that the proximal portion 4708 of the intramedullary pin 4706 is in the prepared intramedullary canal, the rotation-resisting portion 4716 is engaged with the distal mouth of the prepared intramedullary canal, the distal portion 4710 is received in the first barrel 4522 of the 6-in-1 pin guide 4552, the femoral body 4554 of the 6-in-1 pin guide 4552 abuts a distal surface of a medial condyle of the femur 100, and the tibial body 4556 of the 6-in-1 pin guide 4552 contacts a proximal surface of a tibial plateau. The tibial alignment rod 4558 has been extended distally and the target-engaging part 4570 has been received in the ankle center target 4506. The proximal and/or distal ends of the tibia 104 may be oriented and positioned relative to the 6-in-1 pin guide 4552 and tibial alignment rod 4558 to accomplish this. In an anterior view of the tibia 104, the central longitudinal axis 4590 of the target-engaging part 4570 lies along the mechanical axis 202 of the tibia 104.

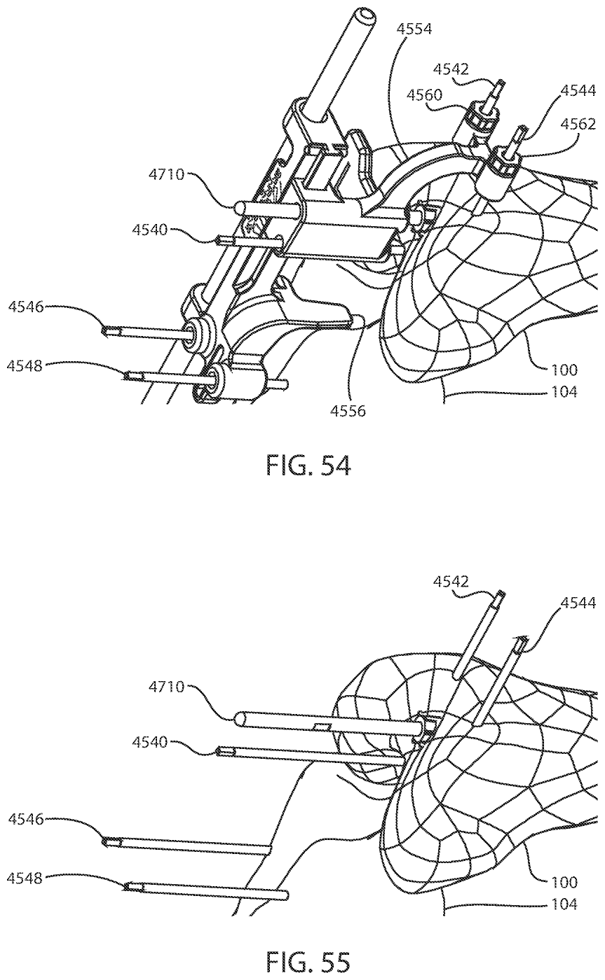

Referring to FIG. 54, the second bone pin 4540 has been inserted through the second barrel 4524 into the distal aspect of the femur 100. The third and fourth bone pins 4542, 4544 have been inserted through the pin sleeves 4560, 4562 in the third and fourth barrels 4526, 4528 into the distal anterior aspect of the femur 100. The fifth and sixth bone pins 4546, 4548 have been inserted through the fifth and sixth barrels 4530, 4532 into the proximal anterior aspect of the tibia 104. Thus, a total of 6 pins are inserted through the 6-in-1 pin guide 4552 into the femur 100 and tibia 104, considering the distal portion 4710 of the intramedullary pin 4706 as one of the pins. The 6-in-1 pin guide 4552 and tibial alignment rod 4558 may be removed at the conclusion of this step.

FIG. 55 shows the knee joint, intramedullary pin 4706, and bone pins 4540, 4542, 4544, 4546, 4548 of FIG. 354 after removing the 6-in-1 pin guide 4552 and tibial alignment rod 4558. The step shown in FIG. 55 is similar to the step shown in FIG. 32.

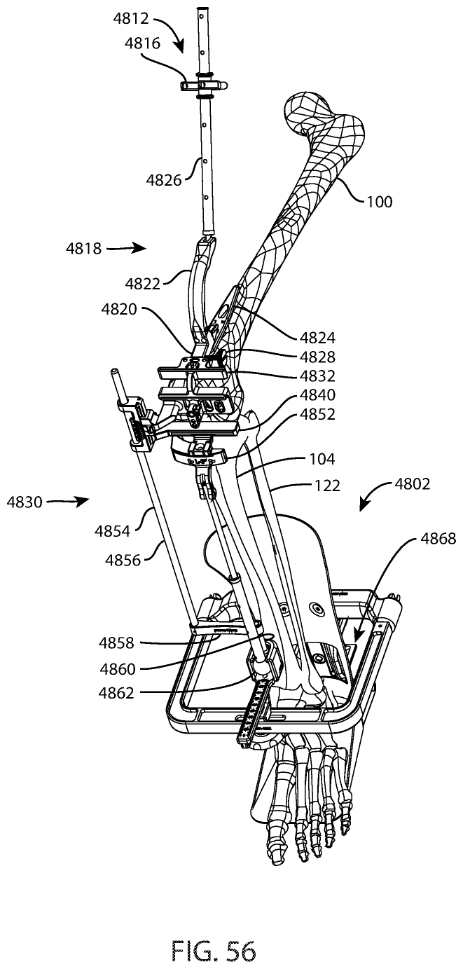

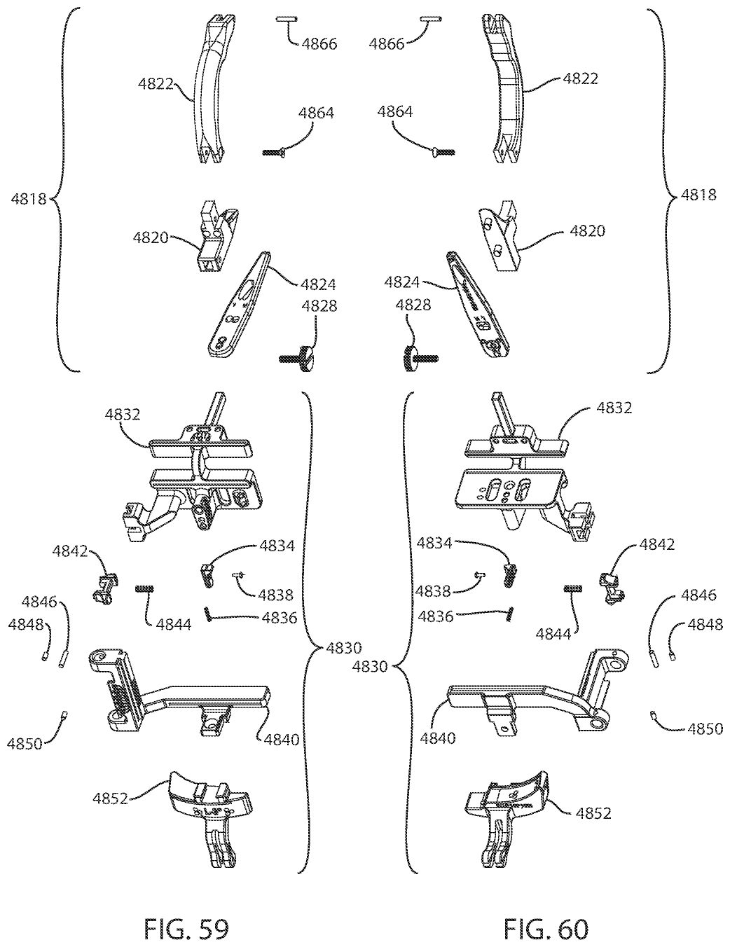

FIGS. 56-72 illustrate selected steps in a method of making femoral and tibial resections for knee arthroplasty, and related apparatus.

Referring to FIG. 56, a knee joint includes a femur 100, a tibia 104, and a fibula 122. Associated soft tissues are omitted for clarity so that the bones are visible. The knee joint is shown in 90 degrees of flexion. The tibia 104 and fibula 122 are secured in a foot holder assembly 4802. A cut guide assembly 4830 is coupled to the foot holder assembly 4802 so that it extends along the anterior side of the tibia 104 and the distal and anterior sides of the femur 100. A femoral alignment assembly 4818 is coupled to the cut guide assembly 4830, a central distal anterior cortical surface of the femur 100, and a femoral target 4816 so that the femoral alignment assembly extends along the anterior side of the femur.

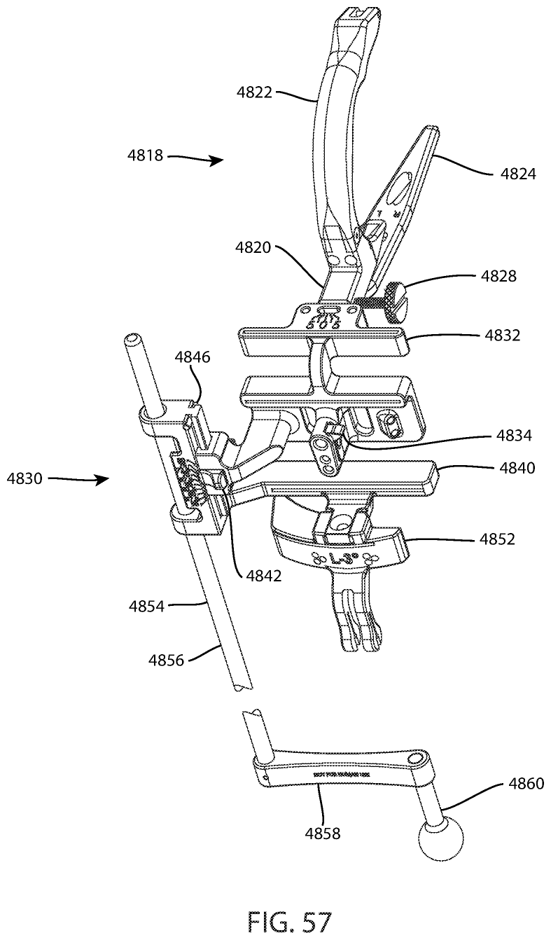

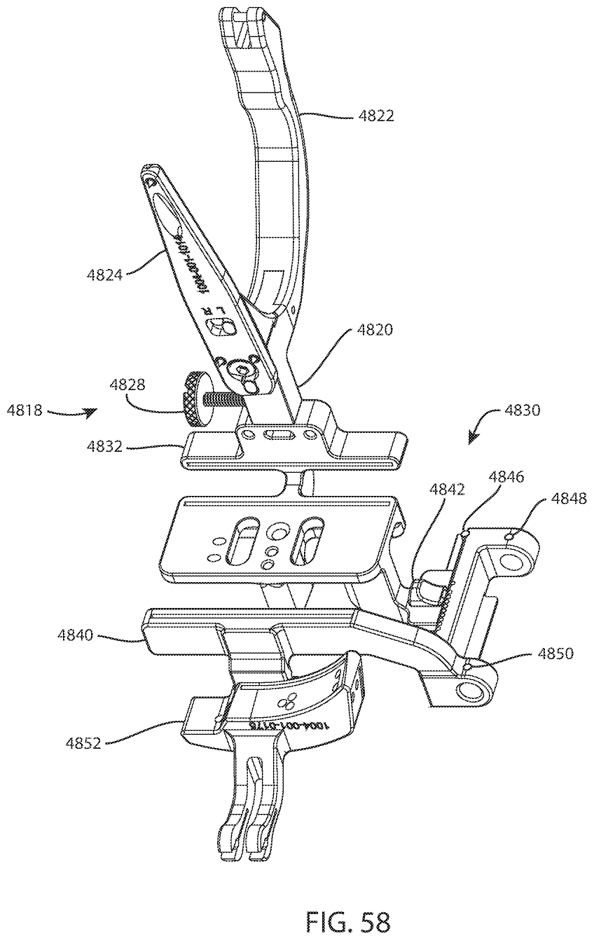

FIGS. 57-60 show the femoral alignment assembly 4818 and the cut guide assembly 4830 from different directions, operatively assembled and exploded to show component parts. The femoral extension rod 4826 and the tibial alignment rod assembly 4862 are not shown in FIGS. 57-60. The tibial alignment rod assembly 4854 is not shown in FIGS. 58-60.

The foot holder assembly 4802 may be the foot holder assembly 870, 1870, or 2870 of U.S. Pat. No. 10,568,650, or the foot holder assembly 3950 or 4100 of U.S. patent application Ser. No. 16/287,976, or foot holder 4500. The foot holder assembly 4802 is shown including an Achilles tendon alignment guide 4868 which may be the Achilles tendon alignment guide 4108 of U.S. patent application Ser. No. 16/287,976.

The femoral target 4816 may be mounted on a femoral support arm assembly (not shown), such as femoral support arm assemblies 786, 1786, or 2786 of U.S. Pat. No. 10,568,650, or the femoral support arm assembly 4410 of U.S. patent application Ser. No. 16/287,976, or the support apparatus 4504. The femoral target 4816 may be positioned over the femoral head center 120.

The femoral alignment assembly 4818 may be related to the base 10 and alignment rod 156, base 302 and femoral extension rod 306, base 502 and femoral extension rod 506, base 1502 and femoral extension rod assembly 1506, base 2502 and femoral extension rod assembly 2506 of U.S. Pat. No. 10,568,650; base 3502 and femoral extension rod assembly 3524, base 4402 and femoral extension rod 4406 of U.S. patent application Ser. No. 16/287,976; or base 4514 and femoral extension rod 4550. In this paragraph, reference to a base and corresponding rod includes all intervening structure between the base and rod, as well as component parts of the base and rod. The femoral alignment assembly 4818 may include a body 4820, a handle 4822, a base 4824, a femoral extension rod 4826, a thumbscrew 4828, a screw 4864, and a pin 4866. The body 4820, handle 4822, and base 4824 may be fixed together, and may optionally be fabricated as a single unitary part. The femoral extension rod 4826 may be considered a separate apparatus from the femoral alignment assembly 4818. A distal end of the femoral extension rod 4826 is hinged to a proximal end of the handle 4822, and a proximal end of the femoral extension rod is shown coupled to the femoral target 4816.

The cut guide assembly 4830 may be related to the cut guide assembly 30; cut guide assembly 1321, tibial extension rod assembly 1511, and/or cut guide 1240; or cut guide assembly 3010, tibial extension rod assembly 2511, and/or cut guide 2964 of U.S. Pat. No. 10,568,650; cut guide assembly 3840; or pin guide assembly 4300 and corresponding cut guides 4412, 4414, 4416 of U.S. patent application Ser. No. 16/287,976; or pin guide 4552 and corresponding cut guides 4620, 4638. These references include intervening structure and subcomponent parts. The cut guide assembly 4830 may include a first femoral body 4832 or femoral apparatus, a pin lock button 4834, a pin lock spring 4836, a pin lock screw 4838, a second femoral body 4840, a size lock button 4842, a size lock spring 4844, a tibial retaining pin 4846, first and second rod retaining pins 4848, 4850, a tibial cut guide 4852, and an offset tibial alignment rod assembly 4854. The second femoral body 4840 and the tibial cut guide 4852 together may be referred to as a tibial apparatus. The tibial alignment rod assembly 4854 may be considered a separate apparatus from the cut guide assembly 4830. The tibial alignment rod assembly 4854 may include a rod 4856, an offset bar 4858, and a spherical target-engaging part 4860.

A telescoping tibial alignment rod assembly 4862 is shown in FIG. 56 superimposed with the tibial alignment rod assembly 4854 to indicate that either one may be used interchangeably in the cut guide assembly 4830.

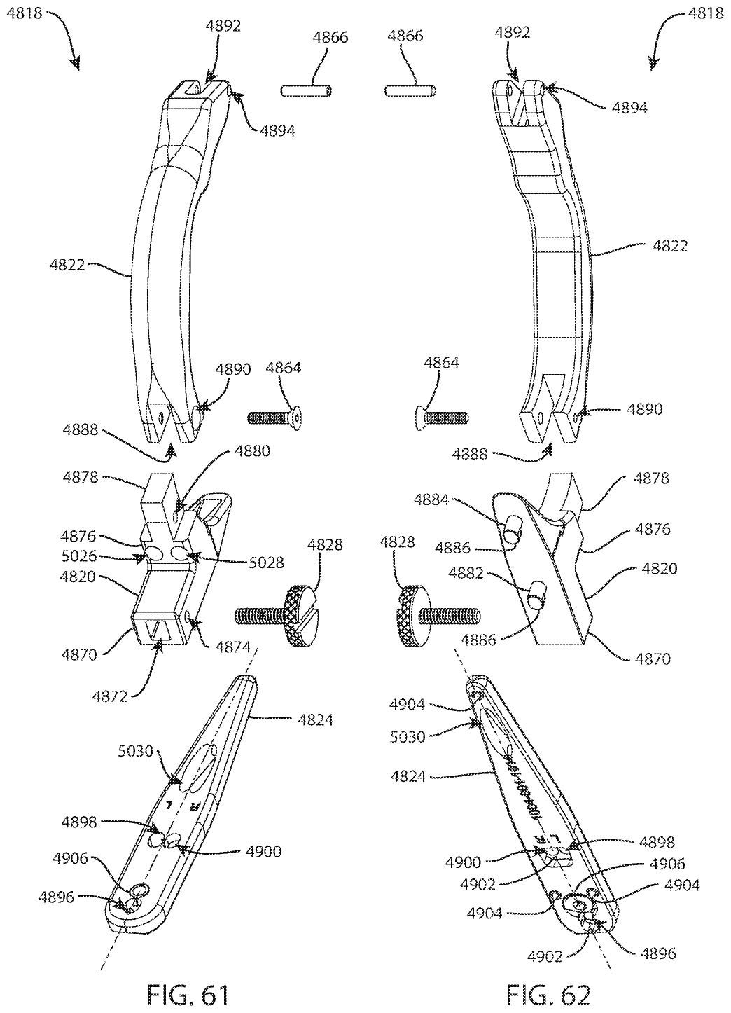

Referring to FIGS. 61-62, the body 4820 of the femoral alignment assembly 4818 includes a distal boss 4870 which surrounds a socket 4872. A rectangular socket 4872 is shown. An internally threaded hole 4874 extends through at least one side wall of the boss 4870 to intersect the socket 4872. The hole 4874 receives the thumbscrew 4828. A boss 4876 extends anteriorly and terminates in a tab 4878. A rectangular boss 4876 and tab 4878 are shown. A hole 4880 extends transversely through the tab 4878. Posts 4882, 4884 extend posteriorly from a posterior bone-facing side of the body 4820. Each post 4882, 4884 terminates in a unilaterally enlarged foot 4886. Oblique holes 5026, 5028 extend through the boss 4876.

The handle 4822 includes a distal slot 4888 which receives the tab 4878 of the body 4820. A hole 4890 extends transversely through the handle 4822 and across the slot 4888. When the tab 4878 is in the slot 4888, the holes 4880, 4890 are coaxial. The holes 4880, 4890 receive the screw 4864. At least a portion of hole 4880 or hole 4890 is internally threaded to match the screw 4864. The handle 4822 includes a proximal slot 4892 which receives the distal end of the femoral extension rod 4826 (see FIG. 56). A hole 4894 extends transversely through the handle 4822 and across the slot 4892. A comparable hole extends transversely through the distal end of the femoral extension rod 4826. When the distal end of the femoral extension rod 4826 is in the slot 4892, the holes are coaxial. The holes receive the pin 4866 to form a hinge joint between the handle 4822 and the femoral extension rod 4826.

The base 4824 includes three oblong holes 4896, 4898, 4900. Each hole includes a unilateral shelf 4902. The hole 4896 receives the post 4882 of the body 4820. The hole 4898 or 4900 receives the post 4884 for a right or left configuration as labeled. The feet 4886 engage the shelves 4902. The base 4824 may include one or more frictional elements, such as spikes 4904 protruding from the posterior bone-facing surface. The base 4824 may include a cam 4906 like cam 512 associated with base 502 of U.S. Pat. No. 10,568,650. The cam 4906 locks and unlocks the base 4824 and body 4820. An oblique hole 5030 extends through the base 4824 along an anterior-inferior to posterior-superior trajectory. The hole 5030 may be non-circular, and may be thought of as the projection, superposition, or intersection of holes 5026, 5028 of the body 4820 when the body and base are coupled together in the right or left configuration. The holes 5026, 5028, 5030 may enable visualization of the central anterior distal femoral cortex, optionally by use of a scope through the holes.

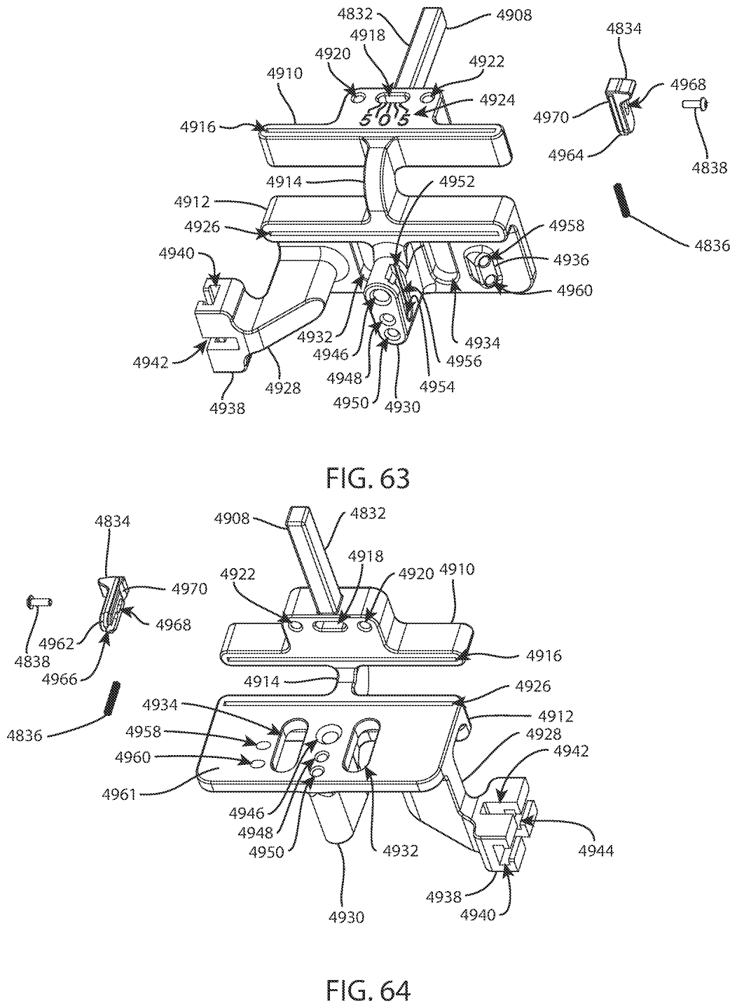

Referring to FIGS. 63-64, the first femoral body 4832 of the cut guide assembly 4830 includes a proximal boss 4908 or arm which is received in the socket 4872 of the body 4820 of the femoral alignment assembly 4818 and secured by tightening the thumbscrew 4828. The first femoral body 4832 includes a distal cut guide portion 4910 and an anterior cut guide portion 4912 coupled together by an elbow 4914.

The distal cut guide portion 4910 includes a saw slot 4916, an oval hole 4918, holes 4920, 4922, and indicia 4924. The saw slot 4916 is for making a distal femoral resection 206. The saw slot 4916 and the oval hole 4918 are elongated along a medial-lateral direction and centered medial-lateral relative to the boss 4908. The oval hole 4918 is proximal to the saw slot 4916, with reference to the femur 100 (FIG. 56). The holes 4920, 4922 are medial and lateral to the oval hole 4918, respectively. The holes 4920, 4922 receive bone pins to secure the first femoral body 4832 to the femur 100. The holes 4920, 4922 extend parallel to each other along an anterior-posterior direction relative to the femur 100. The saw slot 4916, oval hole 4918, and holes 4920, 4922 extend through the distal cut guide portion 4910 along an anterior-posterior direction relative to the femur 100.

The anterior cut guide portion 4912 includes a saw slot 4926, elbow 4928 or stalk, boss 4930, oval holes 4932, 4934, and boss 4936. The saw slot 4926 is for making an anterior femoral resection 214. The saw slot 4926 is elongated along a medial-lateral direction and centered medial-lateral relative to the boss 4908 and elbow 4914. The elbow 4928 originates posterior to a medial end of the saw slot 4926 and extends distally and medially to terminate in a free end 4938 with an undercut channel 4940, a slot 4942, and a blind hole 4944 (not visible in FIGS. 63-64). The undercut channel 4940 extends along an anterior-posterior direction relative to the femur 100. The slot 4942 extends along a proximal-distal direction and cuts across the undercut channel 4940. The hole 4944 is in the bottom of the slot 4942 where the slot crosses the undercut channel 4940.

The boss 4930 originates posterior to the saw slot 4926 and extends distally. The boss 4930 has an oval cross-sectional shape that is elongated along an anterior-posterior direction relative to the femur 100. The boss 4930 is centered medial-lateral relative to the boss 4908 and elbow 4914. The oval holes 4932, 4934 are medial and lateral to the boss 4930, respectively. The oval holes 4932, 4934 are elongated along an anterior-posterior direction relative to the femur 100. The oval holes 4918, 4932, 4934 may enable visualization of the femur 100, may reduce component weight, and/or may improve part cooling after autoclaving (if made of an autoclavable material). The boss 4930 includes three parallel holes 4946, 4948, 4950 located from anterior to posterior in the boss and extending along a proximal-distal direction. The holes 4946, 4948, 4950 receive bone pins to secure the first femoral body 4832 to the femur 100. An undercut channel 4952 extends part way along a lateral side of the boss 4930. A longitudinal hole 4954 extends into the blind end of the undercut channel 4952. A transverse blind hole 4956 extends into the bottom of the undercut channel 4952 between the holes 4946, 4948. The saw slot 4926, the oval holes 4932, 4934, and the holes 4946, 4948, 4950 extend through the anterior cut guide portion 4912 along a proximal-distal direction relative to the femur 100. The boss 4936 originates posterior to a lateral end of the saw slot 4926 and extends obliquely along an antero-lateral direction. The boss 4936 includes two holes 4958, 4960 located from anterior to posterior in the boss and extending through the anterior cut guide portion 4912 along the same oblique antero-lateral direction as the boss 4936. The holes 4958, 4960 receive bone pins to secure the first femoral body 4832 to the femur 100. The proximal side 4961 of the anterior cut guide portion 4912 directly abuts a distal surface of a medial distal condyle of the femur 100 in use, thus it may be referred to as a femur-contacting feature.

The pin lock button 4834 of the cut guide assembly 4830 includes a pair of longitudinal ribs 4962, 4964, which together with the overall shape of the pin lock button 4834 are complementary to the undercut channel 4952 of the anterior cut guide portion 4912 so that the pin lock button 4834 is received in the undercut channel 4952. A longitudinal groove 4966 extends part way along a medial side of the pin lock button 4834. An oval hole 4968 extends through the pin lock button 4834 and is elongated parallel to the groove 4966 and ribs 4962, 4964. A pin-contacting area 4970 is provided on the medial side of the pin lock button 4834 to press against a bone pin in hole 4946 to retain the first femoral body 4832 to the bone pin. The pin lock spring 4836 of the cut guide assembly 4830 is received in the hole 4954 of the first femoral body 4832 and the groove 4966 of the pin lock button 4834 and biases the pin lock button toward an anterior position. The pin lock screw 4838 of the cut guide assembly 4830 extends through the oval hole 4968 and threads into the hole 4956 to retain the pin lock button 4834 to the first femoral body 4832 and to limit the anterior-posterior travel of the pin lock button.

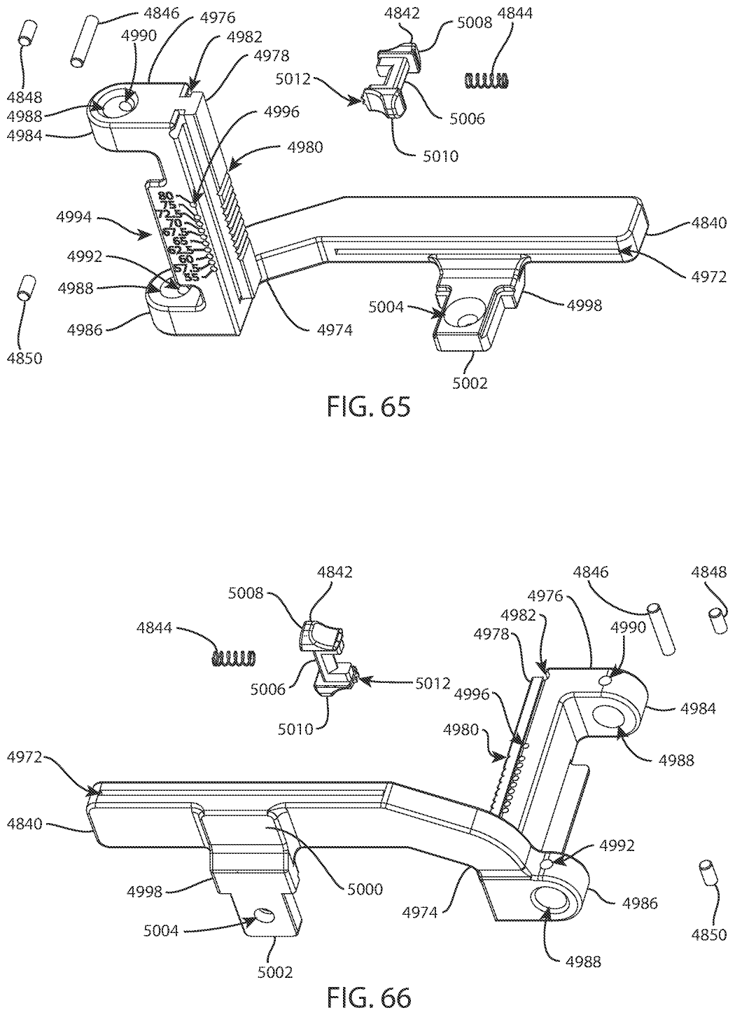

Referring to FIGS. 65-66, the second femoral body 4840 of the cut guide assembly 4830 includes a saw slot 4972 for making a posterior femoral resection 220. The saw slot 4972 is elongated along a medial-lateral direction and extends through the second femoral body 4840 along a superior-inferior direction relative to the femur 100. An elbow 4974 or stalk originates from a medial end of the saw slot 4972 and extends medially and anteriorly before terminating in a free end 4976. An undercut rail 4978 extends along an anterior-posterior direction relative to the femur 100 along a lateral side of the elbow 4974. The undercut rail 4978 is complementary to the undercut channel 4940 of the first femoral body 4832. Several grooves 4980 extend transversely across a middle portion of a lateral side of the undercut rail 4978 along a proximal-distal direction. A hole 4982 extends along a proximal-distal direction through the undercut rail 4978 at the free end 4976. Proximal and distal bosses 4984, 4986 extend medially from the proximal and distal ends of the elbow 4974, respectively. A hole 4988 extends along an anterior-posterior direction relative to the femur 100 through the bosses 4984, 4986. Holes 4990, 4992 extend through side walls of the bosses 4984, 4986 to intersect the hole 4988. Indicia 4994 may be included along a distal side of the elbow 4974. The indicia 4994 may be associated with a pattern of holes 4996 which may optionally receive indexing pins (not shown). The second femoral body 4840 includes a boss 4998 that originates posterior to a central portion of the saw slot 4972 and extends distally. A tibia-contacting feature 5000 may be a visually distinct platform on the posterior side of the second femoral body 4840 at the origin of the boss 4998, as shown. Preferably, the tibia-contacting feature 5000 is adapted to contact a central anterior portion of the tibial plateau in the vicinity of the base of the tibial eminence, or a provisional tibial resection in this area. As the boss 4998 extends distally, it narrows to an undercut rail 5002. A hole 5004 extends through the undercut rail 5002.

The size lock button 4842 includes a bridge 5006 extending between opposite end bodies 5008, 5010. A medial side of the bridge 5006 has a V-shape for engaging one of the grooves 4980 of the second femoral body 4840. A medial side of at least one of the end bodies 5008, 5010 includes a notch 5012 of about the same size as the holes 4996 of the second femoral body 4840. The size lock spring 4844 is received in the hole 4944 of the first femoral body 4832 and the bridge 5006 of the size lock button 4842 is received in the slot 4942 so that the size lock spring 4844 presses against the bridge 5006. The undercut rail 4978 of the second femoral body 4840 is received in the undercut channel 4940 of the first femoral body 4832 so that the end bodies 5008, 5010 of the size lock button 4842 are on proximal and distal sides of the undercut rail 4978, respectively, with the notch 5012 on the distal side with the indicia 4994. The tibial retaining pin 4846 is received in the hole 4982 to retain the second femoral body 4840 to the first femoral body 4832. As the second femoral body 4840 slides relative to the first femoral body 4832, the V-shape of the bridge 5006 engages each groove 4980 in turn and the notch 5012 aligns with each hole 4996 or indicia 4994 to indicate a corresponding femoral implant size matching the anterior-posterior distance between the saw slots 4926, 4972. If indexing pins are present in holes 4996, they may positively engage the notch 5012 to supplement the resistance provided by the V-shape of the bridge 5006 engaging the groove 4980.

Referring to FIGS. 56-57 and 65-66, the tibial alignment rod assembly 4854 may be the tibial alignment rod 4308 of U.S. patent application Ser. No. 16/287,976. The rod 4856 is received in the hole 4988. The pin 4848 is received in the hole 4990 and a longitudinal groove of the rod 4856. The pin 4850 is received in the hole 4992 and the longitudinal groove of the rod 4856. The pins 4848, 4850 in the groove retain the tibial alignment rod assembly 4854 to the second femoral body 4840 and prevent the tibial alignment rod assembly from rotating about a central longitudinal axis of the rod 4856 so that the tibial alignment rod assembly is only free to slide along an anterior-posterior direction relative to the femur 100.

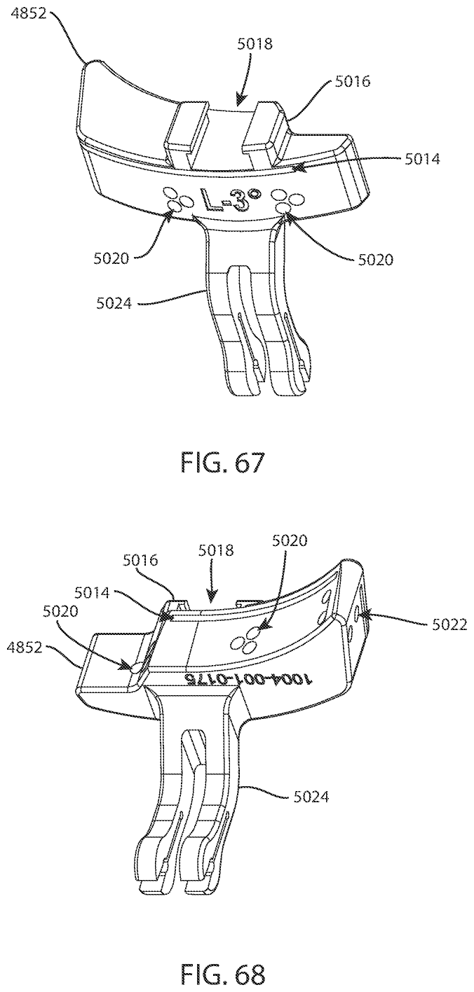

Referring to FIGS. 67-68, the tibial cut guide 4852 of the cut guide assembly 4830 includes a saw slot 5014 for making a proximal tibial resection 210. The saw slot 5014 is elongated along a medial-lateral direction and extends through the tibial cut guide 4852 along a proximal-distal direction relative to the femur 100. A boss 5016 extends anteriorly relative to the femur 100 and includes an undercut channel 5018 that extends along a proximal-distal direction. The undercut channel 5018 receives the undercut rail 5002 of the second femoral body 4840. A thumbscrew (not shown) may thread into the hole 5004 and press against the bottom of the undercut channel 5018 to lock the tibial cut guide 4852 to the second femoral body 4840. A pattern of holes 5020 may extend through the tibial cut guide 4852 along a proximal-distal direction. A pair of holes 5022 may extend through the tibial cut guide 4852 along an oblique medial-posterior direction. The holes 5020, 5022 may receive bone pins to secure the tibial cut guide to the tibia 104. The tibial cut guide 4852 is shown with a bifurcated distal arm 5024 for optional snap fit connection to the tibial alignment rod assembly 4862.

Referring to FIGS. 56 and 69-72, a surgical method for resecting a knee joint for knee arthroplasty may include one or more of the following steps in any order:

Referring to FIG. 56, providing the foot holder assembly 4802, wherein a distal tibial target of the foot holder assembly 4802 is mounted on a bridge in a location anterior to a medial-lateral center of an ankle joint, optionally anterior to the Achilles tendon alignment guide 4868. Placing a patient's foot and lower leg in the foot holder assembly 4802, optionally so that the patient's Achilles tendon is received in the Achilles tendon alignment guide and the distal tibial target is directly anterior to the patient's Achilles tendon. Fixing the patient's foot and lower leg in the foot holder assembly 4802 and fixing the foot holder assembly to an operating table.

Providing the femoral support arm assembly 4410 or 4504. Fixing the support arm assembly to an operating table with a cross bar of the femoral support arm assembly 4804 over the patient's hips and a femoral head center target mounted to the cross bar and aligned over the center of the patient's femoral head.

Creating an incision over the patient's knee joint.

Inserting a base 4824 of a femoral alignment assembly 4818 between a central distal anterior cortical surface of the femur 100 and the overlying soft tissues so that the base is against the central distal anterior cortical surface of the femur 100 and generally aligned with the femoral shaft axis.

While the base is so positioned against the femur 100, placing a proximal end of the femoral extension rod 4826 in the femoral head center target 4816 so that the central longitudinal axis of the femoral extension rod lies along the mechanical axis 202 of the femur 100 in an anterior view of the femur 100. In other words, the femoral extension rod 4826 passes over the center 120 of the femoral head and the medial-lateral center of the distal femoral epicondyles.

Providing a cut guide assembly 4830, operatively assembled. Coupling the cut guide assembly 4830 to the femoral alignment assembly 4818. This step may be done before or after the femoral alignment assembly 4818 is positioned against the femur 100 and along the mechanical axis 202 of the femur 100. When the cut guide assembly 4830 is coupled to the femoral alignment assembly 4818, the first femoral body 4832 preferably abuts the distal aspect of at least one distal femoral condyle.

While the femoral alignment assembly 4818 is positioned against the distal anterior femur 100 and along the femoral mechanical axis 202, and while the cut guide assembly 4830 is coupled to the femoral alignment assembly, placing the spherical target-engaging part 4860 of the tibial alignment rod assembly 4854 in the distal tibial target of the foot holder assembly 4802.

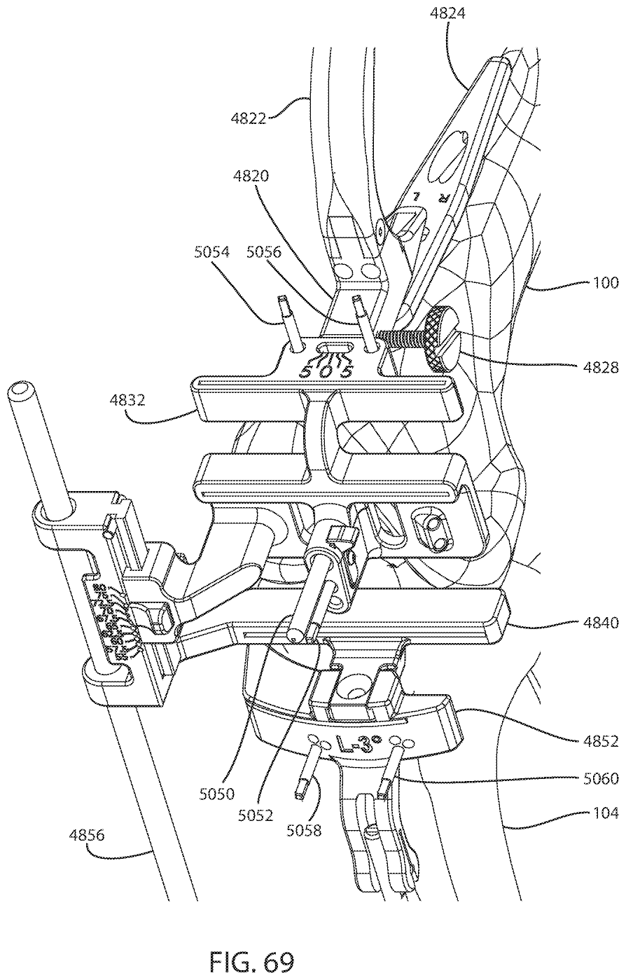

Referring to FIG. 69, while the femoral alignment assembly 4818 is positioned against the distal anterior femur 100 and along the femoral mechanical axis 202, the cut guide assembly 4830 is coupled to the femoral alignment assembly, and the spherical target-engaging part 4860 is in the distal tibial target, inserting a bone pin 5050 through hole 4946 of the first femoral body 4832 of the cut guide assembly 4830. Alternatively, the preceding step and this step may involve inserting the bone pin 5050 through hole 4946 and rotating the coupled-together femoral alignment assembly 4818 and cut guide assembly 4830 about the bone pin 5050 in hole 4946 until the spherical target-engaging part 4860 of the tibial alignment rod assembly 4854 can be placed in the distal tibial target.

After the bone pin 5050 has been inserted through hole 4946 into the distal femur 100 and while the spherical target-engaging part 4860 of the tibial alignment rod assembly 4854 is in the distal tibial target, there may be an optional sequence of steps including inserting a bone pin through hole 4918 in line with the “0°” indicia 4924, removing the tibial alignment rod assembly 4854 from the distal tibial target, and rotating the cut guide assembly 4830 about the bone pin 5050 in hole 4946, as limited by the bone pin in hole 4918, to further adjust external rotation.

Inserting bone pins through holes 4920, 4922, 4948, 4950, 4958, and/or 4960 to secure the first femoral body 4832 to the femur 100. Bone pins 5052, 5054, 5056 are shown in holes 4948, 4920, and 4922, respectively. This step may occur after the bone pin 5050 has been inserted through hole 4946, or at the same time. Preferably, pins 5050, 5052 are parallel to each other, parallel to the femoral mechanical axis in an anterior view of the femur 100, parallel to (or in a defined orientation relative to) the bone-facing side of the base 4824 in a lateral view of the femur 100, and positioned along Whiteside's Line on the distal end of the femur 100; pins 5054, 5056 are parallel to each other, perpendicular to pins 5050, 5052, and positioned along a line that is perpendicular to the femoral mechanical axis in an anterior view of the femur 100; and pins 5058, 5060 are parallel to each other and to pins 5050, 5052 (or at a defined slope), and positioned along a line that is perpendicular to the tibial mechanical axis in an anterior view of the tibia 104.

While the first femoral body 4832 is secured to the femur 100, moving the second femoral body 4840 anteriorly or posteriorly relative to the femur 100 and the first femoral body 4832 until the tibia-contacting feature 5000 contacts the tibial plateau. Moving the second femoral body 4840 anteriorly or posteriorly to the closest femoral implant size setting, as per the size lock button 4842 versus the indicia 4994, grooves 4980, and/or holes 4996.

Sliding the tibial cut guide 4852 proximally or distally relative to the femur 100, the first femoral body 4832, and the second femoral body 4840 until the tibial cut guide 4852 abuts the proximal anterior tibia. While the second femoral body 4840 and the tibial cut guide 4852 are so positioned, inserting bone pins through one or more holes 5020 and/or 5022 to secure the tibial cut guide 4852 to the proximal tibia. Bone pins 5058, 5060 are shown in holes 5020.

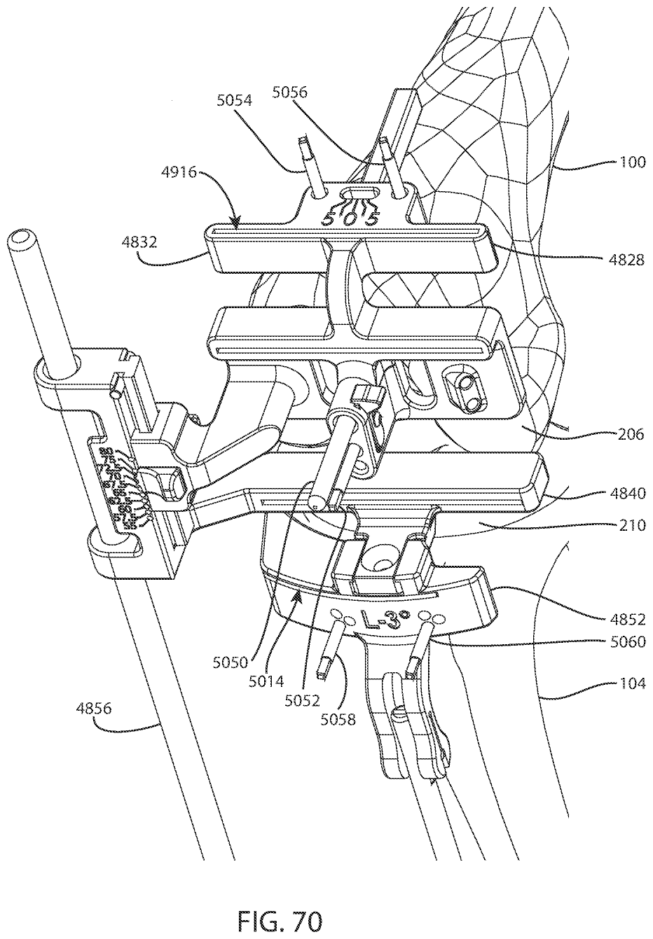

Referring to FIG. 70, after the bone pin 5050 has been inserted through hole 4946 into the distal femur 100, and optionally before any other bone pins are inserted through the cut guide assembly 4830, there may be an optional step of disconnecting the femoral alignment assembly 4818 from the cut guide assembly 4830 and removing the femoral alignment assembly 4818 from the knee. This step may involve temporarily removing the cut guide assembly 4830 from the bone pin 5050 before disconnecting and removing the femoral alignment assembly 4818, then replacing the cut guide assembly 4830 on the bone pin 5050.

Making a proximal tibial resection 210 by actuating a saw through the saw slot 5014. Making a distal femoral resection 206 through the saw slot 4916.

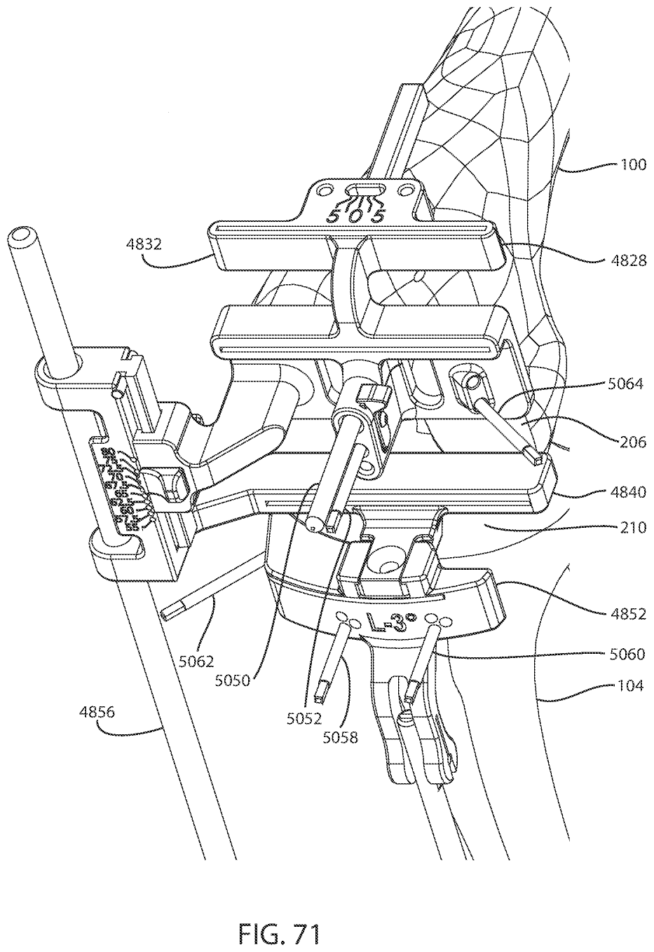

Referring to FIG. 71, optionally advancing the cut guide assembly 4830 proximally relative to the femur 100 so that the first femoral body 4832 contacts the distal femoral resection 206 and the tibial cut guide 4852 contacts the proximal anterior tibia 104. This step may include removing bone pins 5054, 5056 from holes 4920, 4922 and sliding the first femoral body 4832 along bone pins 5050, 5052 in holes 4946, 4948. Inserting bone pins through holes 4958, 4960, and/or 5022. Bone pin 5062 is shown in hole 5022 and bone pin 5064 is shown in hole 4960. The oblique orientation of these bone pins and holes prevents the cut guide assembly 4830 from sliding distally.

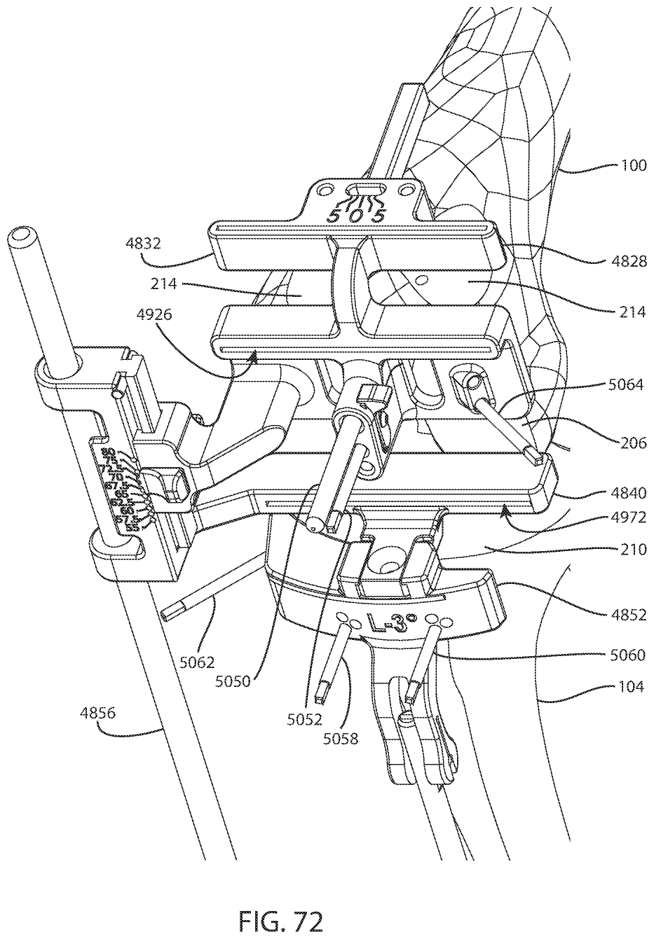

Referring to FIG. 72, making an anterior femoral resection 214 through the saw slot 4926. Making a posterior femoral resection 220 through the saw slot 4972. At least the proximal tibial resection 210, anterior femoral resection 214, and posterior femoral resection 220 are made perpendicular to Whiteside's Line due to the orientation established by pins 5050, 5052.

Optionally, bone pins 5054, 5056 may be removed from holes 4920, 4922 while the anterior and posterior femoral resections 214, 220 are being made, and bone pins 5050, 5052, 5064 may be removed from holes 4946, 4948, 4950, 4958, and/or 4960 while the distal femoral resection 206 is being made. Therefore, preferably the sequence of bone pin insertion into and removal from holes 4920, 4922, 4946, 4948, 4950, 4958, and/or 4960 is coordinated with the sequence of making the resections 206, 214, 220, 210, which may be made in any order.

The illustrated apparatus provides saw slots 4916, 4926, 4972, 5014 for at least the distal femoral resection 206, anterior femoral resection 214, posterior femoral resection 220, and proximal tibial resection 210, respectively. The anterior and posterior chamfer cuts 216, 218 may be made by removing the cut guide assembly 4830, leaving bone pins 5050, 5052 in the distal aspect of the femur 100, placing a separate chamfer cut guide (not shown) on the bone pins, and making the chamfer cuts 216, 218. The chamfer cut guide may be a generally triangular part with one side for contacting the distal femoral resection 206, with the saw guided along the remaining two planar exterior surfaces versus operating within saw slots. Optionally, one of skill in the art will appreciate that the illustrated cut guide assembly 4830 may be modified to include saw slots or planar guide surfaces for the chamfer cuts 216, 218, wherein each chamfer saw slot or guide surface is movable relative to the anterior and/or posterior saw slots 4926, 4972 in accordance with the design rationale for the femoral implant size range.

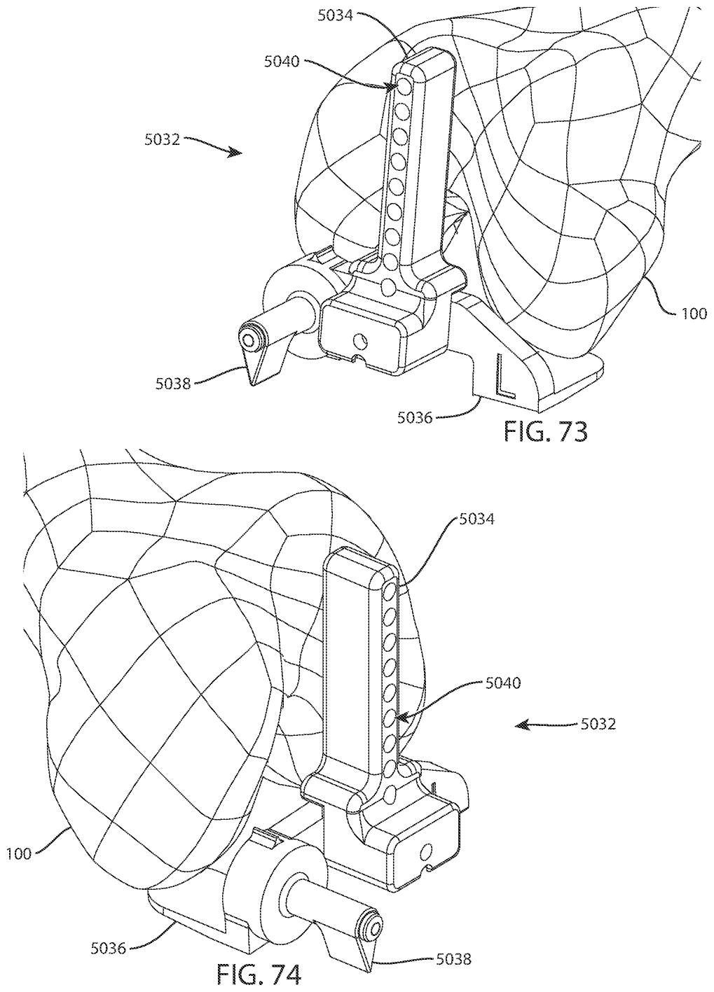

Referring to FIGS. 73-74, a Whiteside's angle gage 5032 is shown. This instrument includes a generally vertical post 5034 that is used to measure the angle of Whiteside's Line of a knee before a distal femoral resection 206 has been made. The post 5034 may snap onto a baseplate 5036, for example a baseplate of a posterior referencing implant sizer/drill guide. The baseplate 5036 may include an actuator that can be used to control the orientation of the post 5034 relative to the baseplate 5036. The actuator may take the form of a knob 5038 or dial. Turning the knob 5038 clockwise or counterclockwise may change the angle of the post 5034 relative to the baseplate 5036. When the post 5034 is visually aligned with Whiteside's Line, the corresponding angle may be indicated on a scale adjacent to the knob 5038. The post 5034 may include a linear pattern of holes 5040, through which one or more k-wires or bone pins may be inserted to directly reference (contact) the femoral intercondylar notch to identify Whiteside's Line.

In alternative embodiments, various elements of the Whiteside's angle gauge 5032 may be omitted and/or replaced with other features. For example, the baseplate 5036 need not contact the posterior condyles of the femur, but may instead contact one or more other aspects of the femur. The actuator need not be a knob 5038; rather, any device that causes the post 5034 to pivot relative to the baseplate 5038 may be used. The post 5034 need not be straight and/or elongated, but rather, may simply have one straight edge that can be visually aligned with Whiteside's Line. The holes 5040 are optional. Further, recording the orientation of the post 5034 on the scale is optional; indeed, the orientation may be recorded in various different ways, including manually by the surgeon.

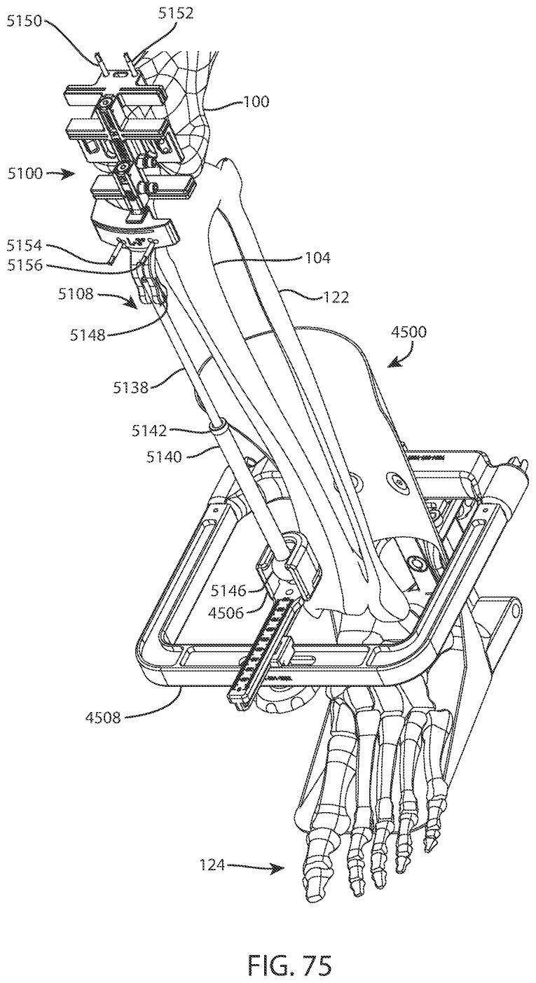

FIGS. 75-86 illustrate selected steps in a method of making femoral and tibial resections for knee arthroplasty, and related apparatus. These steps may occur after the step shown in FIGS. 17-20, and may occur instead of the steps shown in FIGS. 69-72.

Referring to FIG. 17, after inserting the bone pins 4538, 4540, removing the femoral pin guide 4512, femoral extension rod 4550, and bone pins 4538, 4540, leaving holes 4539, 4541 (FIG. 32) in the distal end of the femur 100.

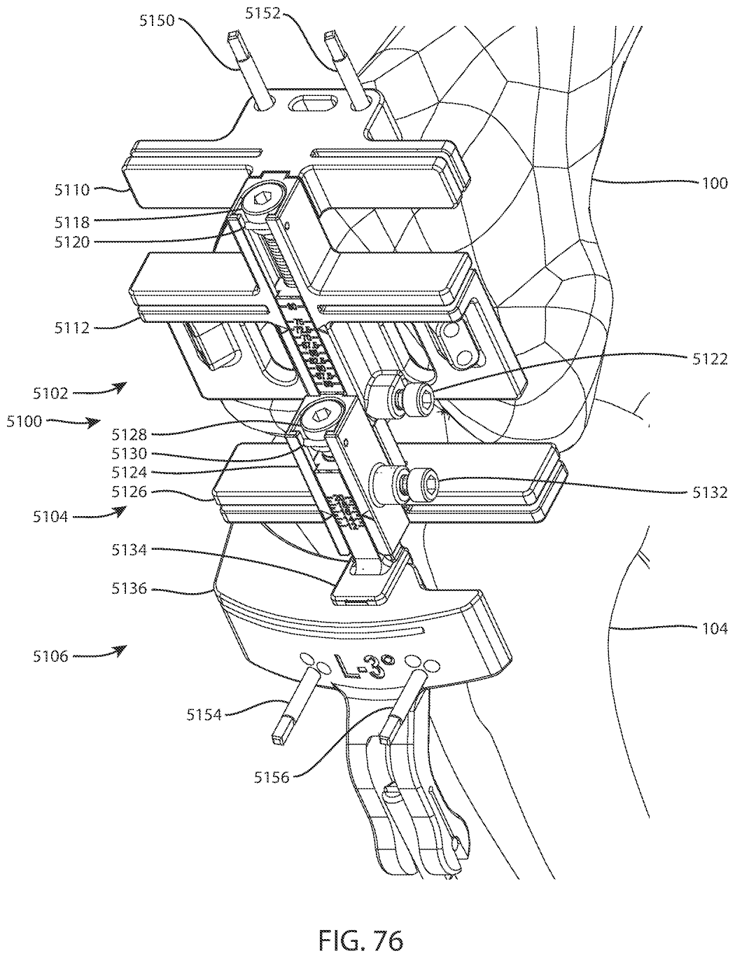

Referring to FIGS. 75-76, coupling a cut guide assembly 5100 to the femur 100, and coupling a distal target-engaging part 5146 of a tibial alignment rod assembly 5108 to the ankle center target 4506, which is in turn coupled to the bridge 4508 of the foot holder assembly 4500, which is in turn coupled to an operating table (not shown). The cut guide assembly 5100 may be similar to the cut guide assembly 4830 of FIGS. 56-60 and 63-68. One structural difference is that the interconnections between the first femoral body 4832, second femoral body 4840, and tibial alignment rod assembly 4854 of the cut guide assembly 4830 are located medial to the saw slots 4916, 4926, 4972, 5014; whereas the analogous interconnections of the cut guide assembly 5100 are centrally located relative to the saw slots. Other similarities and differences will be discussed below. The cut guide assembly 5100 may include the tibial alignment rod assembly 5108, or the tibial alignment rod assembly may be considered a separate apparatus from the cut guide assembly. The tibial alignment rod assembly 5108 may include an inner rod 5138, an outer rod 5140, a sleeve 5142, a ring 5144, a target-engaging part 5146, and a pin 5148. The tibial alignment rod assembly 5108 may be related to the extension rods or alignment rods 156, 306, 313, 506, 511, 1506, 1511, 2506, 2511 of U.S. Pat. No. 10,568,650; or extension rods or alignment rods 3524, 4308, 4406 of U.S. patent application Ser. No. 16/287,976; or extension rods or alignment rods 4550, 4558, 4826, 4854, 4862. Preferably, the tibial alignment rod assembly 5108 may be the tibial extension rod assembly 1511 or 2511 of U.S. Pat. No. 10,568,650, or the tibial alignment rod assembly 4862.

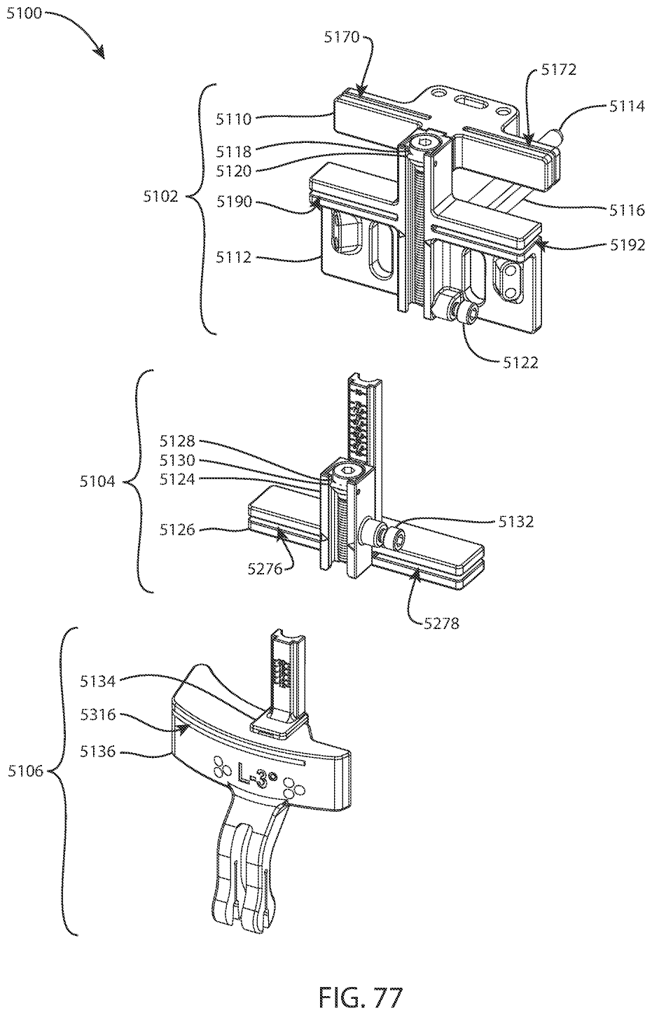

Referring to FIG. 77, the cut guide assembly 5100 is shown exploded into three part groups or sub-assemblies: a first femoral group 5102 or femoral apparatus, a second femoral group 5104, and a tibial group 5106. The second femoral group 5104 and the tibial group 5106 together may be referred to as a tibial apparatus.

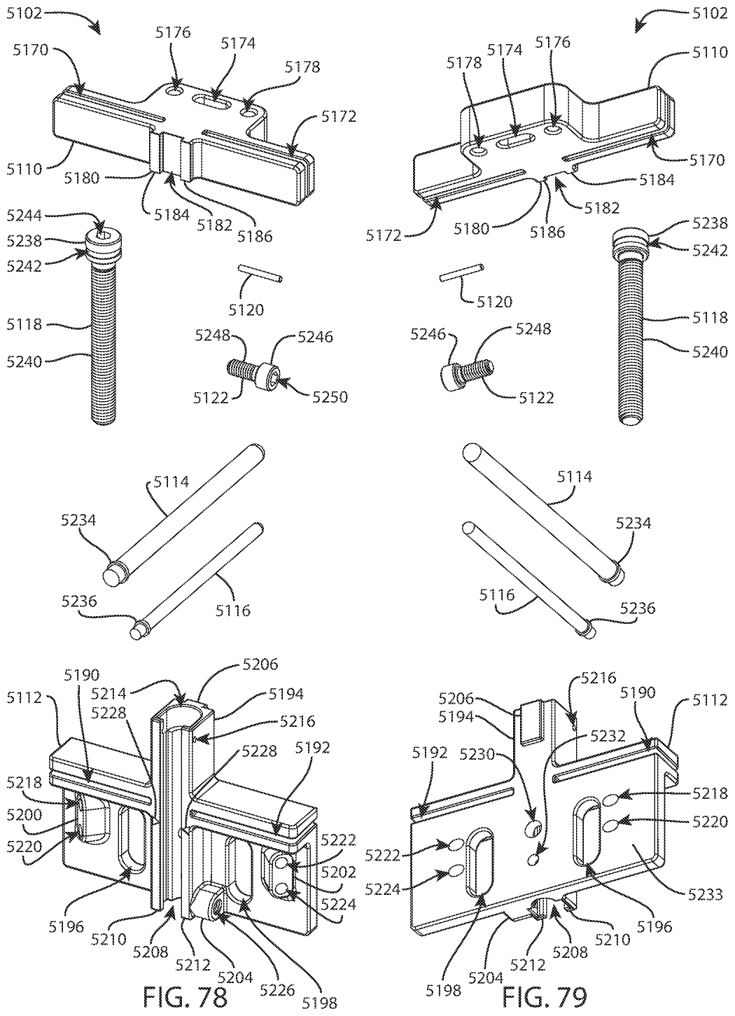

Referring to FIGS. 78-79, the first femoral group 5102 may include a first femoral body 5110, a second femoral body 5112, a first post 5114, a second post 5116, a bolt 5118, a bolt pin 5120, and a screw 5122. The first femoral group 5102 is analogous to the first femoral body 4832 of the cut guide assembly 4830.

The first femoral body 5110 is analogous to the distal cut guide portion 4910. The first femoral body 5110 includes saw slots 5170, 5172, an oval hole 5174, holes 5176, 5178, and an interconnection feature 5180. The saw slots 5170, 5172 are for making a distal femoral resection 206 while avoiding sawing the first and second posts 5114, 5116. The saw slots 5170, 5172 and the oval hole 5174 are elongated along a medial-lateral direction. The oval hole 5174 is proximal to the saw slots 5170, 5172, in use, relative to the femur 100. The holes 5176, 5178 are medial and lateral to the oval hole 5174. The holes 5176, 5178 receive bone pins to secure the first femoral body 5110 to the femur 100. The holes 5176, 5178 are parallel. The saw slots 5170, 5172, oval hole 5174, and holes 4920, 4922 extend through the first femoral body 5110 along an anterior-posterior direction relative to the femur 100. The interconnection feature 5180 is shown as a short rectangular boss that is centrally located on a distal side of the first femoral body 5110. A groove 5182 extends across the boss along an anterior-posterior direction relative to the femur 100, resulting in a pair of walls 5184, 5186 along either side of the groove 5182.

Referring to FIGS. 63-64 and 78-79, in an embodiment, the first femoral body 5110 may be modified to connect to the body 4820, base 4824, and handle 4822 in a manner similar to the first femoral body 4832, as seen best in FIGS. 57-58.

The second femoral body 5112 is analogous to the anterior cut guide portion 4912 of the first femoral body 4832 of the cut guide assembly 4830. The second femoral body 5112 includes saw slots 5190, 5192, a boss 5194, oval holes 5196, 5198, bosses 5200, 5202, a boss 5204, o and an interconnection feature 5206. The saw slots 5190, 5192 are for making an anterior femoral resection 214. The saw slots 5190, 5192 are elongated along a medial-lateral direction and located on either side of the boss 5194. The boss 5194 extends anteriorly from an anterior side of the second femoral body 5112, and has a substantially rectangular exterior shape. An undercut channel 5208 extends along a distal side of the boss 5194, resulting in a pair of walls 5210, 5212 along the medial and lateral sides of the channel 5208. The channel 5208 and walls 5210, 5212 continue across the entire anterior-posterior dimension of the second femoral body 5112. The anterior end of the channel 5208 includes an enlarged semicircular alcove 5214. A hole 5216 extends through the walls 5210, 5212 and across the channel 5208 at the level of the alcove 5214. The hole 5216 receives the bolt pin 5120. The oval holes 5196, 5198 are medial and lateral to the boss 5194, respectively. The oval holes 5196, 5198 are elongated along an anterior-posterior direction relative to the femur 100. The oval holes 5196, 5198 may enable visualization of the femur 100, may reduce component weight, and/or may improve part cooling after autoclaving (if made of an autoclavable material). The saw slots 5190, 5192 and the oval holes 5196, 5198 extend through the second femoral body 5112 along a proximal-distal direction relative to the femur 100. The bosses 5200, 5202 are analogous to the boss 4936, and are located medial and lateral to the oval holes 5196, 5198. The boss 5200 originates posterior to a medial end of the saw slot 5190 and extends obliquely along an antero-medial direction. The boss 5200 includes two holes 5218, 5220 located from anterior to posterior in the boss and extending through the second femoral body 5112 along the same oblique antero-medial direction as the boss 5200. The boss 5202 originates posterior to a lateral end of the saw slot 5192 and extends obliquely along an antero-lateral direction. The boss 5202 includes two holes 5222, 5224 located from anterior to posterior in the boss and extending through the second femoral body 5112 along the same oblique antero-lateral direction as the boss 5202. The holes 5218, 5220, 5222, 5224 receive bone pins to secure the second femoral body 5112 to the femur 100. The boss 5204 extends laterally from the lateral posterior portion of the wall 5212. The boss 5204 includes an internally threaded hole 5226 which extends through the boss and wall 5212 to communicate with the channel 5208. The hole 5226 receives the screw 5122. Indicia 5228 may be present along the distal side of at least one of the walls 5210, 5212, such as the pair of arrowheads shown. The interconnection feature 5206 is shown as a short rectangular boss that is located on a proximal or superior side of the boss 5194. Holes 5230, 5232 extend through the second femoral body 5112 in parallel along a proximal-distal direction through the bottom of the channel 5208. The holes 5230, 5232 receive the first and second posts 5114, 5116, respectively. The proximal side 5233 of the second femoral body 5112 directly abuts a distal surface of a medial distal condyle of the femur 100 in use, thus it may be referred to as a femur-contacting feature.

The first post 5114 is analogous to the bone pin 4538 or 5050. The second post 5116 is analogous to the bone pin 4540 or 5052. Each may be a substantially cylindrical part, with the first post 5114 optionally having a larger outer diameter as shown. The posts 5114, 5116 may include circumferential flanges 5234, 5236, respectively, near one end. The opposite ends may be blunt.

The bolt 5118 may include a head 5238 and an externally threaded shaft 5240 extending from the head. The head 5238 may include a circumferential exterior groove 5242 and a torque input feature 5244, such as the hex socket shown. The groove 5242 receives the bolt pin 5120. The torque input feature 5244 connects to a driver, hex key, or the like to turn the bolt 5118.

The screw 5122 may include a head 5246 and an externally threaded shaft 5248 extending from the head. The head 5246 may include a torque input feature 5250, such as the hex socket shown. The torque input feature 5250 connects to a driver, hex key, or the like to turn the screw 5122.

The first femoral group 5102 may be assembled by performing some or all of the following steps in any order: orienting the first femoral body 5110 and second femoral body 5112 as shown in FIG. 77 and inserting the interconnection feature 5206 in the groove 5182; optionally fixing the interconnection feature 5206 in the groove 5182; inserting the first post 5114 in the hole 5230 so that the flange 5234 abuts the proximal side of the second femoral body 5112; inserting the second post 5116 in the hole 5232 so that the flange 5236 abuts the proximal side of the second femoral body 5112; optionally fixing the first post 5114 in the hole 5230 and the second post 5116 in the hole 5232; inserting the bolt 5118 in the channel 5208 so that the head 5238 is received in the alcove 5214 and the shaft 5240 extends within the channel 5208; inserting the bolt pin 5120 through the hole 5216 (both portions) and groove 5242; optionally fixing the bolt pin 5120 in at least one portion of the hole 5216; and threading the shaft 5248 of the screw 5122 into the hole 5226.

When the first femoral group 5102 is operatively assembled, the first femoral body 5110, second femoral body 5112, first post 5114, and second post 5116 may all be fixed together so that they function as a single unitary part. Optionally, these parts 5110, 5112, 5114, and 5116 may be fabricated as a single unitary part. The bolt 5118 is retained within the channel 5208 due to the bolt pin 5120 in the hole 5216 and groove 5242, and is free to rotate within the channel 5208. However, the bolt 5118 is prevented from translating along the channel 5208 by the bolt pin 5120 in the hole 5216 and groove 5242, and the head 5238 in the alcove 5214.

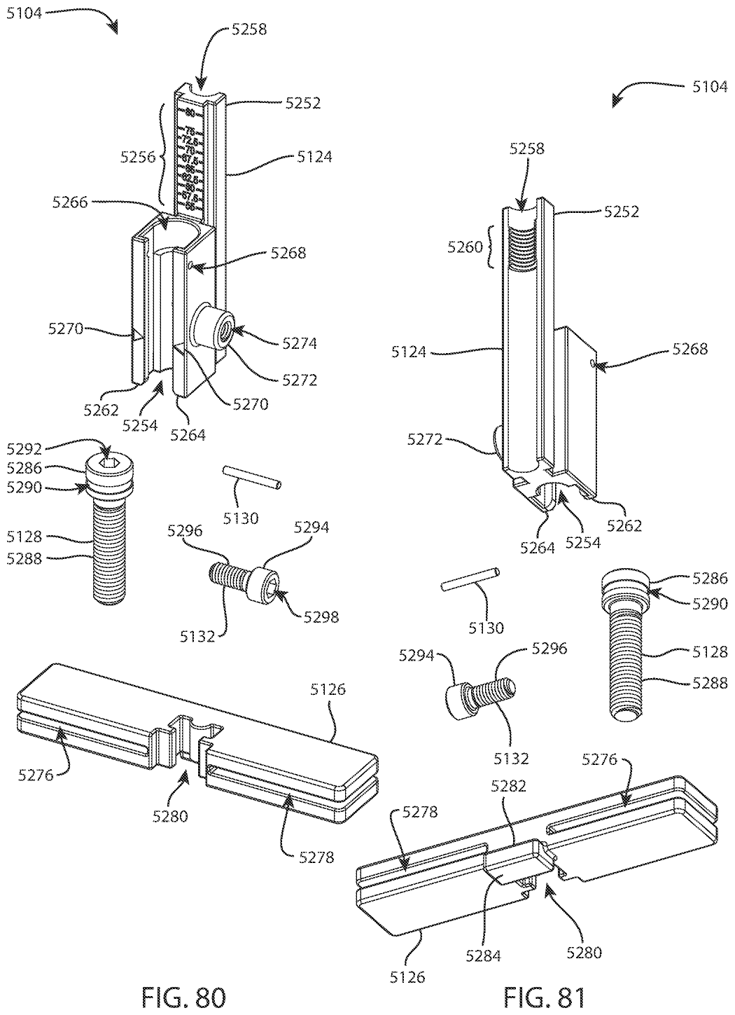

Referring to FIGS. 80-81, the second femoral group 5104 may include a third femoral body 5124, a fourth femoral body 5126, a bolt 5128, a bolt pin 5130, and a screw 5132. The second femoral group 5104 is analogous to the second femoral body 4840 of the cut guide assembly 4830.

The third femoral body 5124 is analogous to the elbow 4974 or stalk. The third femoral body 5124 includes an undercut rail 5252 for interconnection with the undercut channel 5208 of the second femoral body 5112 of the first femoral group 5102, and an undercut channel 5254 like channel 5208. The rail 5252 and the channel 5254 both extend along an anterior-posterior direction relative to the femur 100. The rail 5252 is proximal to the channel 5254. Indicia 5256 may be present on the distal side of the rail 5252, such as the numerals and lines shown. The proximal side of the rail 5252 may include a longitudinal groove 5258 of semicircular cross-section. An internally threaded portion 5260 may be included in the groove 5258 near the anterior end for engagement with the externally threaded shaft 5240 of the bolt 5118 of the first femoral group 5102; the remaining smooth portions of the groove 5258 may receive the shaft 5240 with clearance. The channel 5254 extends along the posterior distal portion of the rail 5252, and has a pair of walls 5262, 5264 along the medial and lateral sides of the channel. The anterior end of the channel 5254 includes an enlarged semicircular alcove 5266. A hole 5268 extends through the walls 5262, 5264 and across the channel 5254 at the level of the alcove 5214. The hole receives the bolt pin 5130. Indicia 5270 may be present along the distal side of at least one of the walls 5262, 5264, such as the pair of arrowheads shown. A boss 5272 extends laterally from the lateral middle portion of the wall 5264. The boss 5272 includes an internally threaded hole 5274 which extends through the boss and wall 5264 to communicate with the channel 5254. The hole 5274 receives the screw 5132.

The fourth femoral body 5126 is analogous to the portion of the second femoral body 4840 that includes the saw slot 4972. The fourth femoral body 5126 includes saw slots 5276, 5278 for making a posterior femoral resection 220. The saw slots 5276, 5278 are elongated along a medial-lateral direction and extend through the fourth femoral body 5126 along a superior-inferior direction relative to the femur 100. An undercut channel 5280 extends across a distal side of the fourth femoral body 5126 between the anterior and posterior sides. The channel 5280 is located between the saw slots 5276, 5278, and is shaped and sized to receive the posterior end of the undercut rail 5252 of the third femoral body 5124. A tibia-contacting feature 5282 extends from a superior posterior middle portion of the fourth femoral body 5126; a rectangular boss is shown. A posterior side 5284 of the tibia-contacting feature 5282 (relative to the femur 100) is adapted to contact a proximal surface of the tibia 104 in use, preferably a central anterior portion of the tibial plateau in the vicinity of the base of the tibial eminence, or a provisional tibial resection in this area.

The bolt 5128 may include a head 5286 and an externally threaded shaft 5288 extending from the head. The head 5286 may include a circumferential exterior groove 5290 and a torque input feature 5292, such as the hex socket shown. The groove 5290 receives the bolt pin 5130. The torque input feature 5292 connects to a driver, hex key, or the like to turn the bolt 5128.

The screw 5132 may include a head 5294 and an externally threaded shaft 5296 extending from the head. The head 5294 may include a torque input feature 5298, such as the hex socket shown. The torque input feature 5298 connects to a driver, hex key, or the like to turn the screw 5132.

The second femoral group 5104 may be assembled by performing some or all of the following steps in any order: orienting the third femoral body 5124 and fourth femoral body 5126 as shown in FIG. 77 and inserting the posterior end of the undercut rail 5252 into the channel 5280; optionally fixing the posterior end of the undercut rail 5252 in the channel 5280; inserting the bolt 5128 in the channel 5254 so that the head 5286 is received in the alcove 5266 and the shaft 5288 extends within the channel; inserting the bolt pin 5130 through the hole 5268 (both portions) and groove 5290; optionally fixing the bolt pin 5130 in at least one portion of the hole 5268; and threading the shaft 5296 of the screw 5132 into the hole 5274.

When the second femoral group 5104 is operatively assembled, the third and fourth femoral bodies 5124, 5126 may be fixed together so that they function as a single unitary part. Optionally, these parts 5124, 5126 may be fabricated as a single unitary part. The bolt 5128 is retained within the channel 5254 due to the bolt pin 5130 in the hole 5268 and groove 5242. The bolt 5128 is free to rotate within the channel 5208, but is prevented from translating along the channel by the bolt pin 5120 in the hole 5216 and groove 5242, and the head 5286 in the alcove 5266.

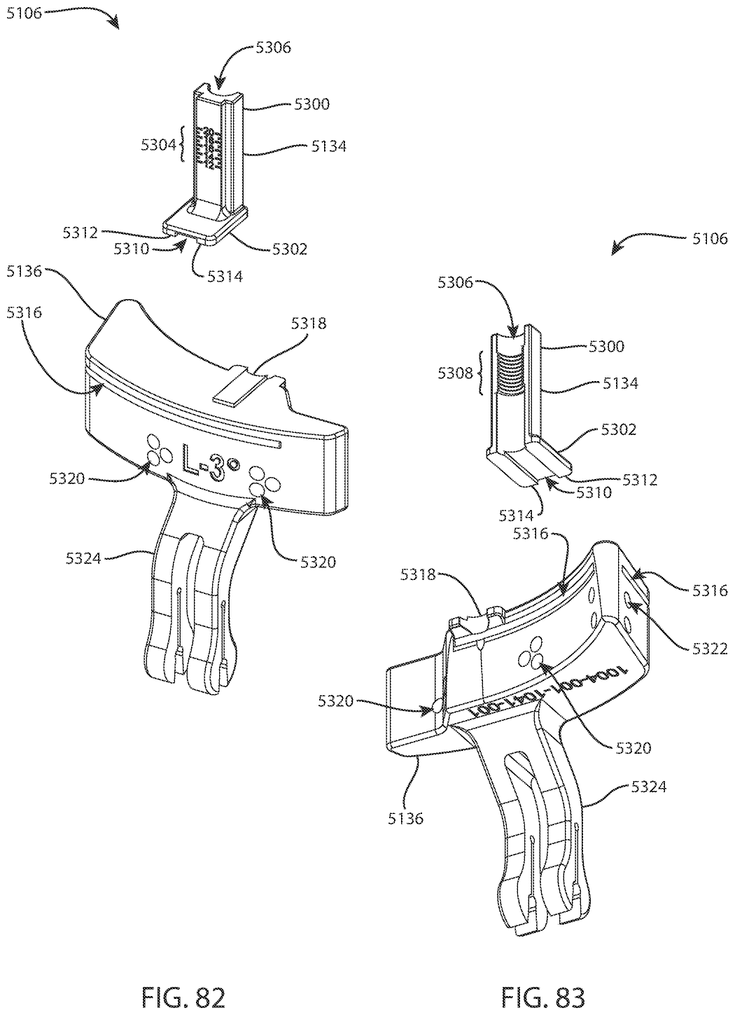

Referring to FIGS. 82-83, the tibial group 5106 may include a first tibial body 5134 and a second tibial body 5136. The tibial group 5106 is analogous to the tibial cut guide 4852 of the cut guide assembly 4830.

The first tibial body 5134 includes an undercut rail 5300 for interconnection with the undercut channel 5254 of the third femoral body 5124 of the second femoral group 5104, and an interconnection feature 5302. The rail 5300 extends along an anterior-posterior direction relative to the femur 100. Indicia 5304 may be present on the distal side of the rail 5300, such as the numerals and lines shown. The proximal side of the rail 5300 may include a longitudinal groove 5306 of semicircular cross-section. An internally threaded portion 5308 may be included in the groove 5306 near the anterior end for engagement with the externally threaded shaft 5288 of the bolt 5128 of the second femoral group 5104; the remaining smooth portions of the groove 5306 may receive the shaft 5288 with clearance. The interconnection feature 5302 is shown as a rectangular boss that extends distally from a posterior end of the rail 5300. A groove 5310 extends across the boss along a proximal-distal direction relative to the femur 100, resulting in a pair of walls 5312, 5314 along either side of the groove 5310.

The second tibial body 5136 is analogous to the portion of the tibial cut guide 4852 that includes the saw slot 5014. The second tibial body 5136 includes a saw slot 5316 for making a proximal tibial resection 210. The saw slot 5316 is elongated along a medial-lateral direction and extends through the second tibial body 5136 along a proximal-distal direction relative to the femur 100. An interconnection feature 5318 extends anteriorly relative to the femur 100; a rectangular boss is shown. A pattern of holes 5320 may extend through the second tibial body 5136 along a proximal-distal direction. A pair of holes 5322 may extend through the second tibial body 5136 along an oblique medial-posterior direction. The holes 5320, 5322 may receive bone pins to secure the second tibial body 5136 to the tibia 104. The second tibial body 5136 is shown with a bifurcated distal arm 5324 for optional snap fit connection to the tibial alignment rod assembly 5108.

The tibial group 5106 may be assembled by orienting the first tibial body 5134 and second tibial body 5136 as shown in FIG. 77; inserting the interconnection feature 5318 in the groove 5310; and optionally fixing the interconnection feature 5318 in the groove 5310.

When the tibial group 5106 is operatively assembled, the first and second tibial bodies 5134, 5136 may be fixed together so that they function as a single unitary part. Optionally, these parts 5134, 5136 may be fabricated as a single unitary part.

The cut guide assembly 5100 may be assembled by performing some or all of the following steps in any order: orienting the first and second femoral groups 5102, 5104 as shown in FIG. 77; sliding the anterior end of the rail 5252 into the posterior end of the channel 5208; rotating the bolt 5118 to engage the shaft 5240 with the internally threaded portion 5260; orienting the second femoral group 5102 and tibial group 5106 as shown in FIG. 77; sliding the anterior end of the rail 5300 into the posterior end of the channel 5254; and rotating the bolt 5128 to engage the shaft 5288 with the internally threaded portion 5308.

When the cut guide assembly 5100 is operatively assembled, rotating the bolt 5118 clockwise and counterclockwise moves the second femoral group 5104 anteriorly and posteriorly (relative to the femur 100) relative to the first femoral group 5102 to adjust the anterior-posterior distance between the saw slots 5190, 5192 and the saw slots 5276, 5278. The indicia 5228, 5256 cooperate to indicate the femoral implant component size corresponding to the anterior-posterior distance between the saw slots 5190, 5192 and the saw slots 5276, 5278. Rotating the bolt 5118 also moves the tibial group 5106 relative to the first femoral group 5102. Rotating the bolt 5128 clockwise and counterclockwise moves the tibial group 5106 anteriorly and posteriorly relative to the second femoral group 5104 to adjust the anterior-posterior distance between the saw slots 5276, 5278 and the saw slot 5316. The indicia 5270, 5304 cooperate to indicate the tibial articular insert thickness corresponding to the anterior-posterior distance between the saw slots 5276, 5278 and the saw slot 5316. Rotating the bolt 5128 also moves the tibial group 5106 relative to the first femoral group 5102.

Returning to FIG. 75, coupling the cut guide assembly 5100 to the femur 100 may include inserting the first post 5114 into the hole 4539, inserting the second post 5116 into the hole 4541, and placing the proximal side of the second femoral body 5112 in contact with a distal surface of a femoral condyle. Coupling the target-engaging part 5146 of the tibial alignment rod assembly 5108 to the ankle center target 4506 preferably includes aligning the tibial alignment rod assembly with the mechanical axis 202 of the tibia 104 in an anterior view of the tibia, which may include referencing the Achilles tendon or any of the other tibia alignment strategies discussed previously.

Actuating or adjusting the cut guide assembly 5100 to position the posterior side 5284 of the tibia-contacting feature 5282 against the proximal side of the tibia 104, such as a central anterior portion of the tibial plateau in the vicinity of the base of the tibial eminence, or a provisional tibial resection in this area. Actuating or adjusting the cut guide assembly 5100 may include turning the bolt 5118 to move the second femoral group 5104 anteriorly relative to the femur 100 until the indicia 5228, 5256 indicate the smallest femoral implant component size;

turning the bolt 5118 to move the second femoral group 5104 posteriorly relative to the femur 100 until the posterior side 5284 of the tibia-contacting feature 5282 contacts the tibial plateau, and reading the femoral implant component size indicated by the indicia 5228, 5256; optionally tightening the screw 5122 to lock the second femoral group 5104 in a fixed position relative to the first femoral group 5102; turning the bolt 5128 to move the tibial group 5106 anteriorly relative to the femur 100 until the indicia 5270, 5304 indicate the thinnest tibial articular insert; turning the bolt 5128 to move the tibial group 5106 posteriorly relative to the femur until the indicia 5270, 5304 indicate the desired tibial articular insert thickness; and/or optionally tightening the screw 5132 to lock the tibial group 5106 in a fixed position relative to the second femoral group 5104.

Inserting bone pins 5150, 5152, 5154, 5156 through holes 5176, 5178, 5320 of the cut guide assembly 5100 and into the femur 100 and tibia 104.

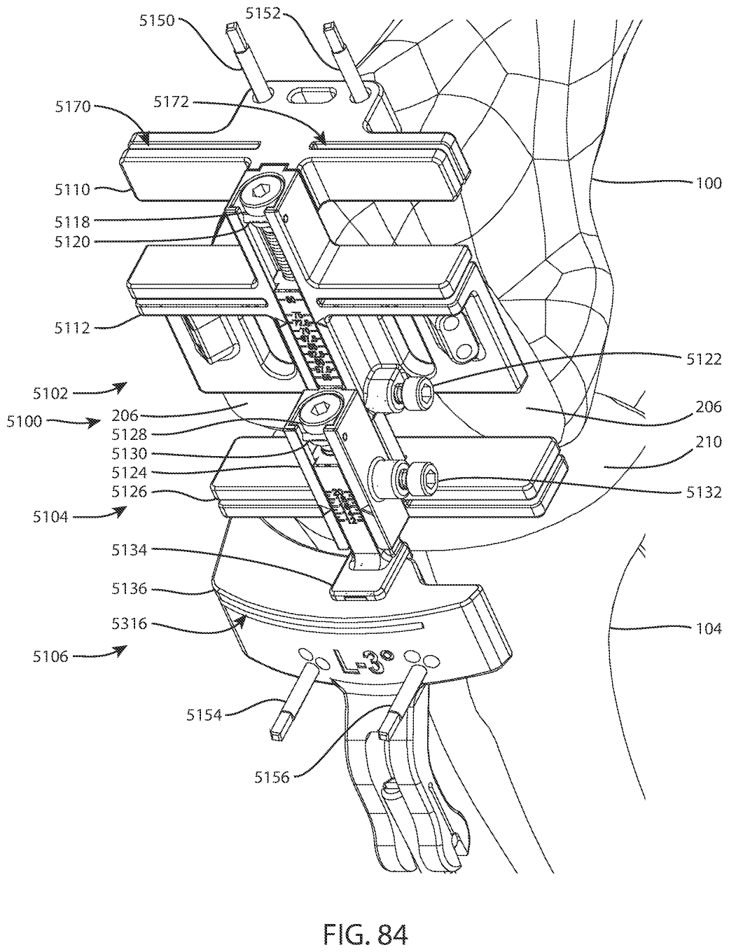

Referring to FIG. 84, actuating a saw through the saw slots 5170, 5172 to make a distal femoral resection 206; actuating a saw through the saw slot 5316 to make a proximal tibial resection 210.

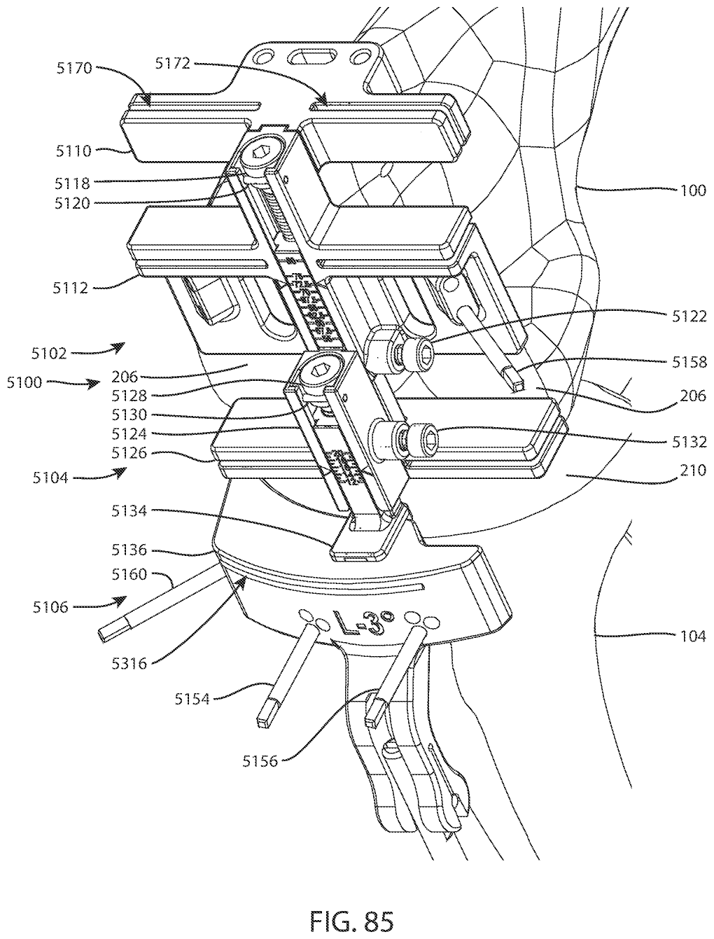

Referring to FIG. 85, removing the bone pins 5150, 5152; sliding the cut guide assembly 5100 proximally relative to the femur 100 within the holes 4539, 4541 and along the bone pins 5154, 5156 until the proximal side of the second femoral body 5112 contacts the distal femoral resection 206; inserting bone pin(s) 5158 through holes 5218, 5220, 5222, and/or 5224 into the femur 100; and inserting bone pin(s) 5160 through hole(s) 5322 and into the tibia 104.

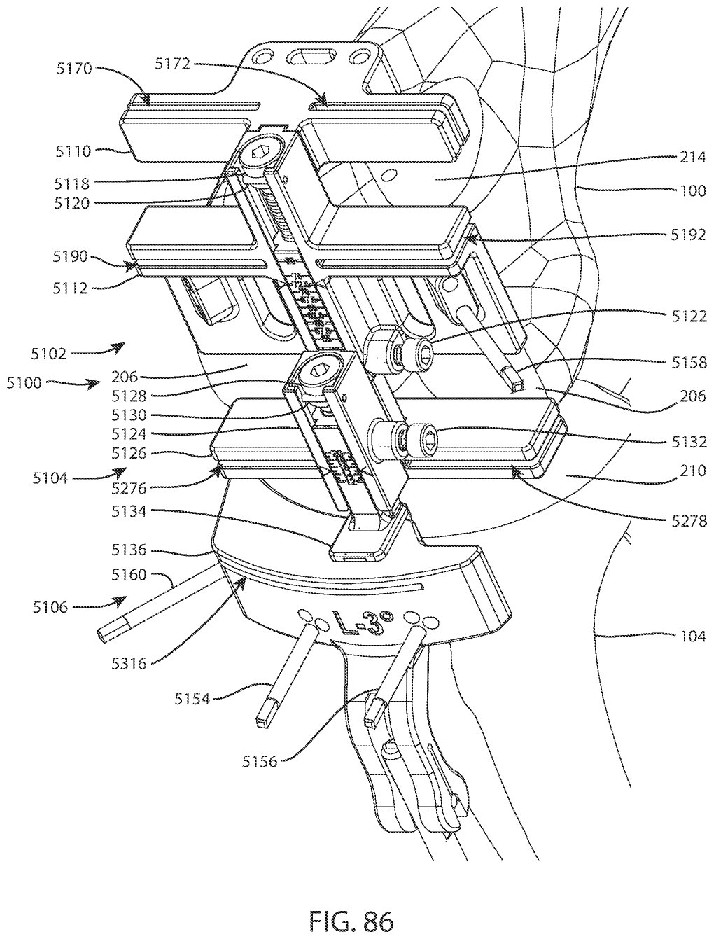

Referring to FIG. 86, actuating a saw through the saw slots 5190, 5192 to make an anterior femoral resection 214; actuating a saw through the saw slots 5276, 5278 to make a posterior femoral resection 220. At least the proximal tibial resection 210, anterior femoral resection 214, and posterior femoral resection 220 are made perpendicular to Whiteside's Line due to the orientation established by the posts 5114, 5116 in holes 4539, 4541.

The illustrated apparatus provides saw slots 5170, 5172, 5316, 5190, 5192, 5276, 5278 for at least the distal femoral resection 206, proximal tibial resection 210, anterior femoral resection 214, and posterior femoral resection 220, respectively. The anterior and posterior chamfer cuts 216, 218 may be made by removing the cut guide assembly 5100, coupling a separate chamfer cut guide (not shown) to the bone holes 4539, 4541, and making the chamfer cuts 216, 218. The chamfer cut guide may be a generally triangular part with one side for contacting the distal femoral resection 206, with the saw guided along the remaining two planar exterior surfaces versus operating within saw slots. Optionally, one of skill in the art will appreciate that the illustrated cut guide assembly 5100 may be modified to include additional bodies with saw slots or planar guide surfaces for the chamfer cuts 216, 218, wherein each chamfer body may be movable relative to the anterior and/or posterior saw slots 5190, 5192 and 5276, 5278 in accordance with the design rationale for the femoral implant size range.

The pin guide assembly 4300, 6-in-1 pin guide 4552, cut guide assembly 4830, and/or cut guide assembly 5100 may be modified to include a soft tissue tensioning apparatus (not shown) which is biased to apply tension to the collateral ligaments and/or other soft tissues of the knee as the pin or cut guide assembly is coupled to the femur 100 and tibia 104 for the purpose of balancing the soft tissues with respect to the femoral and tibial resections. The tensioning apparatus may include a spring or force actuator between the femoral multi-pin guide 4302 and tibial pin guide 4304 of the pin guide assembly 4300, between the femoral body 4554 and tibial body 4556 of the 6-in-1 pin guide 4552, between the first femoral body 4832 and second femoral body 4840 of the cut guide assembly 4830, and/or between the first femoral group 5102 and second femoral group 5104 of the cut guide assembly 5100. Multiple springs or actuators may be included, as well as multiple femur and/or tibia-contacting features. The tensioning apparatus may also include indicia or a display to show the actual or relative tension applied at one or more locations.

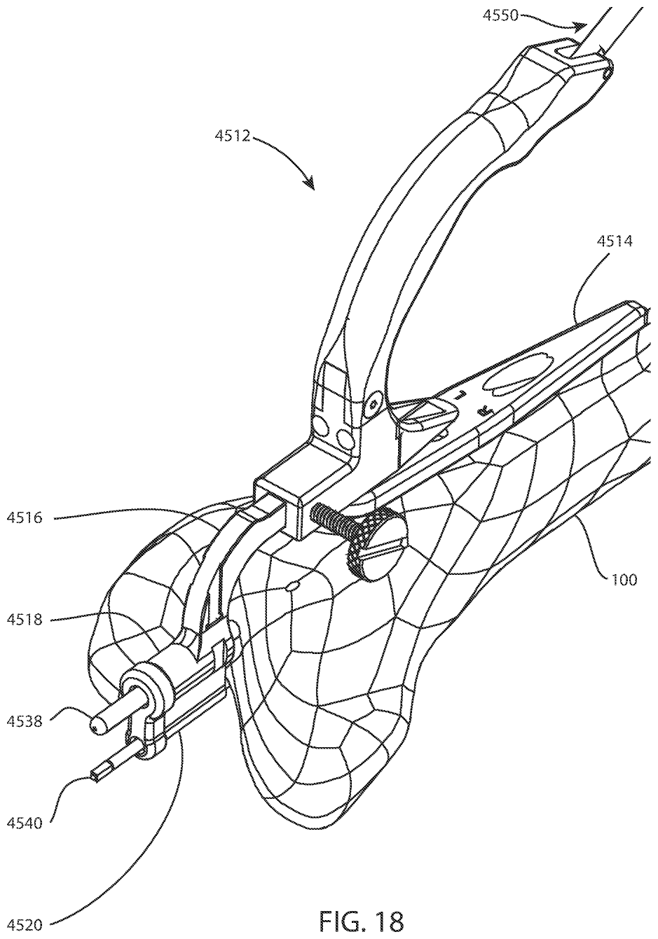

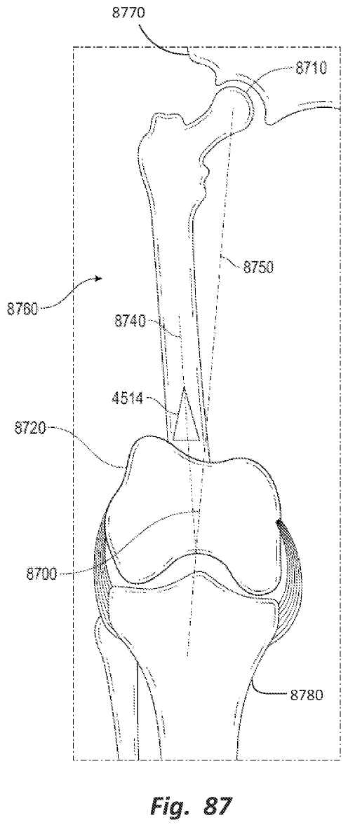

Referring to FIG. 87, in some embodiments, Whiteside's Line 8700 may be used to ascertain the position of the femoral head 8710. Many have assumed or otherwise believe that Whiteside's Line 8700 is aligned with (i.e., parallel to or coplanar with) the femoral intramedullary canal (centered on a line 8740, which may also be centered on a base, such as the base 4514 of FIGS. 17 and 18). the inventor has discovered that Whiteside's Line 8700 is, instead, aligned with (i.e., parallel to or coplanar with) the line 8750 extending between the femoral head 8710 and the distal end 8720 of the femur 8760, as shown.

Specifically, the line 8750 drawn along Whiteside's Line 8700, and bisecting the sulcus of the distal end 8720 of the femur 8760, may reside within a plane (viewed from its edge in FIG. 87) that passes from the center of the distal end 8720 of the femur 8760 to the ball of the femoral head 8710, which forms the center of rotation of the femur 8760 relative to the pelvis 8770. The line 8750 (the “kinematic line”) within this plane may extend between the centers of articulation of the femoral head 8710 and the distal end 8720 of the femur 8760. Thus, Whiteside's Line 8700 can be used as a guide for mechanical or kinematic knee replacement.

More particularly, in a traditional mechanical or kinematic approach to knee replacement, the intramedullary canal (represented by the line 8740) of the femur 8760 may be used as a reference for orientation of the femoral implant. The femoral implant may be angled at an angular offset from the intramedullary canal, at the line 8740, by a predetermined or estimated angle. A predetermined angular offset of 5° may be used in some embodiments. Although this angle may be a reasonable average angular offset, the actual angular offset between the line 8470 and the kinematic line 8750 may vary between patients (for example, between 4° and 8°). Accordingly, the 5° approximation may not provide natural motion for many patients, particularly those with curved femurs or other unusual anatomic features.

In some embodiments, the position of the femoral head 8710 may be ascertained via x-ray, and then the femoral knee implant may be oriented with reference to this location. Various rods or other instruments may be used to accomplish this, for example, via placement parallel to the kinematic line 8750. However, Whiteside's Line 8700 may provide a sufficiently accurate indication of the kinematic line 8750 without the need to take x-rays or otherwise measure the position of the femoral head 8710. This may be the case for not just humans, but for any mammal. Accordingly, this technique may also be used animal surgeries.

Referring again to the Whiteside's angle gauge 5032 of FIGS. 73-74, the Whiteside's angle gauge 5032 may be used to measure desired “external rotation” for linked instruments, or for standard unlinked instruments (that reference the posterior condyles). The angle indicated on the scale adjacent to the knob 5038 may be the arthritic compensator for vertically positioning Whiteside's Line (i.e. in line with the tibial longitudinal axis, or the line 8740 of FIG. 87).

Arthritic wear affecting the femoral condyles may determine the relative contact of the external rotation “paddles” to the two femoral condyles that cause variation from 2° 5°. This arthritic wear differential may, if not compensated for, cause error in the central axis of the femur (Whiteside's Line 8700) relative to the tibial longitudinal axis, represented by the line 8740.

Utilizing the linked alignment technique, incidence of collateral ligament imbalance may be reduced, or even eliminated. If the Orthosensor reads more than 5 units greater on the medial side, the tibia 8780 may be shaved slightly into varus.

To eliminate this axis malalignment without linked instruments, an experienced surgeon may attempt to estimate and “replace” the worn arthritic condylar cartilage. This can be a challenging procedure. The “linked” alignment guide (for example, the cut guide assembly 5100) may assist all knee surgeons in achieving anatomic femoral/tibial alignment and correct ligament balance.

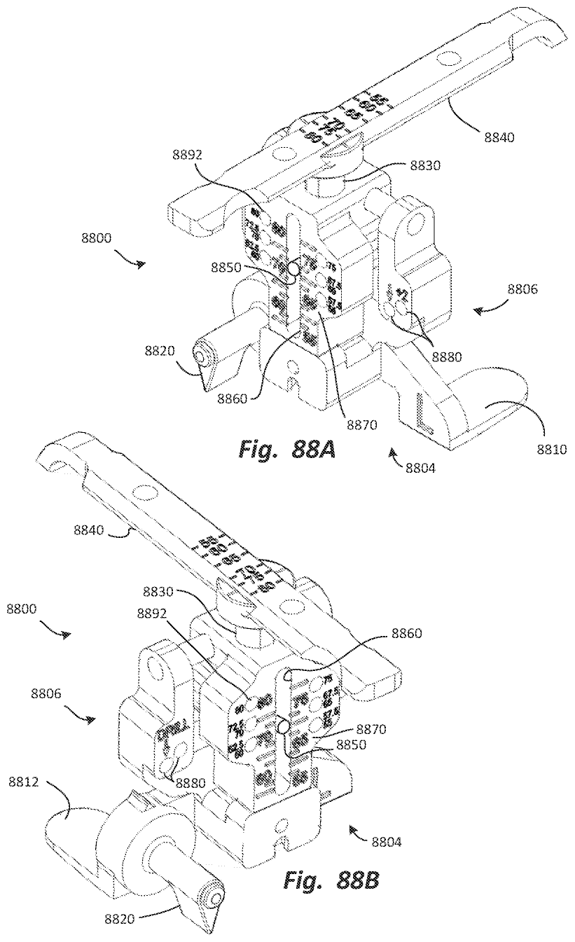

Returning to use of Whiteside's Line 8700 to obtain proper alignment of knee replacement implants, various instruments may be used to facilitate orthopedic surgeries based on this principle. As described above, an external rotation guide such as the Whiteside's angle gauge 5032 may be aligned with Whiteside's Line 8700 and used to guide resection of the femur 8760 in preparation for placement of a femoral arthroplasty implant. Such an external rotation guide may be combined with a femoral implant sizing guide so that placement and sizing may all be determined quickly and efficiently. This will be shown in connection with FIGS. 88A through 90.

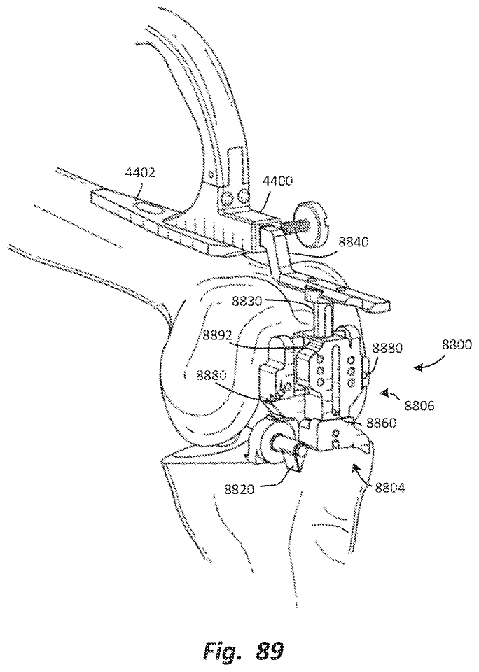

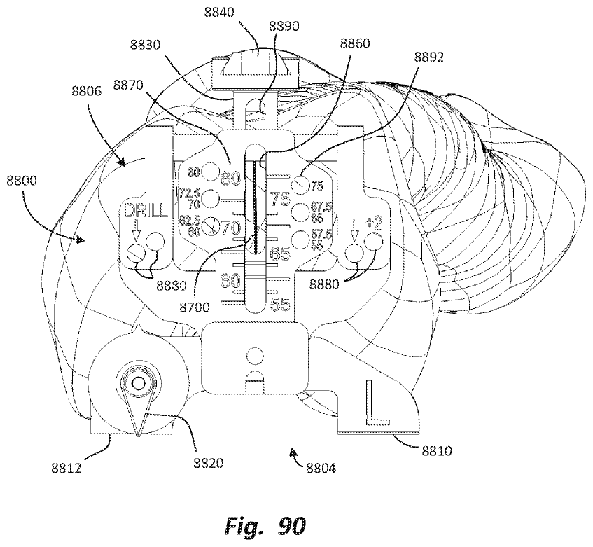

Referring to FIGS. 88A through 90, one embodiment of such a femoral implant sizing guide, or sizing guide 8800, is shown. FIGS. 88A and 88B are perspective views showing the sizing guide 8800 in isolation, FIG. 89 is a perspective view showing the sizing guide 8800 in place on the femur with anterior attachment, and FIG. 90 is an anterior view showing the sizing guide 8800 on the femur, with the anterior attachment omitted.

Separate sizing guides for left and right femurs may be used. The sizing guide 8800 may be present in place of the post 5034 of the Whiteside's angle gauge 5032 of FIGS. 73-74, and may provide the function of the post 5034. In the alternative, the Whiteside's angle gauge 5032 may be used to measure and record the orientation of Whiteside's Line as set forth above, and then the post 5034 may be removed and replaced with the sizing guide 8800.

The sizing guide 8800 may have a base 8804, which may be the same as or similar to the baseplate 5036 of FIGS. 73-74 above, with paddles 8810, 8812 that are placed on the posterior aspects of the medial and lateral femoral condyles, respectively. An angular adjustment dial 8820 at the lower left may be used to adjust the angle at which the remainder of the sizing guide 8800 extends from the paddles 8810, 8812. The angular adjustment dial may be similar to or the same as the knob 5038 of FIGS. 73-74 above.

An anterior plunger 8830 may be slidably coupled to the anterior (i.e., top in FIGS. 88A and 88B) end of the sizing guide 8800 and may receive an anterior arm 8840 that is registered on the femur, for example, by attachment to the body 4400 and the base 4402 of FIG. 12. If desired, the anterior arm 8840 may be inserted into the body 4400 in a manner similar to that of the arm 4314 of the femoral multi-pin guide 4302 described above. The anterior plunger 8830 may be secured to a pin 8850 that rides anterior-posteriorly in a slot 8860 labeled with markings 8870 indicating the anterior-posterior size of the implant that should be placed on the distal femur. The anterior plunger 8830 may pivot relative to the base 8804, along with the body 8806 of the sizing guide 8800, which may surround the anterior plunger 8830, as the angular adjustment dial 8820 is adjusted.

Whiteside's Line 8700 may be visible through the slot 8860. The anterior plunger 8830 may further have a slot 8890 that enables Whiteside's Line 8700 to be visible without obstruction. In the alternative, a window or transparent section could be formed in the anterior plunger 8830. The pin 8850, shown in FIGS. 88A and 88B, but not in FIGS. 89 and 90, may optionally be removed after the appropriate size of the femoral implant has been assessed. In the alternative, the anterior plunger 8830 may first be aligned with Whiteside's Line 8700, and then the pin 8850 may be inserted into the slot 8860 and the slot 8890 to indicate the size of the femoral implant to be used. The slot 8890 may have ridges and/or other features (not shown) that hold the pin 8850 in place at a particular location in the slot 8890.

Notably, the pin 8850 is only one of many different size indicators that may be used. For example, in alternative embodiments, markings, ridges, etchings, and/or other features on or coupled to the anterior plunger 8830 may indicate the proper size of the femoral implant. In each case, such a size indicator may be positioned a variable distance from the baseplate 5036 (for example, from the paddles 8810, 8812), so that the distance of the size indicator from the baseplate 5036 indicates the appropriate size of the femoral implant.

Since the anterior plunger 8830 may be the reference used to locate Whiteside's Line 8700, it may be referred to as a “post,” as it is performing the function of the post 5034 of the Whiteside's angle gauge 5032 of FIGS. 73-74. However, the orientation of Whiteside's Line 8700 may be measured and/or captured without the anterior plunger 8830 (for example, by visualizing Whiteside's Line 8700) through the slot 8860, with the anterior plunger 8830 removed. Thus, the body of the sizing guide 8800 may also act as a “post” by performing the function of the post 5034 of the Whiteside's angle gauge 5032.

The angular adjustment dial 8820 may be used to rotate the anterior portion of the sizing guide 8800 until the slot 8860 is parallel to Whiteside's Line 8700. If desired, a marker, such as any type of surgical marking device, may be used to darken or otherwise mark Whiteside's Line 8700 in order to make it more easily visible through the slot 8860 and the slot 8890. This may optionally be done prior to placing the sizing guide 8800 on the distal femur.

FIG. 90 shows Whiteside's Line generally centered within the slot 8860 and the slot 8890; however, in some embodiments, Whiteside's Line 8700 may be closer to one side of the slot 8860 and the slot 8890 than the other and may simply be aligned parallel with the sides of the slot 8860 and the slot 8890.

Once the sizing guide 8800 has been secured to the anterior and posterior aspects of the femur, and the slot 8860 has been aligned with Whiteside's Line 8700, pins (not shown) may be placed in the medial and lateral condyles, for example, through the two pairs of holes 8880 on the lateral and medial sides of the sizing guide 8800 (one of which has an arrow with the word “drill”). The holes 8892 closer to the slot 8860 may be used to add one or more additional (temporary) pins to temporarily restrict medial-lateral motion of the slot and/or mark the desired implant size. The proper implant size may be read from the sizing guide 8800, and the sizing guide 8800 may be removed, leaving the pins in place. Advantageously, once these pins are in place, there may be no need for further measurements, x-rays, etc. to make the needed cuts on the femur. For example, further resection steps may reference the pins placed in the femur through the sizing guide 8800.

These pins may be parallel to the kinematic line 8750 and may thus be used to guide resection of the distal femur to receive a femoral implant aligned with the kinematic line 8750. A cannulated cutter (not shown) may be placed over the pins to provide a planar distal cut on the distal end of the femur. Another cutting guide (not shown) may be coupled to the pins and used to make an anterior resection, a posterior resection, and two chamfer resections on the distal femur, resulting in five sides of an octagonal shape, angled at 135° relative to each other. The femoral implant may be secured to the resected surfaces and may provide natural articulation due to alignment of the femoral implant with the hip joint.

Various modifications may be applied to a guide as shown above. For example, in some embodiments, an anterior femoral plate may be incorporated into the guide, for example, as part of the anterior arm 8840. The plate may, for example, have 3 prongs that are placed on the anterior cortex of the center of the femur to register the sagittal plane of the femur. The anterior femoral plate may be integrated with the anterior arm or may be a separate piece from it like the body 4400 and the base 4402 described above. Other variations may be envisioned by a person of skill in the art with the aid of the present disclosure.

In some embodiments, alignment of a femoral implant based on Whiteside's Line 8700 may be used for robotic surgery. For example, a surgical robot may be programmed to, independently or with the aid of a surgeon, identify Whiteside's Line 8700 and align the femoral cuts accordingly. Advantageously, referencing Whiteside's Line 8700 in this manner may avoid the need to analyze any x-rays, CT scans, or other patient data, besides the distal femur, in order to properly locate the femoral implant on the femur.

In some embodiments, identification of Whiteside's Line 8700 may be done by the surgeon. The surgeon may use a measurement device such as the Whiteside's angle gauge 5032 of FIGS. 73-74 and/or the sizing guide 8800 of FIGS. 88A through 90 to visually assess the orientation of Whiteside's Line 8700. This orientation may be input into the robotic surgical system through any known mechanism. For example, the surgeon may read the angle from a measurement device, such as the scale, adjacent to the knob 5038 of the Whiteside's angle gauge 5032, that measures and records the angle of Whiteside's Line 8700 and enter that angle into the robotic system. In the alternative, the robotic system may be connected to the angle measurement system such that, once an element (such as the post 5034) has been aligned with Whiteside's Line, the angle is captured by the robotic system and used to orient and/or position future resections made by the robotic system, or by the surgeon with the aid of the robotic system.

In alternative embodiments, the computer system may identify Whiteside's Line and may make automatically receive its orientation. For example, any type of computer vision systems known in the art may be used. Lasers and/or other optical tools may be used to facilitate computer identification. The surgeon may mark Whiteside's Line 8700 with a surgical marker, one or more radiographic markers, and/or the like, which can facilitate detection of Whiteside's Line 8700 by the robotic system. Once the robotic system has received the orientation of Whiteside's Line 8700, the orientation may be used to orient and/or position future resections made by the robotic system, or by the surgeon with the aid of the robotic system, as mentioned above.

In some embodiments, these robotic steps may be carried out without the need to locate and/or reference the medial and/or lateral aspects of the sulci of the femur. Thus, the robotic system may not require removal and/or penetration of soft tissues adjacent to the medial and lateral collateral ligaments to properly facilitate resection of the femur. Further, in some embodiments, there may be no need to X-ray the patient to ascertain the location of the femoral head relative to the distal femur. Rather, once the distal femur is exposed, the robotic system may determine the position and/or orientation of each femoral resection based only on the position and/or orientation of Whiteside's Line 8700.

If desired, a robotic system may utilize one or more reference pins or other objects secured to the femur. For example, pins may be inserted into Whiteside's Line 8700, or into the sulci of the femur, as described above in connection with the sizing guide 8800. A robotic system may then, physically and/or optically, reference these pins in determining where resections should be made.

A wide variety of surgical techniques may use the discoveries disclosed herein for various human and animal surgeries. One exemplary technique for knee arthroplasty, referencing Whiteside's Line to provide the proper mechanical/kinematic location of the femoral implant, will be set forth below. Those of skill in the art will appreciate that many variations of this technique may be used:
1. Expose the anterior surface of the distal femur. Utilize a cobb elevator to separate the distal-anterior femoral fat pad from the anterior surface of the femur (about 10 cm in length).
2. Insert the 7 cm long, metal (or plastic) “cleated” plate (for example, the base 4402) on the anterior-distal surface of the exposed distal femur (supported by a curved handle). This plate may identify 1) The sagittal plane of the distal femur, and 2) the exact anterior surface of the femur permitting the anterior reference point needed for sizing the distal femur (anterior/posterior size of femoral prosthesis).
3. Insert the external rotation control instrument (for example, the baseplate 5036 of the Whiteside's angle gauge 5032) by sliding the two posterior femoral “paddles” (for example, paddles 8810, 8812) under the femoral condyles (knee flexed 90 degree). Connect the external rotation instrument to the extended portion of the “cleated plate” which has previously been pressed onto the anterior surface of the distal femur. Read the location of the pin 8850 (sliding inside the vertical walls of the external rotation instrument) to determine the proper size for the femoral implant.
4. Adjust coincidence of vertical slot and Whiteside's′ Line 8700 (seen visually).
5. Drill the two, 3.2 mm pin holes, in each of distal femoral condyles . . . drill hole guide provided by the Whiteside's′ Line controlled external rotation guide. These two pins (one each in the distal femoral condyles) may control 1) the sagittal plane of all cuts in the distal femur, and 2) the proper “external rotation” cuts of the distal femur, by aligning the cut planes of the distal femur at 90 degrees to the longitudinal axis of the femur. This may be possible because the longitudinal center of the sulcus of the femur (Whiteside's Line 8700) may simply be the distal extension of the longitudinal axis of the femur. Therefore, utilization of Whiteside's' Line 8700 as the longitudinal plan of the femur, may eliminate the need for any intramedullary or extramedullary femoral rod utilization for the purpose of locating the femoral head.

Notably, these method steps may be carried out by a surgeon, a surgical robot, and/or a surgeon with the aid of a surgical robot. The significant discovery of the longitudinal plan of “Whiteside's' Line” may also eliminate the need for robotic registration of hip ROM for the purpose of hip location and ultimately, determination of the longitudinal axis of the femur. The two 3.2 mm pins inserted into each of the distal femoral condyles may also permit the precision mounting of the cut guide for the distal femoral cut (instead of placing two pins in the distal-anterior cortex of the femur). Traditional TKA instrumentation may require coronal and sagittal control of the (9 mm) distal femoral cut by referencing the femoral intramedullary rod, inserted from distal to proximal. The net result may be that only two small pins can now control all cuts to the distal femur (and the proximal tibia).

Any methods disclosed herein includes one or more steps or actions for performing the described method. The method steps and/or actions may be interchanged with one another. In other words, unless a specific order of steps or actions is required for proper operation of the embodiment, the order and/or use of specific steps and/or actions may be modified.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles of the technology.

While specific embodiments and applications of the present technology have been illustrated and described, it is to be understood that the technology is not limited to the precise configuration and components disclosed herein. Various modifications, changes, and variations which will be apparent to those skilled in the art may be made in the arrangement, operation, and details of the methods and systems of the present technology disclosed herein without departing from the spirit and scope of the technology.

What is claimed is:

1. An angle gauge configured to facilitate arthroplasty of a knee joint between a femur and a tibia, the angle gauge comprising:
a base configured to contact a distal end of the femur;
a post pivotably connected to the base such that the post is visually alignable with Whiteside's Line on the femur; and
an angle capture feature configured to capture an angle of Whiteside's Line when the post is in alignment with Whiteside's Line.

2. The angle gauge of claim 1, wherein the base is configured to contact one or both posterior condylar surfaces of the femur.

3. The angle gauge of claim 1, further comprising an actuator that can be actuated to change an orientation of the post relative to the base.

4. The angle gauge of claim 1, wherein the angle capture feature comprises a scale on which the angle is indicated.

5. The angle gauge of claim 1, wherein the post comprises one or more holes that are alignable with Whiteside's Line.

6. The angle gauge of claim 5, further comprising one or more pins that can be inserted through the one or more holes in the post to directly reference Whiteside's Line to facilitate alignment of the post with Whiteside's Line.

7. The angle gauge of claim 1, wherein the post is incorporated into a sizing guide configured to indicate an appropriate size of a femoral implant to be used in arthroplasty of the knee joint.

8. The angle gauge of claim 7, wherein the sizing guide comprises a size indicator carried by the post at an adjustable displacement from the base to indicate a size of a femoral implant that is suitable for the arthroplasty.

9. The angle gauge of claim 8, wherein the post comprises an anterior plunger that is slidable, within a body, such that the size indicator is alignable with any of a plurality of markings on the body, indicating potential sizes of the femoral implant.

10. The angle gauge of claim 9, wherein at least one of the post and the body comprises a slot through which Whiteside's Line is viewable.

11. A sizing guide configured to facilitate arthroplasty of a knee joint between a femur and a tibia, the sizing guide comprising:
a base configured to contact the femur;
a post pivotably connected to the base such that the post is visually alignable parallel to Whiteside's Line on the femur; and
a size indicator carried by the post at an adjustable displacement from the base to indicate a size of a femoral implant that is suitable for the arthroplasty.

12. The sizing guide of claim 11, further comprising a body;
wherein the post comprises an anterior plunger that is slidable, within the body, such that the size indicator is alignable with any of a plurality of markings on the body, indicating potential sizes of the femoral implant.

13. The sizing guide of claim 12, wherein at least one of the post and the body comprises a slot through which Whiteside's Line is viewable.

14. The sizing guide of claim 11, wherein the base is configured to register on posterior condylar surfaces of the femur.

15. The sizing guide of claim 14, further comprising an anterior arm on an anterior end of the post, wherein the anterior arm extends from the post to register on an anterior surface of a distal end of the femur.

16. A method for carrying out knee arthroplasty, the method comprising:
exposing a distal femur;
assessing an orientation of Whiteside's Line on the distal femur; and
based on the orientation, defining a position and/or orientation of one or more resections to be made to the distal femur.

17. The method of claim 16, wherein assessing the orientation of Whiteside's Line comprises visually aligning a post of an angle gauge with Whiteside's Line.

18. The method of claim 17, wherein:
the angle gauge further comprises a base to which the post is pivotably coupled; and
visually aligning the post with Whiteside's Line comprises pivoting the post relative to the base until the post is parallel to Whiteside's Line.

19. The method of claim 17, wherein:
the angle gauge further comprises an angle capture feature; and
the method further comprises using the angle capture feature to capture the orientation.

20. The method of claim 19, further comprising:
receiving the orientation in a surgical robot; and
using the orientation to identify a position and/or orientation of one or more resections to be made to the distal femur.
